(19) United States
(12) Patent Application Publication
(10) Pub. No.: US 2023/0329916 A1
(43) Pub. Date: Oct. 19, 2023

(54) TRANSPARENT, FLEXIBLE SUBSTRATES FOR USE IN WOUND HEALING AND WEARABLE BIOELECTRONICS

(71) Applicants: Case Western Reserve University, Cleveland, OH (US); The United States Government as Represented by The Department of Veteran Affairs, Washington, DC (US)

(72) Inventors: Dhruv SESHADRI, Cleveland, OH (US); Christian ZORMAN, Cleveland, OH (US); Katherine M. BOGIE, Washington, DC (US)

(21) Appl. No.: 18/026,870

(22) PCT Filed: Sep. 21, 2021
(86) PCT No.: PCT/US2021/051265

§ 371 Date: Mar. 17, 2023

Related U.S. Application Data
(60) Provisional application No. 63/080,904, filed on Sep. 21, 2020.

(51) Int. Cl. A61F 13/02; A61L 15/24; A61F 13/00; A61N 1/04; A61L 15/44

(52) U.S. Cl. CPC A61F 13/0206; A61L 15/24; A61F 13/00063; A61N 1/04; A61L 15/44; A61F 2013/00182

(57) ABSTRACT

The present disclosure is directed to transparent, flexible substrates and, in particular, moisture-absorbing substrates for use in wound healing and wearable bioelectronics. Substrates of the present application can be formulated and used as wound dressings, electrodes, and electroceutical devices. Advantageously, in some aspects, substrates of the present application can absorb moisture (e.g., wound exudate, apocrine sweat, eccrine sweat), without swelling while also remaining transparent even with moisture absorption. In other aspects, substrates of the present application can be formulated to exhibit superior mechanical and electrical properties for application to a wide array of bioelectronic applications.

### RELATED APPLICATION

This application claims priority to U.S. Provisional Application Ser. No. 63/080,904 filed Sep. 21, 2020, entitled “TRANSPARENT, FLEXIBLE SUBSTRATES AND METHODS OF USE THEREOF”, the entirety of which is hereby incorporated by reference for all purposes.

### TECHNICAL FIELD

The present disclosure relates generally to transparent, flexible substrates and, in particular, moisture-absorbing substrates for use in wound healing and wearable bioelectronics.

### BACKGROUND

Desired properties for a wound dressing include the ability to maintain a suitable environment at the wound/dressing interface, absorb excess exudates without leakage to the surface of a dressing, provide thermal insulation, maintain mechanical and bacterial protections, allow gaseous and fluid exchanges, absorb wound odor, be nonadherent to the wound and easily removable without trauma, and be nontoxic, nonallergic, non-sensitizing, sterile and non-scaring.

As shown in Table 1 below, however, all currently available commercial wound dressings suffer from certain disadvantages.

TABLE 1
Comparative analysis of commercial wound dressing categories in the market today

| Dressings | Advantages | Disadvantages |
|---|---|---|
| Alginates | Ca²⁺—Na⁺ exchange produces gel, worn for | Requires a secondary dressing |
| | several days before change, absorb 20x | thereby increasing wound |
| | their weight | management costs |
| Foams | Comfort to patient | Non-adherent, secondary |
| | | dressing needed, produce foul |
| | | smell, |
| Gauze | Cheapest type of wound dressing, made of | Dries wound (unless impregnated |
| | cotton | w/agents) |
| Hydrocolloids | Worn for several days before dressing | Contact dermatitis, Produce foul |
| | change | smell |
| Hydrofibers | Carboxymethylcelluose fibers form gel | Less lateral wicking and less |
| | w/exudate | maceration of intact periwound |
| | | skin |
| Polymeric | Long-term wear possible (e.g. 1 week). | Efficacy dependent on exudate |
| Membrane | Useful even when no exudate present | levels |
| Dressings | | |

The use of bioelectricity to enhance wound healing is known. Epidermal electronics that conform to the skin enable non-invasive monitoring and measurement of biomechanical, physiological, and biochemical parameters relevant for human health and performance. Polydimethyl siloxane (PDMS), polyethylene terephthalate (PET), and polyimide (PI) are routinely used as substrate materials; however, their hydrophobicity limits utility in applications that involve management of body fluids, such as sweat or wound exudate.

### SUMMARY

One aspect of the present disclosure can include a wound dressing for application against a wound site of a subject. The wound dressing can comprise: a transparent, moisture absorbing layer having a wound side and an opposed outer side; and an adhesive layer that is connected to at least a portion of the wound side of the moisture absorbing layer, wherein the adhesive layer facilitates attachment of the wound dressing to a non-wounded perimeter of the wound site. The moisture absorbing layer can absorb moisture from the wound site, without swelling, to promote healing of the wound site.

Another aspect of the present disclosure can include a device comprising: a transparent, moisture absorbing layer having a wound side and an opposed outer side, wherein the moisture absorbing layer absorbs moisture, without swelling, from a wound site; an occlusive layer positioned against at least a portion of the outer side of the moisture absorbing layer, wherein the occlusive layer comprises flexible circuitry that defines a plurality of electrical contacts; and a plurality of temperature sensors coupled to the flexible circuitry. Each temperature sensor of the plurality of temperature sensors can be in electrical communication with a respective contact of the plurality of contacts of the flexible circuitry.

Another aspect of the present disclosure can include a method for monitoring healing of a wound site. The method can comprise positioning a device on a subject having a wound site. The device can comprise: a transparent, moisture absorbing layer having a wound side and an opposed outer side, wherein the moisture absorbing layer absorbs moisture, without swelling, from a wound site; an occlusive layer positioned against at least a portion of the outer side of the moisture absorbing layer, wherein the occlusive layer comprises flexible circuitry that defines a plurality of electrical contacts; and a plurality of temperature sensors coupled to the flexible circuitry. Each temperature sensor of the plurality of temperature sensors can be in electrical communication with a respective contact of the plurality of contacts of the flexible circuitry. The device can be positioned so that a first temperature sensor of the plurality of temperature sensors is positioned within or over the wound site and a second temperature sensor of the plurality of temperature sensors is positioned at a location spaced apart from the wound site. Next, a status of the wound can be determined, by a processing device, based on a temperature difference between the first temperature sensor and the second temperature sensor.

Another aspect of the present disclosure can include a method for healing a wound site of a subject. One step of the method can include applying a wound dressing or a device over the wound site. The wound dressing can comprise: a transparent, moisture absorbing layer having a wound side and an opposed outer side; and an adhesive layer that is connected to at least a portion of the wound side of the moisture absorbing layer, wherein the adhesive layer facilitates attachment of the wound dressing to a non-wounded perimeter of the wound site. The moisture absorbing layer can absorb moisture from the wound site, without swelling, to promote healing of the wound site. The device can comprise: a transparent, moisture absorbing layer having a wound side and an opposed outer side, wherein the moisture absorbing layer absorbs moisture, without swelling, from a wound site; an occlusive layer positioned against at least a portion of the outer side of the moisture absorbing layer, wherein the occlusive layer comprises flexible circuitry that defines a plurality of electrical contacts; and a plurality of temperature sensors coupled to the flexible circuitry. Each temperature sensor of the plurality of temperature sensors can be in electrical communication with a respective contact of the plurality of contacts of the flexible circuitry. A series of electrical stimulations can optionally be applied by the electrodes to the wound site. The wound dressing or the device is then left over the wound site for a period of time until the wound site is healed.

Another aspect of the present disclosure can include an electrode comprising carbon black, a thermoplastic material, and a polyol compound. The carbon black can be provided at a weight of between about 10% and 70% of a weight of the thermoplastic material and the polyol compound. The electrode can be formulated to absorb moisture without swelling.

Another aspect of the present disclosure can include a device comprising: a moisture absorbing layer having a wound side and an opposed outer side; a plurality of electrodes disposed over the wound side of the moisture absorbing layer; an occlusive layer positioned against the outer side of the moisture absorbing layer, wherein the occlusive layer comprises flexible circuitry that defines a plurality of electrical contacts; and a plurality of temperature sensors coupled to the flexible circuitry. Each electrode of the plurality of electrodes can be in electrical communication with a respective contact of the plurality of contacts of the flexible circuitry. An electrode of the plurality of electrodes can comprise carbon black, a thermoplastic material, and a polyol compound. The carbon black can be provided at a weight of between about 10% and 70% of a weight of the thermoplastic material and the polyol compound. The electrode can be formulated to absorb moisture without swelling.

Another aspect of the present disclosure can include a method for healing a wound site of a subject. One step of the method can include applying a device over the wound site. The device can comprise: a moisture absorbing layer having a wound side and an opposed outer side; a plurality of electrodes disposed over the wound side of the moisture absorbing layer; an occlusive layer positioned against the outer side of the moisture absorbing layer, wherein the occlusive layer comprises flexible circuitry that defines a plurality of electrical contacts; and a plurality of temperature sensors coupled to the flexible circuitry. Each electrode of the plurality of electrodes can be in electrical communication with a respective contact of the plurality of contacts of the flexible circuitry. An electrode of the plurality of electrodes can comprise carbon black, a thermoplastic material, and a polyol compound. The carbon black can be provided at a weight of between about 10% and 70% of a weight of the thermoplastic material and the polyol compound. The electrode can be formulated to absorb moisture without swelling. Next, a series of electrical stimulations can be applied by the electrodes to the wound site until the wound site is healed. The series of electrical stimulations can be based on a received temperature measurement and/or a received impedance measurement from between the first and second electrodes and from between each temperature sensor of the plurality of temperature sensors, respectively.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1A-D are schematic illustrations showing an exploded perspective view (FIG. 1A), an exploded side view (FIG. 1B), a top view (FIG. 10), and a bottom view (FIG. 1D) of a wound dressing constructed in accordance with the present application.

FIGS. 2A-B are schematic illustrations showing an exploded perspective view (FIG. 2A) and an assembled side view (FIG. 2B) of a wound dressing assembly constructed in accordance with another aspect of the present disclosure.

FIG. 3 is a schematic illustration showing an alternative configuration of the wound dressing assembly in FIGS. 2A-B.

FIGS. 4A-B are schematic illustrations showing a top view (FIG. 4A) and side view (FIG. 4B) of a device for monitoring healing of a wound site constructed in accordance with another aspect of the present disclosure.

FIGS. 5A-B are schematic illustrations showing an alternative configuration of the device in FIGS. 4A-B.

FIGS. 6A-B are schematic illustrations showing a perspective view (FIG. 6A) and a side view (FIG. 6B) of an electrode constructed in accordance with another aspect of the present disclosure.

FIG. 7A is a schematic illustration showing a perspective view of an alternative configuration of an electrode of the electrode in FIGS. 6A-B.

FIG. 7B is a schematic illustration showing a top view of the electrode in FIG. 7A.

FIG. 7C is a cross-sectional view taken along Line 7C-7C in FIG. 7A.

FIGS. 8A-B are schematic illustrations showing an exploded perspective view (FIG. 8A) and an assembled perspective view (FIG. 8B) of an alternative configuration of an electrode constructed in accordance with another aspect of the present disclosure.

FIGS. 9A-C are schematic illustrations showing a top view (FIG. 9A), a side view (FIG. 9B), and a bottom view (FIG. 9C) of a device (e.g., an electroceutical device) constructed in accordance with another aspect of the present disclosure.

FIGS. 10A-C are schematic illustrations showing an alternative configuration of the device in FIGS. 9A-C.

FIG. 11 is a schematic illustration showing a fabrication scheme of a wound dressing, referred to below as “AFTIDerm”, according to one aspect of the present disclosure.

FIG. 12 is a schematic illustration showing a fabrication scheme of AFTIDerm containing carbon black (CB), referred to below as “CB-AFTIDerm” or “CB-AFTIDerm electrodes”, according to another aspect of the present disclosure.

FIGS. 13(a)-(c) show fabrication of AFTIDerm and surface property characterization. FIG. 13(a) shows AFTIDerm synthesis. FIG. 13(b) is a plot showing water contact angle based on varied glycerol concentrations (wt %) (1%, 3%, 5% 7%, and 10%). FIG. 13(c) is a series of images showing AFTIDerm on the skin.

FIGS. 14(a)-(d) show mechanical testing of AFTIDerm at varied glycerol concentrations. FIG. 14(a) is a stress versus strain plot. FIG. 14(b) is a plot showing Young's Modulus versus glycerol concentration. FIG. 14(c) is a plot showing cyclic stress versus glycerol concentrations. FIG. 14(d) is a plot showing cyclic stress of AFTIDerm at 5 wt % glycerol concentration.

FIGS. 15(a)-(e) are a series of plots showing cyclic stress of AFTIDerm samples at varied glycerol concentrations (wt %): 0% glycerol (FIG. 15(a)); 1% glycerol (FIG. 15(b)); 3% glycerol (FIG. 15(c)); 7% glycerol (FIGS. 15(d)); and 10% glycerol (FIG. 15(e)).

FIG. 16 is a plot showing absorption of AFTIDerm at varied glycerol concentrations (wt %) (mean data reported; n=6 trials per glycerol concentration group).

FIG. 17 is a plot comparing absorption of AFTIDerm at 5 wt % glycerol concentration against HP Tegaderm and Absorbent Tegaderm (mean data reported; n=6 trials per glycerol concentration group).

FIGS. 18(a)-(b) show thermal transport through the AFTIDerm interface. FIG. 18(a) is a schematic detailing the experimental set-up. FIG. 18(b) is a box and whisker plot comparing the measured temperatures.

FIGS. 19(a)-(c) show translation of AFTIDerm as a wound dressing. Schematic detailing packaged wound dressing prior to ethylene oxide sterilization (FIG. 19(a)). Images of the AFTIDerm wound dressing on a chronic wound (FIG. 19(b)). Absorption of exudate by the AFTIDerm wound dressing after removal from the chronic wound (FIG. 19(c)) (n=4 dressing per wound; mean±st. dev.).

FIGS. 20(a)-(d) is a series of images showing 50 wt % CB-AFTIDerm electrodes unstrained (FIG. 20(a)), stretched (FIG. 20(b)), bended (FIG. 20(c)), and compressed (FIG. 20(d)).

FIG. 21 is a series of SEM images demonstrating the distribution of CB across the AFTIDerm surface.

FIG. 22 is a plot showing water contact angle of CB-AFTIDerm over varied CB concentrations (wt %) (#denotes statistical significance at p<0.05 for 15% and 20%; $ denotes statistical significance at p<0.05 for 15% and 30%; & denotes statistical significance at p<0.05 for 35% and 50%; n=9 measurements per concentration group; mean±st. dev.).

FIGS. 23(a)-(b) are a schematic illustration of the experimental set-up for testing the CB-AFTIDerm electrodes. Kelvin clips were connected near the edge of each sample end at equidistant points 0.5 cm from the edges (FIG. 23(a)). Kelvin clips were vertically connected to measure through thickness impedance of CB-AFTIDerm (FIG. 23(b)).

FIG. 24 is a plot showing current-voltage sweeps at varied CB concentrations (wt %). Data presented as the mean of three samples per CB concentration group. Data represents mean I-V curves for 3 samples.

FIG. 25 is a plot showing resistance profiles at varied CB concentrations (wt %) (n=3 samples per concentration group; mean±st. dev.).

FIG. 26 is a plot showing resistivity profiles at varied CB concentrations (wt %) (n=3 samples per concentration group; mean±st. dev.).

FIGS. 27(a)-(b) are a series of plots showing long-term electrical stability of the CB-AFTIDerm electrodes. Lateral and through thickness behavior of the CB-AFTIDerm electrodes over a 25-hour period (FIG. 27(a)). Temperature output of the CB-AFTIDerm electrodes at hour 0 and hour 25 in both the lateral and through thickness directions (FIG. 27(b)) (n=3 measurements at each timepoint; mean±st. dev.).

FIGS. 28(a)-(b) are a series of plots showing thermal stability of the CB-AFTIDerm electrode based on changes in temperature. Lateral and through thickness resistance versus temperature (FIG. 28(a)). Normalized change in resistance versus temperature (FIG. 28(b)) (n=3 measurements; data represented as mean±st. dev.).

FIGS. 29(a)-(b) are a series of plots showing lateral and through thickness impedance comparing CB-AFTIDerm against AG-735. Lateral direction (FIG. 29(a)) and through thickness direction (FIG. 29(b)). Experiments performed in triplicate (data presented as mean±st. dev.).

FIG. 30 is a schematic of the force gauge used to measure adhesion.

FIGS. 31(a)-(b) are a series of plots showing the assessed relationship between the through thickness impedance and adhesion of the CB-AFTIDerm electrode. Adhesion of conductive tape to a copper electrode, CB-AFTIDerm electrode cured on a copper electrode, and CB-AFTIDerm and conductive tape adhered to the copper electrode (FIG. 31(a)). Through thickness impedance of the conductive tape and CB-AFTIDerm electrode versus that just of the CB-AFTIDerm electrode (FIG. 31(b)). (*denotes p<0.05 compared to 3M XYZ 9713 Tape; #denotes p<0.05 compared to CB-AFTIDerm and 3M XYZ 9713 Tape; $ denotes p<0.05 compared to 3M XYZ 9713 Tape). Experiments run in triplicate (data reported as mean±st. dev.).

FIG. 32 is a plot showing the results of a 7-day absorption study comparing the 50 wt % CB-AFTIDerm electrode versus AG 735 hydrogel electrode (n=3 samples; mean±st. dev.).

FIGS. 33(a)-(b) are a series of plots showing the assessed change in pH of samples over a one-week period. Change in pH measurements over a one-week period (FIG. 33(a)). Change in pH when normalized to PBS (FIG. 33(b)). Experiments run in triplicate (data reported as mean±st. dev.).

FIG. 34 is a schematic showing a composite comprising AFTIDerm and CB-AFTIDerm.

FIG. 35 is a plot comparing mass increase (%) versus days of the 50 wt % CB-AFTIDerm composite and Telfa. Experiments run in triplicate (data reported as mean±st. dev.).

FIG. 36 is a plot showing normalized mass increase per volume of the tested material (e.g., 50 wt % CB-AFTIDerm composite or Telfa). Experiments run in triplicate (data reported as mean±st. dev.).

FIGS. 37(a)-(e) show the results of clinical testing of the CB-AFTIDerm composite on a Yorkshire pig model over a 35 day period. Image of the CB-AFTIDerm composite at Day 17 when placed on a wound of the pig and removal of the composite at Day 21 (FIG. 37(a)). Images of the wound over a one-week period to evaluate skin conditions following administration and removal of the CB-AFTIDerm composite and standard of care wound dressing (FIG. 37(b)). Change in wound temperature over the 35-day of the CB-AFTIDerm composite and standard of care Telfa wound dressing (FIG. 37(c)). Change in wound pH over the 35-day of the CB-AFTIDerm composite and standard of care Telfa wound dressing (FIG. 37(d)). Bright field (top 2 rows) and infrared thermograph (bottom two rows) of CB-AFTIDerm composite and standard of care Telfa dressing taken at weekly time points over the 35-day period (FIG. 37(e)).

FIG. 38 is a plot showing change in temperature between the CB-AFTIDerm composite and the standard of care.

FIG. 39 shows the process-flow for fabrication and integration of a flexible substrate with an elastomeric nanocomposite (referred to below as “Flexatrode”) and AFTIDerm resulted in a multi-material substrate, referred to below as “exciflex”.

FIG. 40 is a schematic showing process-flow for substrate fabrication.

FIG. 41 is an image showing visual inspection of the traces and contact pads for temperature sensing.

FIGS. 42(a)-(d) are a series of microscopy images of the substrate following fabrication using the gel photoresist (electrode contact pad, FIG. 42(a), temperature sensor contact pad (required spacing between contact pad and trace not existent), FIG. 42(b), traces leading to temperature sensor (lack of trace fidelity noted by white arrow), FIG. 42(c), and trace fracture noted by white arrows, FIG. 42(d)).

FIGS. 43(a)-(d) are a series of microscopy images of the substrate following fabrication using the dry film photoresist. Contact pad to PCB (FIG. 43(a)), temperature sensor contact pad (required spacing between contact pad and trace not existent) (FIG. 43(b)), traces leading to temperature sensor (lack of trace fidelity noted by white arrow) (FIG. 43(c)), and traces leading to wound temperature sensor (FIG. 43(d)).

FIGS. 44(a)-(c) are a series of images showing integration of SMT components on the Cu—PI substrate. Deposition of silver epoxy on and next to the copper contact pad (FIG. 44(a)). Microscopy image of the placed temperature sensor (FIG. 44(b)). Fabricated substrate with integrated temperature sensors and capacitors (FIG. 44(c)).

FIG. 45 is a schematic detailing integration of Flexatrode onto copper electrode as a device.

FIG. 46 is a plot showing adhesion testing on a Cu-electrode. Experiments performed in triplicate (mean±st. dev. reported) (denotes statistical significance (p<0.05) between CB-PDMS and CB-PDMS and 3M XYZ 9713 Tape).

FIG. 47 is a plot showing through thickness impedance of Flexatrode, tape, and copper (device integrated in panel an over a one-week period (Day 0: dry, Day 1: hydrated, Day 7: hydrated).

FIG. 48 is a plot showing through thickness impedance of Flexatrode over a one-week period (Day 0: dry, Day 1: hydrated, Day 7: hydrated).

FIG. 49 is a plot showing through thickness impedance of the conductive tape over a one-week period (Day 0: dry, Day 1: hydrated, Day 7: hydrated).

FIGS. 50(a)-(c) are a series of schematics showing exciflex bandages for 6 cm wounds: exciflex 1.0 (FIG. 50(a)); exciflex 2.0 (FIG. 50(b)); and exciflex 3.0 (FIG. 50(c)).

FIG. 51 is a schematic showing the layout of exciflex 1.0.

FIG. 52 is an image showing the initial integration of electronics module with flexible substrate.

FIG. 53 is a series of images showing revised integration of the electronics module with flexible substrate in FIG. 52.

FIG. 54 is a series of schematics showing exciflex 2.0 at various sizes to factor in wound reepithelization.

FIGS. 55(a)-(b) are a series of schematics of exciflex 2.0 6 cm bandage. Electronics side of the bandage (FIG. 55(a)). Skin-facing side of the bandage (FIG. 55(b)).

FIG. 56 is a schematic showing integration of TMP-117 sensors onto the bandage for temperature monitoring.

FIG. 57 is an image showing the integrated exciflex device prior to ethylene oxide sterilization.

FIG. 58 is a series of schematics showing layouts of the exciflex 3.0 bandage sizes.

FIG. 59 is a schematic of an integrated exciflex 3.0 6 cm bandage.

FIG. 60 is a series of images showing exciflex 3.0 4 cm substrates.

FIG. 61 is a plot showing comparative analysis (Ω/μm) of AG-735, Flexatrode, and CB-AFTIDerm (n=3 per sample; data reported as mean±st. dev.).

FIG. 62 is a schematic showing exciflex 3.0 6 cm bandage with CB-AFTIDerm electrodes.

FIG. 63 is a series of images showing pre-clinical process flow from wound creation to wound monitoring.

FIG. 64 is a series of images showing change in wound re-epithelization over a 28-day period. Euthanizing of the animal occurred on Day 35. Images presented are from one pig as an example.

FIG. 65 is a plot showing change in wound area (in cm²) over time.

FIG. 66 is a plot showing change in wound closure (%) over time.

FIG. 67 is a plot showing change in pH over time.

FIG. 68 is a plot showing difference in pH between treatment groups over time.

FIG. 69 is a series of infrared thermograms over a 28-day period. Euthanizing of the animal occurred on Day 35. Images presented are from one pig as an example.

FIG. 70 is a plot showing change in temperature (° C.) over time.

FIG. 71 is a schematic showing the fabrication scheme of hydrogels and organohydrogels as described by Gu et al. (ACS Appl. Mater. Interfaces 2020, 12, 36, 40815-40827) (“Gu”).

FIG. 72 is a series of plots showing hardness (upper plot) and Young's Modulus values (MPa) (lower plot) for the compositions of Gu.

FIG. 73 is a plot showing conductivity (S/m) for the compositions of Gu.

### DETAILED DESCRIPTION

### Definitions

Unless defined otherwise, all technical and scientific terms used herein have the same meaning as is commonly understood by one of skill in the art to which the present disclosure pertains.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

In the context of the present disclosure, the term “about”, when expressed as from “about” one particular value and/or “about” another particular value, also specifically contemplated and disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these aspects are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

As used herein, phrases such as “between X and Y” and “between about X and Y” can be interpreted to include X and Y.

As used herein, phrases such as “between about X and Y” can mean “between about X and about Y”.

As used herein, phrases such as “from about X to Y” can mean “from about X to about Y”.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms can encompass different orientations of the device in use or operation in addition to the orientation depicted in the figures. For example, if the device in the figures is inverted, elements described as “under” or “beneath” other elements or features would then be oriented “over” the other elements or features.

As used herein, the term “acute wound” can refer to a wound caused by a traumatic abrasion, burn, laceration or through superficial damage.

As used herein, the term “and/or” can include any and all combinations of one or more of the associated listed items.

As used herein, the term “AFTIDerm” can refer to an absorbent, flexible, and transparent substrate that can be used, for example, as a wound dressing and in various bioelectronic applications. In one example, AFTIDerm can include a moisture absorbing layer that comprises a thermoplastic material (e.g., PVA) and a polyol compound (e.g., glycerol), wherein the moisture absorbing layer is formulated to absorb moisture (e.g., from a wound site) but without swelling. Other examples of AFTIDerm are described throughout the present application, including in the Examples.

As used herein, the term “carbon black” or “CB” can refer to any of a group of intensely black, finely divided forms of amorphous carbon. Carbon black particles are usually spherical in shape and less regularly crystalline than graphite. Carbon black can vary widely in particle size depending on the process by which it is made (e.g., channel or impingement black, furnace black, thermal black, acetylene black).

As used herein, the term “CB-AFTIDerm” can refer to an absorbent, flexible, and electrically-conductive substrate that can be used, for example, in various bioelectronic applications, such as wearable electronics and electroceuticals. In one example, CB-AFTIDerm can comprise carbon black, a thermoplastic material (e.g., PVA), and a polyol compound (e.g., glycerol), wherein the CB-AFTIDerm is formulated to absorb moisture (e.g., liquid/exudate from a wound site or eccrine sweat) but without swelling. Other examples of CB-AFTIDerm are described throughout the present application, including in the Examples.

As used herein, the term “chronic wound” can refer to a wound in which there is no clot information, normally occurring in patients who are compromised in some fashion who are less likely to heal. When the body's natural healing process is delayed due to an underlying pathological process, such as vascular in sufficiency, it may lead to a chronic wound. The term can also refer to a category of wound that fails to heal over a typical (e.g., 8-12 weeks) timeframe from inception of the wound to complete closure of the skin at the wound site.

As used herein, the term “bioactive agent” can refer to any agent, compound, drug, substance, or moiety that promotes wound healing processes over days, weeks, or months. Non-limiting examples of bioactive agents can include antibodies (e.g., that specifically binds to ICAMs, VCAMs, PECAMs or ELAMs), extracellular matrix proteins (e.g., glycosaminoglycans, proteoglycans, collagen, elastin, fibronectin, laminin, alginate, a chitin derivative), proteinaceous growth factors (e.g., PDGF-BB, TNF-alpha, EGF, KGF, VEGFs, FGFs, TNF-beta and IGF-1), antimicrobial agents (e.g., antibiotics), and steroids.

As used herein, the term “electrical communication” can refer to certain parts, components, or features that are in communication with each other by flow of electrons through conductors, such as wires or circuitry. The term can also include electrochemical communication that involves flow of ions, such as Li⁺, through electrolytes. In some instances, the term can also include wireless communication.

As used herein, the term “electroceutical device” can refer to any medical device that provides neurostimulation for therapy.

As used herein, the term “exciflex” can refer to electroceutical devices constructed in accordance with various aspects of the present disclosure. In one example, the term can include an electroceutical device comprising: a moisture absorbing layer having a wound side and an opposed outer side; a plurality of electrodes disposed over the wound side of the moisture absorbing layer; an occlusive layer positioned against the outer side of the moisture absorbing layer, wherein the occlusive layer comprises flexible circuitry that defines a plurality of electrical contacts; and a plurality of temperature sensors coupled to the flexible circuitry; wherein each electrode of the plurality of electrodes is in electrical communication with a respective contact of the plurality of contacts of the flexible circuitry. Other examples of exciflex are described throughout the present application, including in the Examples.

As used herein, the term “exogenous conductive element” can refer to any conductive element that is added to a moisture absorbing layer of the present disclosure to enhance, increase, or improve the electrical conductivity of the moisture absorbing layer beyond or in addition to a degree of electrical conductivity that may exist in the moisture absorbing layer without the addition of the conductive element. Non-limiting examples of conductive elements can include salts (e.g., NaCl), carbon nanotubes, silver nanowires, metal particles, eutectic gallium-indium alloy, graphite flakes, and the like.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “Flexitrode” can refer to an elastomeric nanocomposite as disclosed in PCT App. No. PCT/US2021/26571, filed Apr. 9, 2021, entitled “Flexible nonmetallic electrode”. In one example, the term can refer to a flexible, nonmetallic electrode that comprises, consists essentially of, or consists of carbon black and a polymer (e.g., PDMS or PVA), where the carbon black is provided at a weight of between 10% and 50% of a weight of the polymer (e.g., the weight of the carbon black is about 25% of the weight of the PDMS or about 50% of the weight of the PVA).

As used herein, the terms “heal” or “healing”, when used in the context of a wound site, can refer to the biological process whereby a wound site progresses through the three steps of wound healing: removal of necrotic and nonvital material (autolytic debridement) by inflammation (e.g., macrophages); neovascular growth; and proliferation of dermal/epidermal cells. The degree to which a wound site is healed or healing can be assessed based on one or a combination of wound characteristics, such as changes in wound area over time, changes in wound closure over time, changes in pH over time, and changes in temperature over time.

As used herein, the terms “optionally” and “optional” can mean that the subsequently described event, circumstance, or material may or may not occur or be present, and that the description includes instances where the event, circumstance, or material occurs or is present and instances where it does not occur or is not present.

As used herein, the term “polyol compound” can refer to an organic compound containing multiple hydroxyl groups, e.g., monosaccharide and disaccharide molecules in which the aldehyde group is replaced by a hydroxyl. One example of a polyol compound is glycerol.

As used herein, the term “subject” can refer to a vertebrate, such as a mammal (e.g., a human). Mammals can include, but are not limited to, humans, dogs, cats, horses, cows, and pigs.

As used herein, the term “thermoplastic material” can refer to a polymeric material that can be melted and recast almost indefinitely. Thermoplastic materials are molten when heated and harden upon cooling. Non-limiting examples of thermoplastic materials include polypropylene, polyethylene, polyvinylchloride, polystyrene, polyethyleneterephthalate and polycarbonate. One example of a thermoplastic material is poly(vinyl alcohol) or PVA.

As used herein, the term “via” can refer to a vertical interconnect access (via) structure or component as is known in the art.

As used herein, the term “weight percent”, represented as “wt %”, can refer to a ratio or proportion of a first substance to a second substance, and can be understood as a weight/mass of the first substance as a percentage of a weight/mass of the second substance (i.e., a ratio of the weight or mass of the first substance to the weight or mass of the second substance, expressed as a percentage). Thus, for example, one gram of carbon black combined with ten grams of a composite (e.g., PVA and glycerol) can have a carbon black weight percent of 10%.

As used herein, the term “wound site” can refer to a break in the continuity of the skin barrier that may result from one or a combination of causes, such as trauma, surgery, infection, prolonged surface pressure, etc. The term can include can partial and full thickness wounds. A partial thickness wound can refer to a wound that is limited to the epidermis and superficial dermis with no damage to the dermal blood vessels. A full thickness wound can refer to a wound that involves total loss of epidermal and dermal layers of the skin, extending at least to the subcutaneous tissue layer and possibly as deep as the fascia-muscle layer and the bone.

As used herein, the term “wireless communication” can refer to any of various kinds of communication in which information is exchanged without the use of wires. For example, wireless communication may involve transmitting data using available parts of the electromagnetic spectrum, such as infrared radiation, microwaves, or radio waves. The term can also involve wireless power transfer between electrical components.

### Overview

The present disclosure relates generally to transparent, flexible substrates and, in particular, moisture-absorbing substrates for use in wound healing and wearable bioelectronics. For ease of reference, the present application is organized as follows:
Section (I) describes a wound dressing constructed in accordance with one aspect of the present disclosure;
Section (II) describes devices constructed in accordance with another aspect of the present disclosure and comprising the wound dressing in Section (I) for monitoring and/or treating wound sites;
Section (III) describes methods according to another aspect of the present disclosure for monitoring and/or treating wound sites using the wound dressing of Section (I) as well as the devices of Section (III);
Section (IV) describes electrodes constructed in accordance with another aspect of the present disclosure;
Section (V) describes devices constructed in accordance with another aspect of the present disclosure comprising the electrode in Section (IV) for a wide range of applications; and
Section (VI) describes methods according to another aspect of the present disclosure for using the electrodes in Section (IV) as well as the devices in Section (V) for a wide range of applications.

### I

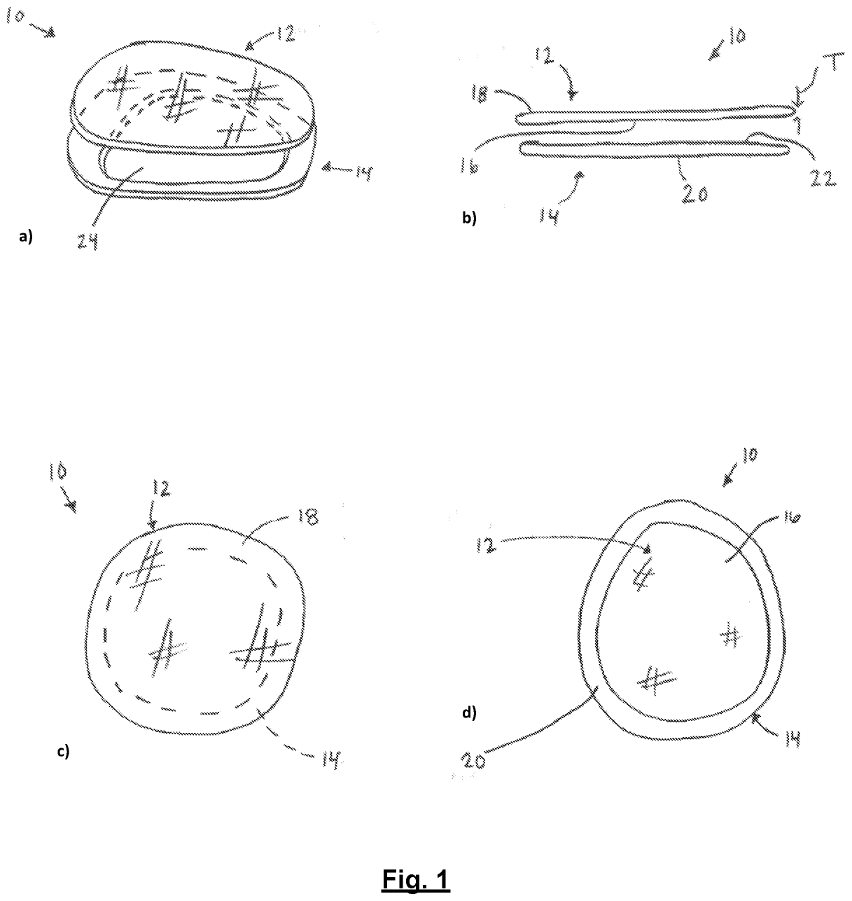

One aspect of the present disclosure can include a wound dressing 10 (FIGS. 1A-D) for application against a wound site of a subject. The wound dressing 10 can comprise a transparent, moisture absorbing layer 12 and an adhesive layer 14. The moisture absorbing layer 12 can have a wound side 16 and an opposed outer side 18. The adhesive layer 14 can be connected to at least a portion of the wound side 16 of the moisture absorbing layer 12. The adhesive layer 14 can facilitate attachment of the wound dressing 10 to a non-wounded perimeter of the wound site. The moisture absorbing layer 12 can absorb moisture from the wound site, without swelling, to promote healing of the wound site.

As discussed herein, the moisture absorbing layer 12 of the present disclosure imparts wound dressings with several advantages over conventional wound dressings, such as those listed in Table 1 above. It was surprisingly found by the inventors, for instance, that a moisture absorbing layer 12 constructed in accordance with one aspect of the present disclosure was stretchable and remained intact under torsion (demonstrating long-term mechanical stability), exhibited negligible absorption drop, and demonstrated an increase in absorption without swelling. As such, the moisture absorbing layer 12 can absorb moisture (e.g., wound exudate, apocrine sweat, eccrine sweat) while remaining transparent without swelling. These features permit application of the moisture absorbing layer 12 (and thus a wound dressing 10) to a wound site for extended periods of time (e.g., up to 7 days) without the need to change the dressing while also permitting observation of the wound site despite moisture (e.g., wound exudate) absorption by the moisture absorbing layer.

In one aspect, the moisture absorbing layer 12 can comprise a thermoplastic material and a polyol compound. The concentration of the thermoplastic material in the moisture absorbing layer 12 is such that the moisture absorbing layer 12 remains flexible under torsion while also retaining its hydrophilicity. In some instances, the concentration of the thermoplastic material can be about 1 wt % to about 10%, about 2 wt % to about 9 wt %, about 3 wt % to about 8 wt %, about 4 wt % to about 7 wt %, or about 5 wt % to about 6 wt %.

In one example, the thermoplastic material is poly(vinyl alcohol) (PVA). The concentration of the PVA in the moisture absorbing layer 12 can be about 1 wt % to about 10%, about 2 wt % to about 9 wt %, about 3 wt % to about 8 wt %, about 4 wt % to about 7 wt %, or about 5 wt % to about 6 wt %. In another example, the concentration of the PVA in the moisture absorbing layer 12 can be about 1 wt % to about 5 wt %. In a further example, the concentration of the PVA in the moisture absorbing layer 12 can be about 3 wt %.

In another aspect, the moisture absorbing layer 12 can include an amount of a polyol compound having a concentration that imparts the moisture absorbing layer 12 with thermoplasticity, self-healing, and long-term moisture retention while also increasing its low-temperature tolerance. In some instances, the concentration of the polyol compound can be about 3 wt % to about 15 wt %, about 4 wt % to about 14 wt %, about 5 wt % to about 13 wt %, about 6 wt % to about 12 wt %, about 7 wt % to about 11 wt %, or about 8 wt % to about 10 wt %.

In one example, the polyol compound is glycerol. Advantageously, glycerol can supply multiple hydroxyl groups and, thus, serve as a cross-linker for thermoplastic polymer chains (e.g., PVA) to improve the strength and toughness of hydrogels (e.g., PVA hydrogels). In some instances, the concentration of glycerol in the moisture absorbing layer 12 can be about 3 wt % to about 15 wt %, about 4 wt % to about 14 wt %, about 5 wt % to about 13 wt %, about 6 wt % to about 12 wt %, about 7 wt % to about 11 wt %, or about 8 wt % to about 10 wt %. In one example, the concentration of glycerol in the moisture absorbing layer 12 can be about 3 wt % to about 15 wt %, e.g., about 5 wt % to about 10 wt %, e.g., about 5 wt %. As discussed in Example 1 below, the inventors surprisingly discovered that introduction of glycerol (e.g., at 5 wt %) into a PVA hydrogel provided a moisture absorbing layer that was stretchable, remained intact under torsion, exhibited thermoplasticity, self-healing, and long-term moisture retention while also increasing its low-temperature tolerance.

In one example, the moisture absorbing 12 can comprise about 3 wt % PVA and about 5 wt % glycerol.

In another aspect, the moisture absorbing layer 12 can remain transparent after absorbing moisture, such as wound exudate, apocrine sweat or eccrine sweat. The fact that the moisture absorbing layer 12 can remain transparent after absorbing moisture is advantageous as it permits observation of a wound site while the wound dressing 10 is applied thereto. Repeated changing of wound dressings is problematic as it is often painful and removes new cell and tissue layers being formed over a wound site. Wound dressings 10 of the present disclosure can eliminate or significantly reduce unnecessary wound dressing changes as wound site healing can be directly visualized through the moisture absorbing layer 12.

In another aspect, the moisture absorbing layer 12 can absorb moisture (e.g., from a wound site), without swelling, for a period of time following contact of the wound dressing 10 with a wound site. In some instances, the period of time is about 1 hour to about 14 days, or about 24 hours to about 14 days, or about 2 days to about 14 days, or about 4 days to about 14 days, or about 6 days to about 14 days, or about 8 days to about 14 days, or about 10 days to about 14 days, or about 12 days to about 14 days. In one example, the period of time is about 5 days to about 14 days, or about 7 days to about 14 days, or about 7 days. Unlike conventional wound dressings, the moisture absorbing layer 12 can retain its mechanical, thermal, absorption, and biological properties (as discussed herein) over a long period of time (e.g., 7 days), which can accelerate wound healing and decrease costs typically associated with repeated wound dressing changes.

In another aspect, the moisture absorbing layer 12 can include one or more bioactive agents for delivery into tissue comprising the wound site or a surrounding non-wound site. In some instances, the moisture absorbing layer 12 can be formulated with one or more bioactive agents so that the bioactive agent(s) have a desired release or elution profile (e.g., a slow-release profile). Methods for formulating hydrogels with bioactive agents having desired release profiles are known in the art.

In another aspect, the moisture absorbing layer 12 can be free of any exogenous conductive elements. This means that, during fabrication of the moisture absorbing layer 12, and even thereafter, no conductive element or elements is/are added to the moisture absorbing layer.

It will be appreciated that the dimensions and shape of the moisture absorbing layer 12 can be tailored for any given application, e.g., to completely or partially cover a wound site when applied thereto. In some instances, for example, the moisture absorbing layer 12 can have a regular shape (e.g., circle, rectangle, square) or an irregular shape. In some aspects, the moisture absorbing layer 12 has a thickness T (FIG. 1B), which can be defined as the distance between the opposed outer side 18 and the wound side 16 of the moisture absorbing layer. The thickness T of the moisture absorbing layer 12 can be selected to provide the moisture absorbing layer with flexibility sufficient to conform to the irregular topography of the stratum corneum. In one example, the moisture absorbing layer 12 can have a thickness T of between about 10 microns and about 10 mm, or between 50 microns and 200 microns, or about 100 microns.

In another aspect, the wound dressing 10 can comprise an adhesive layer 14 that is configured to attach to the skin of a subject. For example, the adhesive layer 14 can facilitate attachment of the wound dressing 10 to a non-wounded perimeter of the wound site. In one example, the adhesive layer 14 can be comprised of a medical-grade silicone acrylate adhesive. As shown in FIG. 1B, the adhesive layer 14 can include a skin-contacting surface 20 and an upper surface 22 for contact with (and attachment to) the moisture absorbing layer 12. As such, the adhesive layer 14 can be connected to at least a portion of the wound side 16 of the moisture absorbing layer 12.

It will be appreciated that the dimensions and shape of the adhesive layer 14 can be tailored for any given application, e.g., to permit attachment of the wound dressing 10 to a non-wounded perimeter of the wound site. In some instances, for example, the adhesive layer 14 can have a shape and dimensions that mirror (or substantially mirror) the shape and dimensions of the moisture absorbing layer 12. As such, the adhesive layer 14 can have a regular shape (e.g., circle, rectangle, square) or an irregular shape. In one example, as shown in FIGS. 1A-D, the adhesive layer 14 is ring-shaped and can be adhered to an outer perimeter of the wound side 16 of the moisture absorbing layer 12. This configuration of the adhesive layer 14 is advantageous because the central aperture 24 of the adhesive layer permits visualization (through the transparent moisture absorbing layer 12) of the wound site.

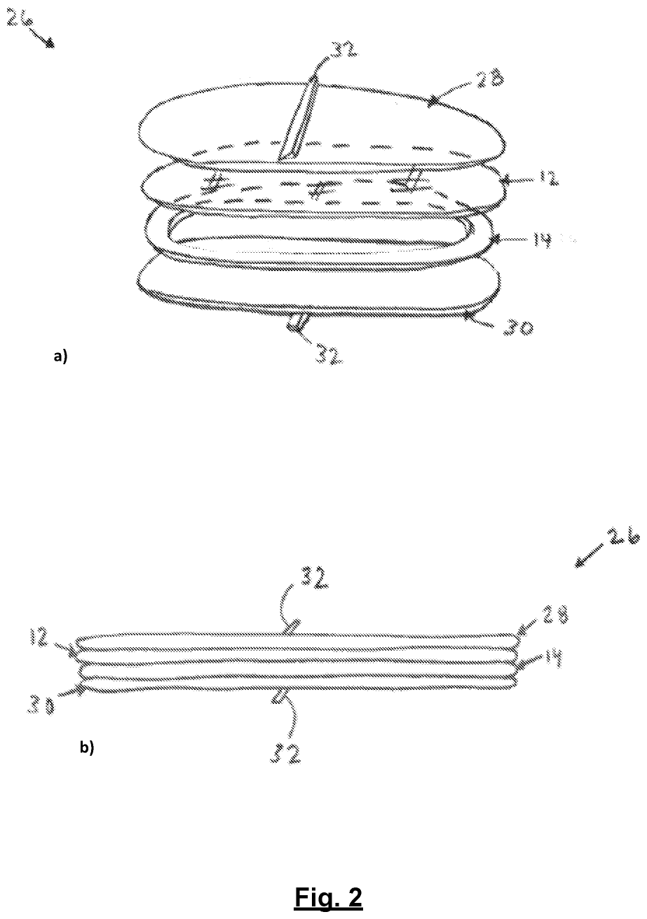

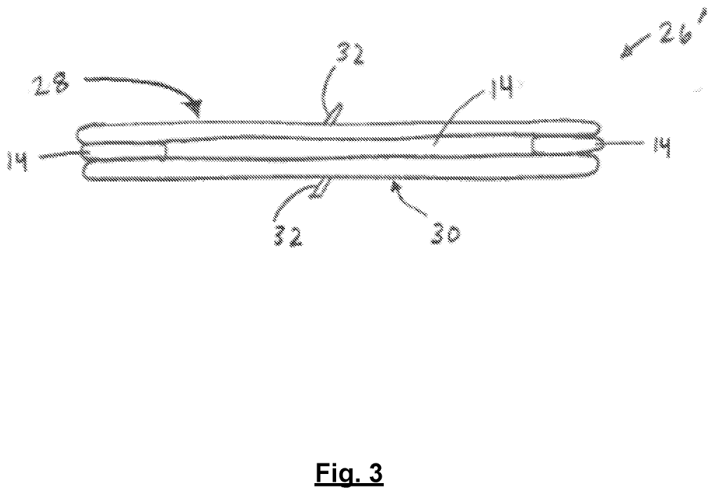

FIGS. 2A-B illustrate one example of a wound dressing assembly 26 constructed in accordance with an aspect of the present disclosure. In some instances, the wound dressing assembly 26 can comprise a wound dressing 10 as described above and shown in FIGS. 1A-D. The wound dressing assembly 26 can further comprise first and second backing layers 28 and 30 to protect the wound dressing 10 prior to application. The first backing layer 28 can be disposed over the opposed outer side 18 of the moisture absorbing layer 12, and the second backing layer 30 can be disposed over the skin contacting surface 20 of the adhesive layer 14. In one example, each of the first and second backing layers 28 and 30 can comprise a flexible polymer substrate. Each of the first and second backing layers 28 and 30 can include a tab 32 to facilitate removal of the first and second backing layers from the wound dressing 10. It will be appreciated that other configurations of the wound dressing assembly 26 are possible. As shown in FIG. 3, for example, a wound dressing assembly 26′ can be configured such that the moisture absorbing layer 12 is seated within the central aperture 24 of the adhesive layer 14. In this configuration, the first backing layer 28 is in direct contact with the opposed outer side 18 of the moisture absorbing layer 12 as well as the upper surface 22 of the adhesive layer 14. Further, the second backing layer 30 is in direct contact with the wound side 16 of the moisture absorbing layer 12 and the skin contacting surface 20 of the adhesive layer 14.

### II

Another aspect of the present disclosure includes a device for monitoring and/or healing a wound site of a subject. A device for monitoring and/or healing a wound site can comprise a transparent, moisture absorbing layer, an occlusive layer, and a plurality of temperature sensors. It is known that temperature changes in different types and stages of wounds are closely related to the wound healing status. Advantageously, such devices incorporate the features of the moisture absorbing layer (discussed above) along with the ability to monitor, in real-time, temperature changes associated with a wound site. As such, devices of the present disclosure permit quantitative measurement of temperature to assist with objective, rapid, and easy-to-interpret assessment of wound healing status.

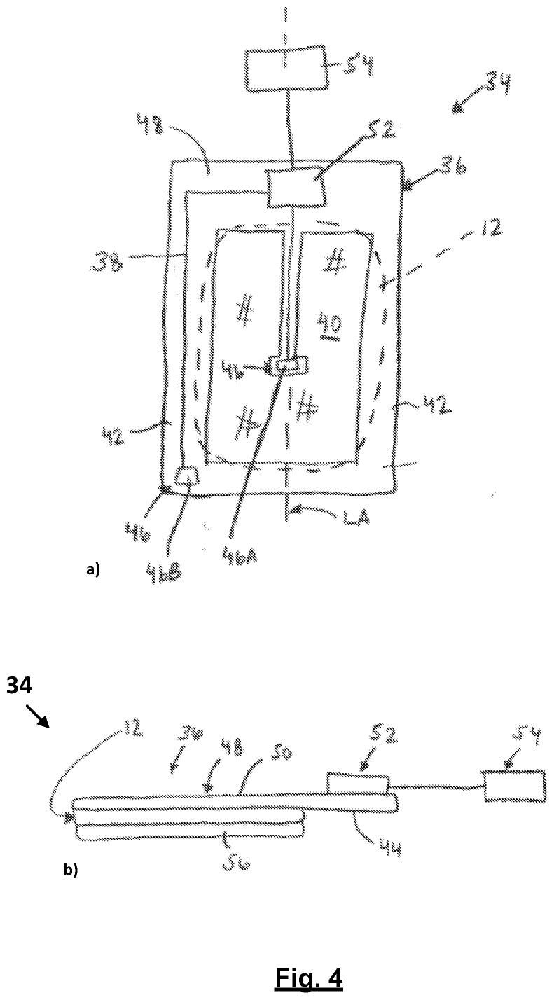

One example of a device 34 for monitoring and/or healing a wound site is shown in FIGS. 4A-B. The device 34 can comprise a transparent, moisture absorbing layer 12 having a wound side 16 and an opposed outer side 18. The moisture absorbing layer 12 can be prepared as discussed in Section (I) above.

The device 34 can additionally comprise an occlusive layer 36 positioned against at least a portion of the outer side 18 of the moisture absorbing layer 12. The occlusive layer 36 can comprise flexible circuitry 38 (e.g., copper traces) that define a plurality of electrical contacts (not shown). The occlusive layer 36 can comprise a flexible, transparent window 40 and a flexible adhesion portion 42 that extends around the circumference of the transparent window and is configured to adhere to the skin of a subject to enclose the moisture absorbing layer 12 between the occlusive layer and the subject. The transparent window 42 can comprise a flexible biocompatible polymeric material, such as SYLGARD polymer (manufactured by DOW), optionally, with moisture absorbent additives included therein. The flexible adhesion portion 42 can serve to seal against the skin and prevent wound exudate from escaping. Optionally, the flexible adhesion portion 42 and the transparent window 40 can be provided as a single, integral structure. Advantageously, the moisture absorbing layer 12, in cooperation with the transparent window 40, provides continued visibility of the wound site.

In one aspect, the flexible circuitry 38 of the device 34 can comprise a copper (Cu)-clad flex-electronics polyimide (or other suitable biocompatible) sheet. Photolithographic patterning can be used to fabricate Cu contact pads for coupling to electrodes (when present; discussed below) on a wound (bottom) side 44 of the occlusive layer 36 as well as interconnect traces for communicating electrical current to and from electrodes and temperature sensors. Via trenches for vertical interconnects between flexible circuitry 38 on the two sides of the occlusive layer 36 can be defined by laser micromachining and can be filled by Cu electroplating to define the vias.

The device 34 further includes a plurality of temperature sensors 46 (optionally, a first temperature sensor 46A and a second temperature sensor 46B) coupled to (e.g., in electrical communication with) the flexible circuitry 38. In one example, the plurality of temperature sensors 46 is located on an upper side 48 of the occlusive layer 36. As shown in FIG. 4A, for example, a first temperature sensor 46A can be located on the upper side 48 of the occlusive layer 36 so that the first temperature sensor is positioned directly above the window 40 (e.g., so the first temperature sensor is positioned above a wound site when the device 34 is applied thereto). As also shown in FIG. 4A, the second temperature sensor 46B is located on an upper surface 50 of the flexible adhesion portion 42 that extends around the circumference of the transparent window 40 so that the second temperature sensor is spaced a small distance from the wound site and located above a non-wounded perimeter of the wound site when the device 34 is applied thereto.

In some instances, the first temperature sensor 46A can be positioned over or within the wound bed, while the second temperature sensor 46B can be positioned away from (i.e., depending on the orientation of the wound and the device 34, laterally or vertically spaced from) the wound bed. For example, when the wound bed is oriented horizontally, it is contemplated that the second temperature sensor 46B can be sufficiently horizontally spaced from the wound bed so that the temperature measured by the second temperature sensor reflects ambient/systemic temperature information (rather than the temperature at or within the wound). As shown in FIG. 4A, the device 34 can have a longitudinal axis LA. Optionally, the first and second temperature sensors 46A and 46B can be aligned along the longitudinal axis LA.

In some instances, the first and second temperature sensors 46A and 46B can be spaced apart from each other by at least 1.5 centimeters, at least two centimeters, or at least four centimeters (e.g., from about two centimeters to about three centimeters, from about three centimeters to about four centimeters, from about four centimeters to about five centimeters, from about five centimeters to about six centimeters, from about six centimeters to about seven centimeters, from about seven centimeters to about eight centimeters, from about eight centimeters to about nine centimeters, from about nine centimeters to about ten centimeters, from about ten centimeters to about twelve centimeters, or more.

In some instances, the moisture absorbing layer 12 can optionally define holes (not shown) therethrough, and the temperature sensors 46 can be positioned within the holes and attached to the wound side 44 of the occlusive layer 36. Alternatively, one or more temperature sensors 46 can be attached (e.g., directly attached) to the wound side 44 of the occlusive layer 36. In such instances, it will be appreciated that a respective copper (or other thermally conductive material) contact pad (not shown) can be positioned against the skin of the subject and can interface between each of the temperature sensors 46 and the skin. Thermal diffusion from the skin can be relayed through the contact pads.

In one example, the temperature sensor(s) 46 (optionally, first and second temperature sensors 46A and 46B) can comprise a TMP-117 (e.g., 2 mm×2 mm) sensor. The TMP-117 is a high-precision digital temperature sensor designed to meet ASTM E1112 and ISO 80601 requirements for electronic patient thermometers. The TMP-117 provides a 16-bit temperature result with a resolution of 0.0078° C. and an accuracy of up to ±0.1° C. (clinical standard) across the temperature range of −20° C. to 50° C. with no calibration. The TMP-117 operates from 1.7 V to 5.5 V and consumes ˜3.5 μA. The low power consumption of the TMP-117 minimizes the impact of self-heating on measurement accuracy. TMP-117 operates as a pyroelectric sensor which converts the heat radiated from the skin to a voltage output to the board and a temperature value on a corresponding application platform.

In another aspect, the device 34 can include a control module 52 in electrical communication with the plurality of temperature sensors 46. The control module 52 can be operative to receive and/or store a signal from each temperature sensor 46 of the plurality of temperature sensors. In some instances, where the plurality of temperature sensors 46 comprises a first temperature sensor 46A and a second temperature sensor 46B, the control module 52 can be programmed or operative to receive a temperature measurement from between the first temperature sensor and the second temperature sensor, and then transmit, to a remote device 54, a signal corresponding to the temperature measurement.

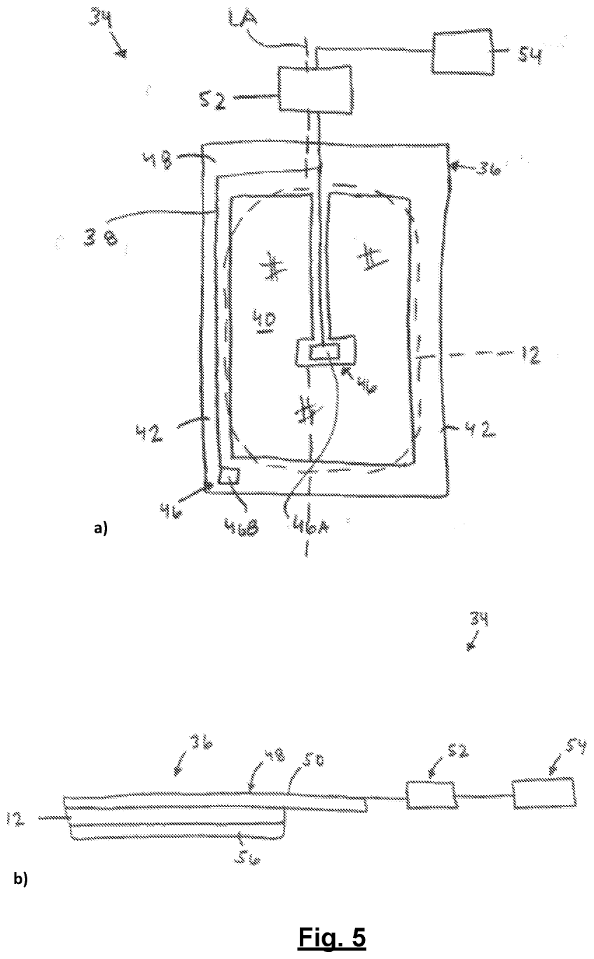

As shown in FIGS. 4A-B, the control module 52 can be located on the upper surface 50 of the occlusive layer 36 and be in electrical communication with the remote device 54, which is physically spaced apart from, and not located on, the occlusive layer. Optionally, in an alternative configuration shown in FIGS. 5A-B, the control module 52 can be physically spaced apart from the occlusive layer 36 (e.g., not physically located on the occlusive layer), but still remain in electrical communication with the flexible circuitry 38 of the device 34.

The control module 52 and/or the remote device 54 can be provided as a computing device. As such, a computing device according to an aspect of the present disclosure is described below. The computing device can perform various aspects of monitoring temperature readings from the plurality of temperature sensors 46. The computing device may comprise one or more processors, a system memory, and a bus that couples various components of the computing device including the one or more processors to the system memory. In the case of multiple processors, the computing device may utilize parallel computing.

The bus may comprise one or more of several possible types of bus structures, such as a memory bus, memory controller, a peripheral bus, an accelerated graphics port, and a processor or local bus using any of a variety of bus architectures.

The computing device may operate on and/or comprise a variety of computer readable media (e.g., non-transitory). Computer readable media may be any available media that is accessible by the computing device and comprises, non-transitory, volatile and/or non-volatile media, removable and non-removable media. The system memory has computer readable media in the form of volatile memory, such as random access memory (RAM), and/or non-volatile memory, such as read only memory (ROM). The system memory may store data such as wound data and/or program modules such as operating system and wound monitoring software that are accessible to and/or are operated on by the one or more processors.

The computing device may also comprise other removable/non-removable, volatile/non-volatile computer storage media. The mass storage device may provide non-volatile storage of computer code, computer readable instructions, data structures, program modules, and other data for the computing device. The mass storage device may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device. An operating system and wound monitoring software may be stored on the mass storage device. One or more of the operating system and wound monitoring software (or some combination thereof) may comprise program modules and the wound monitoring software. Wound data may also be stored on the mass storage device. Wound data may be stored in any of one or more databases known in the art. The databases may be centralized or distributed across multiple locations within the network.

A user (e.g., a clinician) may enter commands and information into the computing device using an input device (not shown). Such input devices comprise, but are not limited to, a touchscreen (e.g., a touchscreen of a smartphone or tablet), a keyboard, a pointing device (e.g., a computer mouse, remote control), a microphone, a joystick, a scanner, tactile input devices such as gloves, and other body coverings, a motion sensor, a voice recognition device, and the like. These and other input devices may be connected to the one or more processors using a human machine interface that is coupled to the bus, but may be connected by other interface and bus structures, such as a parallel port, game port, an IEEE 1394 Port (also known as a Firewire port), a serial port, network adapter, and/or a universal serial bus (USB).

A display device may also be connected to the bus using an interface, such as a display adapter. It is contemplated that the computing device may have more than one display adapter and the computing device may have more than one display device. A display device may be a monitor, an LCD (Liquid Crystal Display), light emitting diode (LED) display, television, smart lens, smart glass, and/or a projector. In addition to the display device, other output peripheral devices may comprise components such as speakers (not shown) and a printer (not shown) which may be connected to the computing device using Input/Output Interface. Any step and/or result of the methods described herein may be output (or caused to be output) in any form to an output device. Such output may be any form of visual representation, including, but not limited to, textual, graphical, animation, audio, tactile, and the like. The display and computing device may be part of one device, or separate devices.

The control module 52 may operate in a networked environment using logical connections to one or more remote devices 54. A remote device 54 may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the control module 52 and a remote device 54 may be made using a network, such as a local area network (LAN) and/or a general wide area network (WAN). Such network connections may be through a network adapter. A network adapter may be implemented in both wired and wireless environments. Such networking environments are conventional and commonplace in dwellings, offices, enterprise-wide computer networks, intranets, and the Internet. It is contemplated that the remote devices 54 can optionally have some or all of the components disclosed as being part of a control module 52 containing a computing device.

Application programs and other executable program components associated with the control module 52 and/or remote device 54 are executed by the one or more processors of a computing device. For example, an implementation of wound monitoring software may be stored on or sent across some form of computer readable media. Any of the methods disclosed herein may be performed by processor-executable instructions embodied on computer readable media.

Although not shown, it will be appreciated that one or more power sources (e.g., a battery) can be directly or indirectly connected to, and in electrical communication with, the control module 52 and/or the remote device 54. In one example, the control module 52 can physically include a power source, such as a battery. In this way, the device 34 can be portable and can omit cables or wires extending therefrom. Alternatively, it is contemplated that the control module 52 can be electrically coupled to an external power source (for example, using a cord or cable).

In another aspect, the device 34 can include an adhesive patch 56 connected (e.g., directly connected) to the moisture absorbing layer 12 and/or the occlusive layer 36. The adhesive patch 56 can be configured to attach to the skin of a subject, and can be similarly or identically constructed as the adhesive layer 14 (described above).

In another aspect, the device 34 can include one or more impedance sensors (not shown). In some instances, one or more impedance sensors can be adhered to, or located on, the occlusive layer 36 in addition to the temperature sensors 46. In other instances, the temperature sensors 46 can be substituted with impedance sensors. In still further instances, the temperature sensors 46 can be configured to detect temperature as well as impedance.

In another aspect, the device 34 can optionally include a plurality of electrodes (not shown) disposed over the wound side 16 of the moisture absorbing layer 12; or disposed over a wound side 44 of the occlusive layer 36. As described above for the temperature sensors 46, the electrode(s) can be in electrical communication with a respective contact of the plurality of contacts of the flexible circuitry 38. In some instances, the electrodes can be multi-layered, multi-material electrodes. In one example, the electrode(s) can comprise an elastomeric nanocomposite, such as Flexitrode. Operation of the electrode(s) (e.g., for electrical stimulation) can be performed as described below.

### III

Another aspect of the present disclosure can include a method for monitoring healing of a wound site. In one example, the wound site is an acute wound. In another example, the wound site is a chronic wound. One step of the method can include positioned a device 34, as described in Section (II), over a wound site of a subject so that a first temperature sensor 46A is positioned within or over the wound site and a second temperature sensor 46B is positioned at a location spaced apart from the wound site. Next, a status of the wound site can be determined, by a processing device (e.g., a computing device associated with a control module 52 and/or a remote device 54), based on a temperature difference between the first temperature sensor 46A and the second temperature sensor 46B.

In one example, the determined status is an infection status of the wound site.

In another example, the determined status is an ischemic status of the wound site.

The optimal wound bed temperature for healing can be 33° C. However, wound bed temperature can fluctuate greatly due to infection, ischemia or even simply due to dressing changes. For example, the wound bed can have a higher temperature than outside the wound if the wound is infected, and the wound bed can have a lower temperature than outside the wound if the wound is ischemic. Thus, biocompatible temperature sensors 46 of appropriate range and sensitivity can be utilized. Temperature Coefficient of Resistance (TCR) is a material properties parameter used to relate the change in resistance with change of temperature. In exemplary aspects, the temperature sensors 46 disclosed herein can measure a change in resistance that can be converted to a corresponding temperature change using conventional methods (for example, using TCR parameters).

In use, at least one temperature sensor 46 can be located over the wound bed (i.e., the area of the wound), and at least one other temperature sensor can be located over intact periwound skin. The temperature sensor 46 located over the periwound skin (i.e., spaced away from the wound area) can provide ambient/systemic temperature that can provide insight to the local wound microenvironment. In exemplary aspects, it is contemplated that the actual “contact” surface area between each temperature sensor 46 and the subject can range from about one square millimeter to about 200 square millimeters, from about 1.25 square millimeters to about 150 square millimeters, from about 1.5 square millimeters to about 100 square millimeters, from about 1.75 square millimeters to about 25 square millimeters, or from about two square millimeters to about five square millimeters.

Thermal noise can be corrected by subtracting the periwound temperature measurement from the measurement from the temperature sensor 46 located over the wound bed. A temperature sensor 46 can be created by inkjet printing conductive traces on a robust substrate or by other appropriate means of microfabrication. The substrate can be electrically insulating, chemically stable and biocompatible. Some optional materials for the substrate can include liquid crystal polymer, polyimide, parylene, polyethylene terephthalate (PET), polyethylene naphthalate (PEN).

Contact pads on the temperature sensors 46 can be connected to the control module 52 by conducting vias, which can comprise holes or openings that extend through at least a portion of the thickness of the device 34 as further disclosed herein. In use, the temperature sensors 46 can exhibit a linear response within the clinically relevant range of about 35° C. to about 40° C. (about 95° F. to about 104° F.). The temperature sensors 46 can optionally provide accurate temperature measurements to within about 0.1° C. within the clinically relevant range.

In another aspect, the method for monitoring healing of a wound site described above can alternatively or optionally be performed using a device 34 that includes one or more impedance sensors. It is contemplated that changes in wound impedance (i.e., the impedance across the wound) over time can be an indicator of progress of wound closure and healing. For example, an open wound can have an impedance of 1-50, whereas healed human skin can have an impedance of at least an order of magnitude higher and, in some situations, about 10 kΩ. The impedance difference can be primarily due to the stratum corneum. As the wound heals, area impedance of the wound can increase, and an up-turn can occur as re-epithelialization occurs. Moreover, excess moisture in the wound bed due to exudate can substantially lower the impedance across the wound. Thus, measurement of the wound impedance can enable monitoring of both progress of the wound as well as excess moisture accumulation. The impedance between the sensors can be measured over time. In this way, a clinician can remotely monitor the status of a wound site in real-time without disturbing the wound environment.

Another aspect of the present disclosure can include a method for healing a wound site of a subject. One step of the method can include applying a wound dressing 10 (as described in Section (I)) or a device 34 (as described in Section (II)) over the wound site. The wound dressing 10 or the device 34 can then be left over the wound site for a period of time until the wound site is healed. Additionally or optionally, where the device 34 includes one or more electrodes (e.g., made of an elastomeric nanocomposite), a series of electrical stimulations can be applied to the wound site to facilitate healing of the wound site. Further description of electrical stimulation by electroceutical devices of the present disclosure are provided below.

### IV

Another aspect of the present disclosure can include electrodes that may find use in a variety of applications, including wearable electronics and wound monitoring/healing. The development of flexible electrodes has garnered heightened interest by the wearables community for monitoring and treating human health and performance in a non-invasive and unobtrusive manner. Advantageously, the inventors of the present disclosure have discovered a material that absorbs moisture, without swelling, while also providing high flexibility, electrical conductivity, and electrical stability over a long-term duration. Such material, as described below, can be formulated as an electrode for a variety of bioelectronic applications.

Additionally, it can be understood that dry electrodes can create at least three prominent difficulties. First, adhesion of the dry electrode to the stratum corneum (i.e., the outer layer of the skin) with or without the presence of eccrine or apocrine sweat can be difficult. Second, sufficient conductivity of the electrode can be difficult to obtain, particularly in comparison to conventional gel-based electrodes. Third, conventional conductive electrolyte adhesives dry out after a certain period and can usually only be worn for a certain duration (e.g., no more than a few days). Thus, although the conductive adhesive/hydrogels can improve electrical communication, their ability to dry out and cause skin irritation with the stratum corneum can hinder their long-term utility for bio-potential monitoring or healthcare applications which necessitate the use of electrode technology. Advantageously, electrodes disclosed herein address these prominent deficiencies.

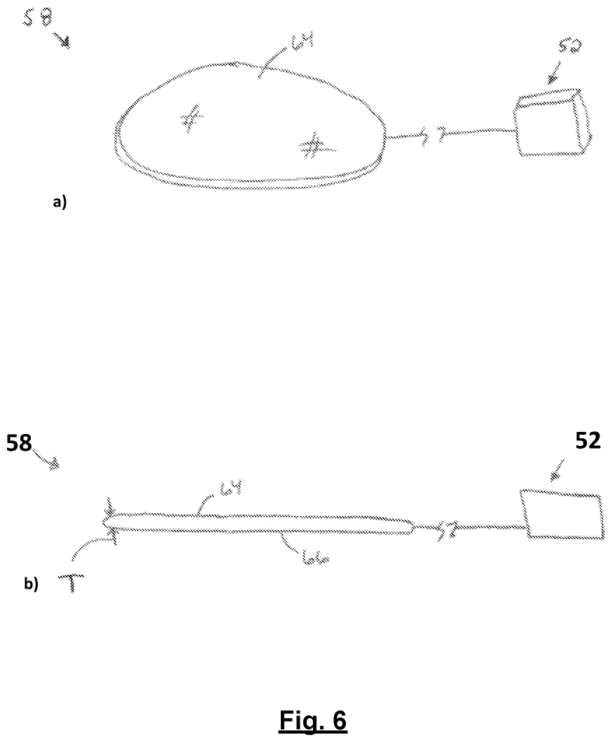

In one aspect, an electrode 58 (FIGS. 6A-B) of the present disclosure can comprise carbon black, a thermoplastic material, and a polyol compound.

In another aspect, an electrode 58 of the present disclosure is a “dry electrode”. That is, electrodes 58 of the present disclosure do not require conductive gels, such as hydrogels. As such, in some instances, an electrode 58 of the present disclosure is physically free from, or is not in physical contact with, a conductive gel (e.g., a hydrogel).

In some instances, the carbon black is provided at a weight of between about 10% and about 70%, between about 15% and about 65%, between about 20% and about 60%, between about 25% and about 55%, between about 30% and about 50%, between about 30% and about 45%, or between about 35% and about 40% of a weight of the thermoplastic material and the polyol compound. In one example, the weight of the carbon black is between about 35 and about 60% of the weight of the thermoplastic material and the polyol compound. In another example, the weight of the carbon black is about 50% of the weight of the thermoplastic material and the polyol compound. In some aspects, the weight percent of carbon black can be selected based on the amount of flexion required for a desired application.

It will be appreciated, in some aspects, that any one or combination of conductive materials, other than carbon black, may be used as part of the electrode. Non-limiting examples of such conductive materials can include carbon nanotubes, silver nanowires, metal particles, eutectic gallium-indium alloy and/or graphite flakes. Thus, it is contemplated that in further aspects, any of the above or like conductive materials, or a mixture thereof, can be used in addition to, or as a partial or complete substitution for, the carbon black.

In some instances, the concentration of the thermoplastic material in the electrode 58 is such that the electrode remains flexible under torsion while also retaining its hydrophilicity. For instance, the concentration of the thermoplastic material can be about 1 wt % to about 10%, about 2 wt % to about 9 wt %, about 3 wt % to about 8 wt %, about 4 wt % to about 7 wt %, or about 5 wt % to about 6 wt %.

In one example, the thermoplastic material is PVA. The concentration of the PVA in the electrode 58 can be about 1 wt % to about 10%, about 2 wt % to about 9 wt %, about 3 wt % to about 8 wt %, about 4 wt % to about 7 wt %, or about 5 wt % to about 6 wt %. In another example, the concentration of the PVA in the electrode 58 can be about 1 wt % to about 5 wt %. In a further example, the concentration of the PVA in the electrode can be about 3 wt %.

In some instances, the polyol compound can have a concentration in the electrode 58 that imparts the electrode with thermoplasticity, self-healing, and long-term moisture retention while also increasing its low-temperature tolerance. In some instances, the concentration of the polyol compound can be about 3 wt % to about 15 wt %, about 4 wt % to about 14 wt %, about 5 wt % to about 13 wt %, about 6 wt % to about 12 wt %, about 7 wt % to about 11 wt %, or about 8 wt % to about 10 wt %.

In one example, the polyol compound is glycerol. Advantageously, glycerol can supply multiple hydroxyl groups and, thus, serve as a cross-linker for thermoplastic polymer chains (e.g., PVA) to improve the strength and toughness of hydrogels (e.g., PVA hydrogels). In some instances, the concentration of glycerol in the electrode 58 can be about 3 wt % to about 15 wt %, about 4 wt % to about 14 wt %, about 5 wt % to about 13 wt %, about 6 wt % to about 12 wt %, about 7 wt % to about 11 wt %, or about 8 wt % to about 10 wt %. In one example, the concentration of glycerol in the electrode 58 can be about 3 wt % to about 15 wt %, e.g., about 5 wt % to about 10 wt %, e.g., about 5 wt %.

In another example, the electrode 58 can comprise about 3 wt % PVA, about 5 wt % glycerol, and the carbon black can be about 50% of the weight of the PVA and glycerol.

In another aspect, the electrode 58 can absorb moisture (e.g., exudate from a wound site), without swelling, for a period of time following contact of the electrode with a source of moisture (e.g., a wound site, stratum corneum). In some instances, the period of time is about 1 hour to about 14 days, or about 24 hours to about 14 days, or about 2 days to about 14 days, or about 4 days to about 14 days, or about 6 days to about 14 days, or about 8 days to about 14 days, or about 10 days to about 14 days, or about 12 days to about 14 days. In one example, the period of time is about 5 days to about 14 days, or about 7 days to about 14 days, or about 7 days.

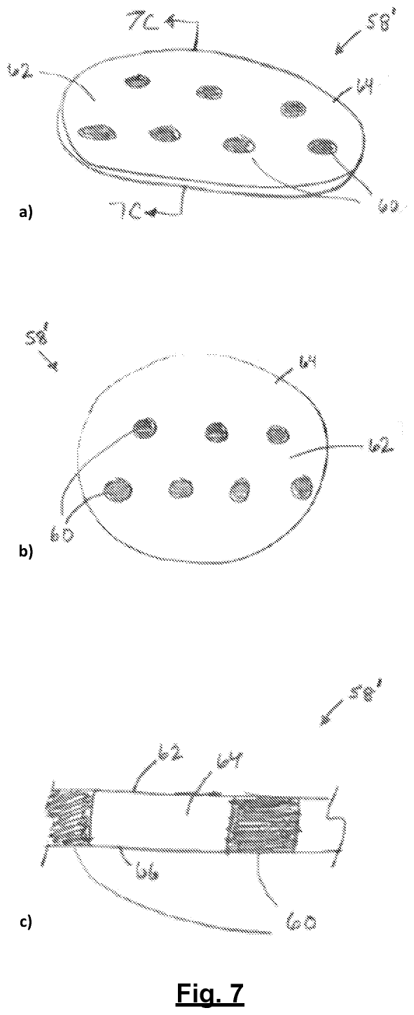

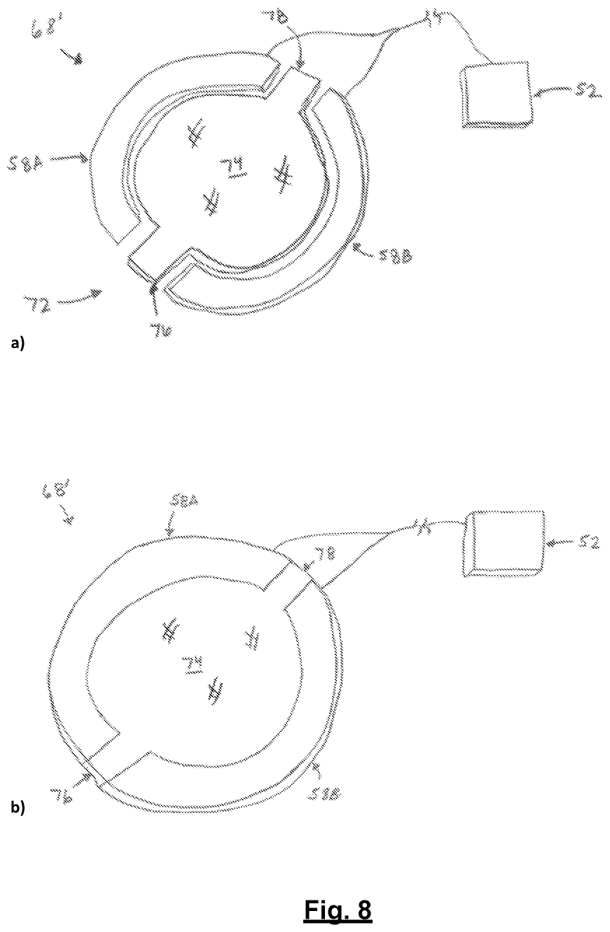

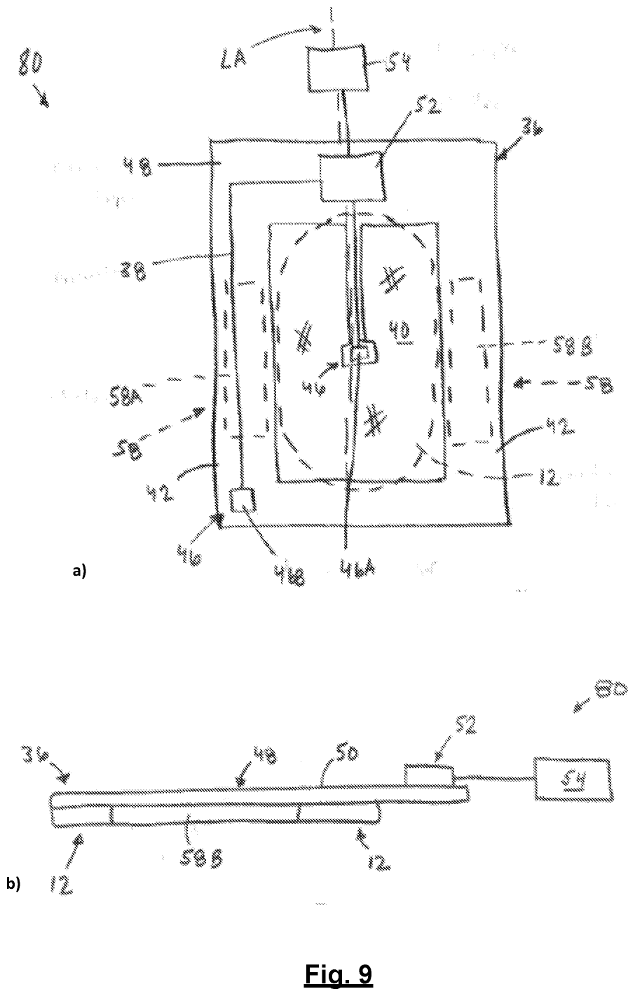

In another aspect, an electrode 58′ can be fabricated so that at least one portion or region of the electrode is transparent, and remains transparent, after absorbing moisture. As shown in FIGS. 7A-C, for example, the electrode 58′ can be fabricated to include electrically-conductive regions 60 (e.g., comprising carbon black) that are spaced apart from one another, and electrically-insulated by, transparent non-electrically-conductive regions 62 (i.e., regions that do not include, or are free of, carbon black). In some instances, each of the electrically-conductive regions 60 can extend between an opposing side 64 (FIG. 7C) and a contact side 66 of the electrode 58′. In some instances, each electrically-conductive region 60 can be in electrical communication with a separate power source (not shown). Alternatively, the electrically-conductive regions 60 can be in electrical communication with one another by, for example, a series of traces (not shown) that extend between each electrically-conductive region, at least one of which is connected to a power source. Although a checkerboard pattern of electrically-conductive regions 60 is shown in FIGS. 7A-B, it will be appreciated that any type of pattern could be created depending upon a desired stimulation pattern. The fact that the non-electrically-conductive regions 62 remain transparent after absorbing moisture is advantageous as it permits observation of a wound site, contemporaneous with application of electrical stimulation, so that wound healing can be observed and the frequency of bandage changes is reduced.

It will be appreciated that the dimensions and shape of an electrode 58 can be tailored for any given application. Although the dimensions of the electrode 58 can be tailored for any given application, it is contemplated that rectangular shapes can be preferable due to the conductive pathways created by the carbon black particles. In some instances, for example, the electrode 58 can have a regular shape (e.g., circle, rectangle, square) or an irregular shape. In some aspects, the electrode 58 has a thickness T (FIG. 6B), which can be defined as the distance between the contact side 66 and the opposing side 64, where the contact side is configured to contact the stratum corneum (or other tissue) of a subject. The thickness T of the electrode 58 can be selected to provide the electrode with flexibility sufficient to conform to the irregular topography of the stratum corneum. In one example, the electrode 58 can have a thickness T of between about 10 microns and about 10 mm, or between 50 microns and about 200 microns, or about 100 microns.

In another aspect, electrodes 58 of the present disclosure can have a resistance at or below 1 kΩ and a resistivity of less than 1 Ω-m, thereby achieving parameters suitable for bioelectronics. For example, in some instances, the resistivity can be about 0.1 Ω-m to about 20 Ω-m, about 0.5 Ω-m to about 15 Ω-m, about 1 Ω-m to about 10 Ω-m, or about 2 Ω-m to about 8 Ω-m. In a further example, the resistivity can be about 0.1 Ω-m to about 0.5 Ω-m, e.g., about 0.1 Ω-m (e.g., about 0.215 Ω-m). In other instances, the electrodes 58 of the present disclosure can have a resistance between 50Ω and about 1 kΩ, or about 100Ω to about 900Ω, or about 200Ω to about 800Ω, or about 300Ω to about 700Ω, or about 400Ω to about 700Ω, or about 500Ω to about 600Ω. In one example, the resistance can be between about 0.3 kΩ and about 700Ω, e.g., about 859Ω. It should be understood that other sensors comprising carbon black, PVA, and glycerol have been produced for applications such as strain gauges. However, such other sensors have resistances of at least 20 kΩ, rendering them inoperable or unusable for bioelectronics (e.g., as a bioelectronics electrode component of a bioelectronics sensor).

Over long durations electrodes 58 of the present application can exhibit little-to-no fatigue. That is, performance (e.g., long-term electrical stability) of the electrodes 58 does not change substantially over time. In some instance, performance (e.g., long-term electrical stability) of an electrode 58 of the present application can be maintained, or substantially maintained, over a period of about 1 day to about 30 days, about 2 days to about 28 days, about 3 days to about 26 days, about 4 days to about 24 days, about 5 days to about 22 days, about 6 days to about 20 days, about 7 days to about 18 days, about 8 days to about 16 days, about 8 days to about 14 days, about 9 days to about 12 days, or about 10 days to about 11 days. In one example, performance (e.g., long-term electrical stability) of an electrode 58 of the present application can be maintained, or substantially maintained, over a period of 1 day to about 25 days. In another example, performance (e.g., long-term electrical stability) of an electrode 58 of the present application can be maintained, or substantially maintained, over a period of less than about 25 days (e.g., 25 days). In yet another example, performance (e.g., long-term electrical stability) of an electrode 58 of the present application can be maintained, or substantially maintained, for about 25 days (e.g., 25 days).

The flexibility of the electrode 58 can be quantified in terms of the material's Young's modulus. The Young's modulus of the electrode 58 can increase with increasing carbon black concentration. In one example, an electrode 58 having a weight of carbon black that is about 50% of the weight of thermoplastic material (e.g., PVA) and polyol compound (e.g., glycerol) can have a Young's Modulus of between about 10 MPa and 15 MPa, e.g., about 12 MPa (e.g., 11.98 MPa). Thus, the electrodes 58 of the present disclosure can be distinguishable from commercially available electrodes such as, for example RED DOT Ag/AgCl electrodes provided by 3M, which are not flexible or stretchable. The commercially available electrodes are encapsulated with a conductive gel overlaid with an adhesive on a foam bedding to adhere to the stratum corneum. The commercially available electrodes cause irritation in at least 10% of patients, have limited shelf stability and disposability, and demonstrate signal degradation over time. Thus, use of the commercially available electrodes is limited outside of clinical environments. The electrodes 58 as disclosed herein have desirable properties that can allow the electrodes to adhere to the irregular topography of the stratum corneum without causing irritation.

In another aspect, electrodes 58 of the present disclosure can have an electrical conductance of between about 10 S/m and about 200 S/m, or about 20 S/m to about 180 S/m, or about 30 S/m to about 160 S/m, or about 40 S/m to about 140 S/m, or about 50 S/m to about 120 S/m, or about 60 S/m to about 100 S/m, or about 70 S/m to about 80 S/m. In one example, electrodes 58 of the present application can have an electrical conductance of about 10 S/m to about 20 S/m, or about 12 S/m to about 18 S/m, or about 14 S/m to about 16 S/m. In another example, electrodes 58 of the present application can have an electrical conductance of about 12 S/m to about 14 S/m, e.g., about 13 S/m.

Optionally, in some instances, an adhesive patch (not shown) can be used to maintain such an electrode 58 in engagement with the skin of a subject.

In further instances, a control module 52 can be in electrical communication with the electrode 58 via wires or other electrical leads; however, it will be appreciated that the control module can alternatively be in wireless communication with the electrode. In some instances, the control module 52 can comprise a computing device (e.g., a microcontroller) (as described above) and, optionally, a power source (e.g., a battery). It will be appreciated that, where the power source is not part of the control module 52, the power source may comprise a separate component of the electrode assembly 68 and, as such, also be in electrical communication with the electrode 58. The control module 52 can be configured to receive signals from the electrode 58 and process the signals (e.g., convert analog signals to digital and store the signals with correlated time values). Additionally, or alternatively, it is contemplated that the electrode 58 and/or the control module 52 can be in electrical communication (e.g., wirelessly communicate) with a remote device 54, such as, for example, a tablet, a smartphone, or a computer.

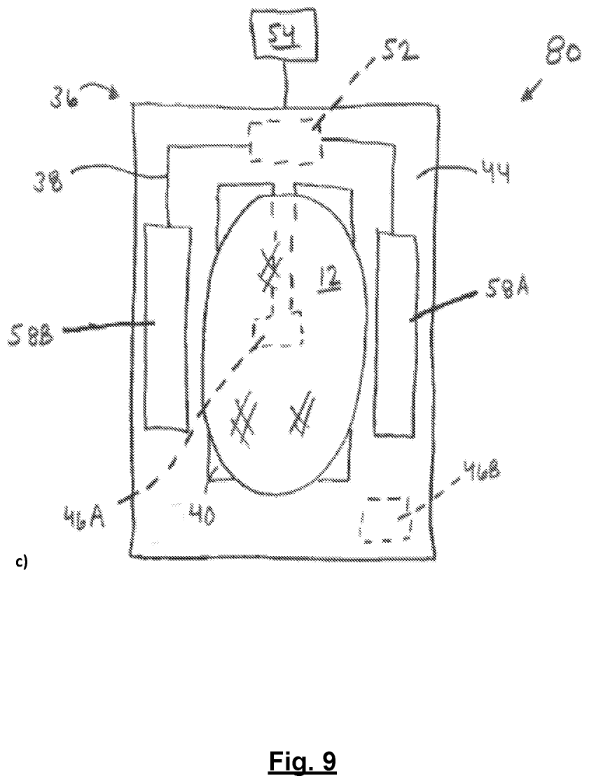

Another example of an electrode 58″ according to the present disclosure is illustrated in FIGS. 8A-B. In this example, a composite electrode 58″ can comprise a central moisture absorbing layer 72 seated between first and second electrodes 58A and 58B. In one aspect, the central moisture absorbing layer can be formulated similar or identical to the moisture absorbing layer 12 described in Section (I) above. The central moisture absorbing layer 72 can include a circular central region 74 and opposing first and second tabs 76 and 78 extending from the central region. In another aspect, each of the first and second electrodes 58A and 58B can be formulated as the electrode 58 described in this Section (Section IV). Each of the first and second electrodes 58A and 58B can have a semi-circular or half-mood shape and be connected to the central moisture absorbing layer 72 on opposite side edges thereof. In some instances, the central moisture absorbing layer 72 and the first and second electrodes 58A and 58B can be joined or connected to one another using a suitable adhesive. In other instances, an additive manufacturing technique (e.g., 3D printing) or an extrusion technique can be used to form the composite electrode 58″ as a single, continuous structure without separate components that need to be adhered to one another.

In the configuration shown in FIGS. 8A-B, the central moisture absorbing layer 72 serves as an insulating barrier between the first and second electrodes 58A and 58B. In a further aspect, the composite electrode 58″ can include a control module 52 and/or remote device 54 that is/are in electrical communication with the first and second electrodes 58A and 58B.

In an exemplary method, the nanocomposite electrode 58″ can be used as part of a method (e.g., a closed-loop method) for healing a wound site of a subject. In such method, the nanocomposite electrode 58″ can be over a wound site. Next, a series of electrical stimulations can be applied to the wound site, by the first and second electrodes 58A and 58B, until the wound site is healed. In some instances, the series of electrical stimulations can be based on a received impedance measurement from between the first and second electrodes 58A and 58B. In some aspects, the first and second electrodes 58A and 58B can be in electrical communication with a control module 52 that is operative to: receive the impedance measurement from between the first electrode and the second electrode; and, optionally, transmit, to a remote device 54, a signal corresponding to the impedance measurement.

In some instances, the duration of ES (as well as other stimulation parameters, such as pulse-width and amplitude) can be modulated in response to detected or sensed changes in impedance from the first electrode 58A and/or the second electrode 58B. In other instances, the direction of ES (current) applied to the wound site can be determined by the control module 52. Thus, delivery of ES can be driven directionally by which electrode 58 (or electrodes) respond(s) to impedance changes. Given that wound re-epithelialization does not occur uniformly, such an approach advantageously enables site-specific delivery of the electroceutical therapy to a region (or regions) of interest.

Electrodes 58 of the present disclosure can be used in bioelectronic applications for, for example, monitoring and treating human health and performance. The disclosed electrodes 58 can be integrated with a flexible electroceutical system for health maintenance and monitoring. Other potential applications include but are not limited to:
Integration with electrotherapeutic system for prevention of surgical site infections
Integration with electrotherapeutic system for treatment of acute wounds
Integration with electrotherapeutic system for treatment of chronic wounds
Integration with electrotherapeutic system for treatment of wound infection
Integration with electrotherapeutic system for pain management
Surface functional electrical stimulation of muscles
Remote patient activity monitoring
Ambulatory activity monitoring
Integration with ECG, EEG, EMG technology
Fluid flow monitoring, e.g., hydrocephalus
Iontophoresis/Reverse Iontophoresis
Drug Delivery
Biomarker extraction from eccrine or apocrine sweat
Integration with stretchable electronics

In some exemplary, non-limiting aspects, the electrodes 58 as disclosed herein can be used in devices for remotely monitoring and treating wound infections (e.g., using electrotherapy). The devices can be used in combination with various processing, monitoring, and/or treatment components to provide a system. Optionally, the disclosed device can be communicatively coupled to a remote device, such as a computer, a tablet, a smartphone, and the like. Optionally, such remote devices can include processing circuitry that is configured to execute application software that remotely controls and monitors operation of the device. Examples of such devices are described in Section (V) below.

### V

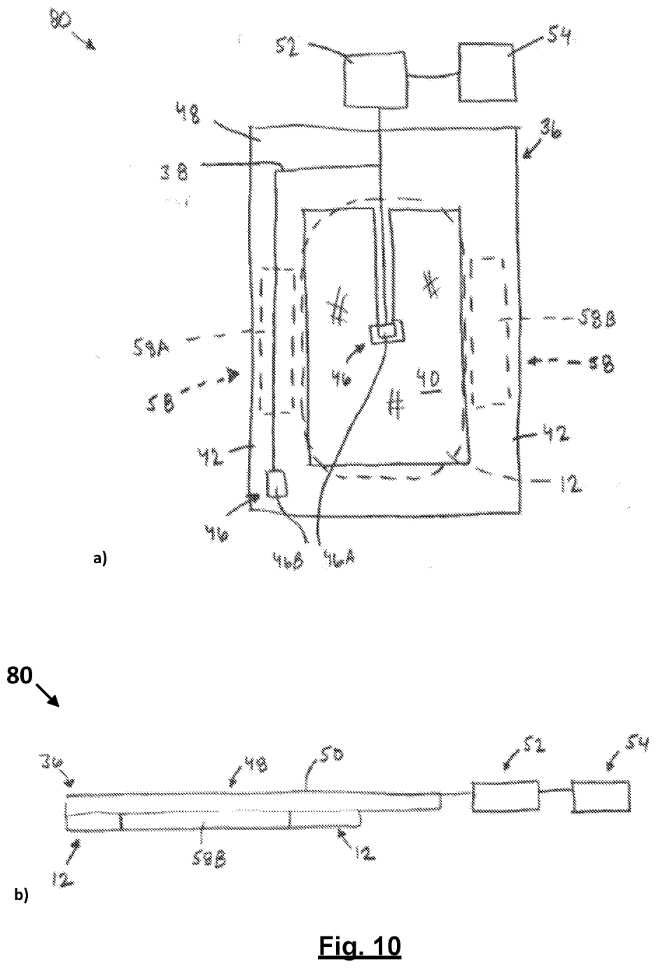

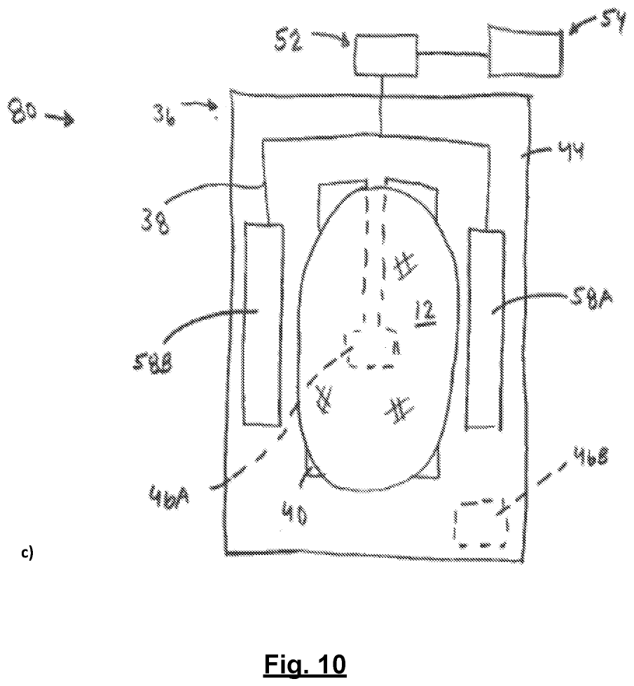

Another aspect of the present disclosure, illustrated in FIGS. 9A-C and FIGS. 10A-C, can include devices useful for, for example, monitoring and/or healing a wound site. As discussed below, such devices are advantageously beneficial in applications that require wicking of moisture (e.g., bodily fluids) by virtue of the moisture absorbing layer concurrently with delivery of surface stimulation (as further disclosed herein) by virtue of electrodes disclosed herein.

Referring to FIGS. 9A-C, a device 80 according to one aspect of the present disclosure can have a layered construction. A packaged control module 52 and/or remote device 54 can be provided with the device 80. In exemplary non-limiting aspects, the device 80 can comprise: (1) electrodes 58 and sensors (e.g., temperature sensors 48 and/or impedance sensors); (2) an occlusive layer 36 having circuitry 38 therein for coupling the control module 52 to temperature sensors and electrodes; (3) a moisture absorbing layer 12 to manage wound exudate; and (4) an adhesive patch to ensure contact of the device with the wound and periwound area.

In some aspects, the moisture absorbing layer 12 can be embodied as the moisture absorbing layer disclosed in Section (I).

In further aspects, the electrodes 58 can be embodied as the flexible electrodes disclosed in Section (IV). In further optional aspects, electrodes can be constructed from conductive fabric. Optionally, in these aspects, it is contemplated that a suitable conductive fabric for this application can have low impedance, can maintain a stable voltage over the length of the electrode, will not heat when sustained current is applied, and can remain chemically stable when sterilized and when exposed to the wound environment. Examples of suitable conductive fabrics include SHIELDEX TECHNIK-TEX P130+B and SHIELDEX TECHNIK-TEX P130+B conductive fabrics sold by V Technical Textiles, Inc. of Palmyra, NY.

In still further aspects, the electrodes 58 can be attached to the control module 52 using conductive thread, which can serve as a conductive via for vertical interconnects. Examples of a suitable conductive thread can include 235/43 DTEX HC+B conductive thread sold by V Technical Textiles, Inc. of Palmyra, NY. An appropriate adhesive can also be used to mechanically and electrically secure the electrodes 58. Optionally, the adhesive can be a conductive adhesive, such as a conductive epoxy adhesive. Suitable adhesives include MG Products 8331 silver conductive epoxy adhesive.

In some embodiments, the device 80 can comprise a top occlusive layer 36 and the moisture absorbing layer 12. In various aspects, the top occlusive layer 36 can be reusable, and the moisture absorbing layer 12 can be disposable. Accordingly, in some aspects, the moisture absorbing layer 12 can be positioned over the wound site, and the top occlusive layer 36 can be positioned over the moisture absorbing layer.

The top occlusive layer 36 can optionally comprise a flexible, transparent window 40 and a flexible adhesion portion 40 that extends around the circumference of the transparent window and is configured to adhere to the skin of the subject to enclose the moisture absorbing layer 12 between the occlusive layer and the subject (as described above).

A medical grade pressure sensitive adhesive coating can be disposed on a wound side 16 of at least a portion of moisture absorbing layer 12 for adhering the device 80 to a subject.

A plurality of electrodes 58 (optionally, a first electrode 58A and a second electrode 58B) can be disposed on the wound side 44 of the occlusive layer 36 and/or the moisture absorbing layer 12. Optionally, an adhesive can be disposed on the wound side 44 to help it adhere to the skin of a subject. Optionally, the moisture absorbing layer 12 can define holes (not shown) therethrough, and the electrodes 58 can be positioned within the holes and attached to the underside of the occlusive layer 36. In further instances, the electrodes 58 can attach to the wound side 16 of the moisture absorbing layer 12. In still further aspects, the electrodes 58 can be integral to the moisture absorbing layer 12 and positioned on the wound side 16 of the moisture absorbing layer. In use, it is contemplated that the electrodes 58 can be configured to provide electrical stimulation as further disclosed herein.

A plurality of temperature sensors 46 (optionally, a first temperature sensor 46A and a second temperature sensor 46B) can be configured to detect the temperature at the skin/wound of the subject. The plurality of temperature sensors 46 can be coupled to (and in electrical communication with) the flexible circuitry 38 as described in Section (II). Additionally, the plurality of temperature sensors 46 can be configured about the device 80 as described in Section (II).

The electrodes 58 can be periwound electrodes. That is, in use, the electrodes 58 can be positioned on the skin surrounding the wound site and, thus, be spaced (e.g., slightly spaced) from the wound bed (i.e., the area of the wound).

The first temperature sensor 46A can be positioned between the electrodes 58, and the second temperature sensor 46B can be positioned outside of the electrodes. Thus, the first temperature sensor 46A can be positioned over or within the wound bed, while the second temperature sensor 46B can be positioned away from (i.e., depending on the orientation of the wound and the device 80, laterally or vertically spaced from) the wound bed. For example, when the wound bed is oriented horizontally, it is contemplated that the second temperature sensor 46B can be sufficiently horizontally spaced from the wound bed so that the temperature measured by the second temperature sensor reflects ambient/systemic temperature information (rather than the temperature at or within the wound). As shown in FIG. 9A, the device 80 can have a longitudinal axis LA. Optionally, the first and second temperature sensors 46A and 46B and the first and second electrodes 58A and 58B can be aligned along the longitudinal axis LA. In some instances, along the longitudinal axis LA, the first temperature sensor 46A can be disposed between the first and second electrodes 58A and 58B, and the second temperature sensor 46B can be disposed outside of the space between the first and second electrodes. Thus, when the electrodes 58 are positioned on opposite sides of the wound bed, the first temperature sensor 46A can be positioned in the wound bed, and the second temperature sensor 46B can be positioned outside the wound bed (on the opposite side of the second electrode 58B from the first temperature sensor 46A).

In some instances, the first and second temperature sensors 46A and 46B can be spaced from each other by at least 1.5 centimeters, at least two centimeters, or at least four centimeters (e.g., from about two centimeters to about three centimeters, from about three centimeters to about four centimeters, from about four centimeters to about five centimeters, from about five centimeters to about six centimeters, from about six centimeters to about seven centimeters, from about seven centimeters to about eight centimeters, from about eight centimeters to about nine centimeters, from about nine centimeters to about ten centimeters, from about ten centimeters to about twelve centimeters, or more.

In some instances, the first and second electrodes 58A and 58B can be spaced from each other by at least two centimeters (e.g., from about two centimeters to about three centimeters, from about three centimeters to about four centimeters, from about four centimeters to about five centimeters, from about five centimeters to about six centimeters, from about six centimeters to about seven centimeters, from about seven centimeters to about eight centimeters, from about eight centimeters to about nine centimeters, from about nine centimeters to about ten centimeters, from about ten centimeters to about twelve centimeters, from about twelve centimeters to about fifteen centimeters, from about fifteen centimeters to about twenty centimeters, or from about twenty centimeters to about twenty-five centimeters or more. Optionally, it is contemplated that the spacing between the first and second electrodes 58A and 58B can be greater than the spacing between the first and second temperature sensors 46A and 46B. Alternatively, it is contemplated that the spacing between the first and second electrodes 58A and 58B can be equal or substantially equal to the spacing between the first and second temperature sensors 46A and 46B. In still a further alternative, it is contemplated that the spacing between the first and second electrodes 58A and 58B can be less than the spacing between the first and second temperature sensors 46A and 46B.

As shown schematically in FIGS. 9A-C, a control module 52 can be in electrical communication with the electrodes 58 and the temperature sensors 46. The control module 52 can electrically couple to the electrodes 58 and the temperature sensors 46 by flexible printed circuitry 38. The flexible printed circuitry 38 can define conductors (not shown) that extend from the control module 52, along the top occlusive layer 36. Optionally, the conductors can comprise conductive traces (e.g., copper traces). However, it is contemplated that any conventional conductive material can be used. Optionally, the flexible printed circuitry 38 can be integrally formed with the top occlusive layer 36. In some optional aspects, the flexible printed circuitry 38 can comprise the temperature sensors 46. For example, the temperature sensors 46 can be integrally formed with the flexible printed circuitry 38 or soldered thereto. The flexible printed circuitry 38 can define electrical contacts (not shown) that can form electrical communication with the electrodes 58. Optionally, for example, a respective electrical conductor can extend from the electrodes 58 to an outer side 18 of the moisture absorbing layer 12 where it engages the respective contact. The control module 52 can include a power source, such as a battery. In this way, the device 80 can be portable and can omit cables or wires extending therefrom. Alternatively, it is contemplated that the control module 52 can be electrically coupled to an external power source (for example, using a cord or cable).

As shown in FIGS. 9A-C, the control module 52 can be located on an upper side 48 of the occlusive layer 36 and be in electrical communication with a remote device 54, which is physically spaced from, and not located on, the occlusive layer. Optionally, in an alternative configuration, the control module 52 can be physically spaced apart from the occlusive layer 36 (e.g., not physically located on the occlusive layer) but still remain in electrical communication with the flexible circuitry 38 of the device 80 (FIGS. 10A-C).

In one aspect, the flexible printed circuitry 38 of the device 80 can comprise a copper (Cu)-clad flex-electronics polyimide (or other suitable biocompatible) sheet.

Photolithographic patterning can be used to fabricate Cu contact pads for coupling to the electrodes 58 on the wound (bottom) side 44 to the flexible printed circuitry 38 as well as interconnect traces for communicating electrical current to and from the electrodes and temperature sensors 46. The electrodes 58 can optionally be multi-layered, multi-material electrodes.

The moisture absorbing layer 12 can be placed over the wound with the electrodes 58 and temperature sensors 46 positioned as described herein. The occlusive layer 36 can be positioned over the moisture absorbing layer 12 so that the control module 52 is in communication with the electrodes 58 and temperature sensors 46. After use, the moisture absorbing layer 12 can be removed from the wound site and from the occlusive layer 36 and disposed of. In some aspects, the occlusive layer 36 can be sterilized for reuse. In some optional aspects, the control module 52 can be removed prior to sterilization of the occlusive layer 36.

### VI

Another aspect of the present disclosure can include a method for healing a wound site of a subject. In one example, the wound site is an acute wound. In another example, the wound site is a chronic wound. One step of the method can include applying a device 80 (as described in Section (V)) over the wound site. Next, a series of electrical stimulations can be applied by the electrodes 58 of the device 80 to the wound site until the wound site is healed. The series of electrical stimulations can be based on a received temperature measurement and/or a received impedance measurement from between the first and second electrodes 58A and 58B and from between each temperature sensor 46A and 46B of the plurality of temperature sensors 46, respectively. Further description of electrical stimulation by a device 80 of the present disclosure is provided below.

In exemplary aspects, the temperature sensors 46 disclosed herein can measure a change in resistance that can be converted to a corresponding temperature change using conventional methods (for example, using TCR parameters). In use, at least one temperature sensor 46A can be located over the wound bed (i.e., the area of the wound), and at least one other temperature sensor 46B can be located over intact periwound skin. The temperature sensor 46B located over the periwound skin (i.e., spaced away from the wound area) can provide ambient/systemic temperature that can provide insight to the local wound microenvironment. The temperature sensors 46 can be intentionally made with minimum sufficient surface area in contact with the wound or skin of the subject so as to minimize their impact on the electrical stimulation (ES) performance of the device 80. In exemplary aspects, it is contemplated that the actual “contact” surface area between each temperature sensor 46 and the subject can range from about one square millimeter to about 200 square millimeters, from about 1.25 square millimeters to about 150 square millimeters, from about 1.5 square millimeters to about 100 square millimeters, from about 1.75 square millimeters to about 25 square millimeters, or from about two square millimeters to about five square millimeters. Thermal noise can be corrected by subtracting the periwound temperature measurement from the measurement from the sensor 46A located over the wound bed. In use, the sensors 46 can exhibit a linear response within the clinically relevant range of about 35° C. to about 40° C. (about 95° F. to about 104° F.). The temperature sensors 46 can optionally provide accurate temperature measurements to within about 0.1° C. within the clinically relevant range.

It is contemplated that changes in wound impedance (i.e., the impedance across the wound) over time can be an indicator of progress of wound closure and healing (as described above). Thus, measurement of the wound impedance can enable monitoring of both progress of the wound as well as excess moisture accumulation. The impedance between the electrodes 58 can be measured in intervals between deliveries of therapeutic ES. In this way, a clinician can remotely monitor the status of the wound in real time without disturbing the wound environment.

In another aspect, the control module 52 can control the current and/or voltage to the electrodes 58 for providing ES. The control module 52 can be operative to control an electrical current between at least two electrodes 58A and 58B of the plurality of electrodes 58 to provide a series of electrical stimulations to a wound site. Optionally, the series of electrical stimulations can be varied in accordance with a desired pattern or scheme. Further, the control module 52 can measure impedance between electrodes 58 using conventional methods. Optionally, it is contemplated that the control module 52 can comprise or be in communication with an impedance meter as is known in the art. In use, the impedance meter, through the control module 52, can apply an AC voltage source across the wound site. The impedance meter can receive signals from the electrodes 58 that are indicative of the voltage across and the current through the wound site. Using conventional techniques, the impedance meter can then determine the magnitude of the impedance based upon the ratio between the measured voltage and the measured current. In some instances, the control module 52 can comprise or be in communication with a display for displaying various information, including temperature measurements and impedance measurements.

In some aspects, the control module 52 can be communicatively coupled (i.e., communicate using wired or wireless connection) to a remote device 54. In some instances, the remote device 54 can be a remote monitor. In further instances, the remote device 54 can perform certain control and/or processing functions. For example, the control module 52 can receive signals from the temperature sensors 46 (e.g., signals indicative of resistance measurements by the sensors as disclosed herein). In some instances, the control module 52 can process said signals for conversion to a temperature measurement (e.g., using TCR parameters). In these instances, it is contemplated that the control module 52 can comprise at least one processor and a memory that stores instructions that, when executed by the at least one processor, determine the temperature measurement based on the received signals. In further instances, the remote device 54 can receive and process said signals for conversion to a temperature measurement. Similarly, the remote device 54 can display various information, including temperature measurements and impedance measurements. Further, the remote device 54 can provide an interface through which a clinician can control the device 80 (e.g., begin or end the electrical stimulation as well as change the properties of the ES).

Optionally, in exemplary aspects, the remote device 54 can be provided as a remote computing device, such as, for example and without limitation, a smartphone, a tablet, a laptop computer, or a desktop computer. In these aspects, it is further contemplated that the remote device 54 can comprise at least one processor and a memory in communication with the processor. The memory can store instructions that, when executed by the processor, determine information concerning the wound of the patient, including the temperature measurement, impedance measurements, change in temperature, and change in impedance. The memory can further store additional information related to device use as well as battery status. The control module 52 can be configured for internet connectivity, optionally, through the remote device 54. In this way, data from the control module 52 can be uploaded to a remote server. Thus, a clinician can remotely monitor the status of the wound site. Further, in some optional aspects, the control module 52 can receive instructions from the internet or closed network, for example, to modify the electrotherapy.

In exemplary aspects, it is contemplated that the control module 52 and the remote device 54 (when provided) can comprise respective user interfaces (e.g., keyboards, touchscreens, dials, and the like) that allow for direct communication between a clinician and the control module and remote module. In use, it is contemplated that the control module 52 and/or the remote device 54 can be configured to control and adjust the duration, intensity/voltage, and/or frequency of the ES that is delivered through the electrodes 58 as disclosed herein.

Optionally, the control module 52 and/or the remote device 54 can be configured to determine an ischemic status of the wound based on a temperature difference between the first temperature sensor 46A and the second temperature sensor 46B. Optionally, the control module 52 and/or the remote device 54 can be configured to determine a healing status based on changes in impedance between the first and second electrodes 58A and 58B

It will be appreciated that other hardware and software components of the control module 52 and the remote device 54 can be included, such as those hardware and software components described in Section (II).

### Exemplary Aspects

In view of the described compositions, devices, systems, and methods and variations thereof, herein below are certain more particularly described aspects of the present disclosure. These particularly recited aspects should not, however, be interpreted to have any limiting effect on any different claims containing different or more general teachings described herein, or that the “particular” aspects are somehow limited in some way other than the inherent meanings of the language literally used therein.

Aspect 1: A wound dressing for application against a wound site of a subject, the wound dressing comprising: a transparent, moisture absorbing layer having a wound side and an opposed outer side; and an adhesive layer that is connected to at least a portion of the wound side of the moisture absorbing layer, wherein the adhesive layer facilitates attachment of the wound dressing to a non-wounded perimeter of the wound site; wherein the moisture absorbing layer absorbs moisture from the wound site, without swelling, to promote healing of the wound site.

Aspect 2: The wound dressing of aspect 1, wherein the moisture absorbing layer comprises a thermoplastic material and a polyol compound.

Aspect 3: The wound dressing of any one of aspects 1-2, wherein the thermoplastic material is poly(vinyl alcohol) (PVA).

Aspect 4: The wound dressing of any one of aspects 1-3, wherein the concentration of the PVA in the moisture absorbing layer is about 1 to 5 wt %.

Aspect 5: The wound dressing of any one of aspects 1-4, wherein the concentration of the PVA in the moisture absorbing layer is about 3 wt %.

Aspect 6: The wound dressing of any one of aspects 1-5, wherein the polyol compound is glycerol.

Aspect 7: The wound dressing of aspect 6, wherein the concentration of the glycerol is about 3 to 15 wt %.

Aspect 8: The wound dressing of any one of aspects 6-7, wherein the concentration of the glycerol is about 5 to 10 wt %.

Aspect 9: The wound dressing of any one of aspects 6-8, wherein the concentration of the glycerol is about 5 wt %.

Aspect 10: The wound dressing of any one of aspects 1-9, wherein the moisture absorbing layer remains transparent after absorbing liquid from the wound site.

Aspect 11: The wound dressing of any one of aspects 1-10, wherein the moisture absorbing layer absorbs liquid from the wound site, without swelling, for a period of about 1 hour to about 14 days following contact of the wound dressing with the wound site.

Aspect 12: The wound dressing of any one of aspects 1-11, wherein the moisture absorbing layer includes one or more bioactive agents for delivery into tissue comprising the wound site or a surrounding non-wound site.

Aspect 13: The wound dressing of any one of aspects 1-12, wherein the moisture absorbing layer is free of any exogenous conductive elements.

Aspect 14: The wound dressing of any one of aspects 1-12, being fabricated by additive manufacturing, e.g., three-dimensional (3D) printing.

Aspect 15: A device comprising: a transparent, moisture absorbing layer having a wound side and an opposed outer side, wherein the moisture absorbing layer absorbs moisture, without swelling, from a wound site; an occlusive layer positioned against at least a portion of the outer side of the moisture absorbing layer, wherein the occlusive layer comprises flexible circuitry that defines a plurality of electrical contacts; and a plurality of temperature sensors coupled to the flexible circuitry; wherein each temperature sensor of the plurality of temperature sensors is in electrical communication with a respective contact of the plurality of contacts of the flexible circuitry.

Aspect 16: The device of aspect 15, further comprising a control module in electrical communication with the plurality of temperature sensors, wherein the control module is operative to receive and/or store a signal from each temperature sensor of the plurality of temperature sensors.

Aspect 17: The device of any one of aspects 15-16, wherein the plurality of temperature sensors comprises at least a first temperature sensor and a second temperature sensor, wherein the control module is further operative to: receive a temperature measurement from between the first temperature sensor and the second temperature sensor; and transmit, to a remote device, a signal corresponding to the temperature measurement.

Aspect 18: The device of any one of aspects 15-17, further comprising an adhesive patch connected to the moisture absorbing layer and/or the occlusive layer.

Aspect 19: The device of any one of aspects 15-18, further comprising a plurality of electrodes disposed over the wound side of the moisture absorbing layer, or disposed over a wound side of the occlusive layer, wherein each electrode of the plurality of electrodes is an elastomeric composite and is in electrical communication with a respective contact of the plurality of contacts of the flexible circuitry.

Aspect 20: The device of any one of aspects 15-19, being fabricated by additive manufacturing, e.g., 3D printing.

Aspect 21: A method for monitoring healing of a wound site, the method comprising the steps of: positioning the device of any one of aspects 15-20 on a subject having a wound site so that a first temperature sensor of the plurality of temperature sensors is positioned within or over the wound site and a second temperature sensor of the plurality of temperature sensors is positioned at a location spaced apart from the wound site; and determining, by a processing device, a status of the wound based on a temperature difference between the first temperature sensor and the second temperature sensor.

Aspect 22: The method of aspect 21, wherein the status is an infection status of the wound site.

Aspect 23: The method of aspect 22, wherein the status is an ischemic status of the wound site.

Aspect 24: A method for healing a wound site of a subject, the method comprising: applying the wound dressing of any one of aspects 1-14, or the device of any one of aspects 15-20, over the wound site; optionally applying, by the electrodes, a series of electrical stimulations to the wound site; and leaving the wound dressing or the device over the wound site for a period of time until the wound site is healed.

Aspect 25: The method of aspect 24, wherein the wound site is a chronic wound.

Aspect 26: The method of aspect 24, wherein the wound site is an acute wound.

Aspect 27: An electrode comprising: carbon black; a thermoplastic material; and a polyol compound; wherein the carbon black is provided at a weight of between about 10% and 70% of a weight of the thermoplastic material and the polyol compound; wherein the electrode is formulated to absorb moisture without swelling.

Aspect 28: The electrode of aspect 27, wherein the weight of the carbon black is between about 35 and 60% of the weight of the thermoplastic material and the polyol compound.

Aspect 29: The electrode of any one of aspects 27-28, wherein the weight of the carbon black is about 50% of the weight of the thermoplastic material and the polyol compound.

Aspect 30: The electrode of any one of aspects 27-29, wherein the thermoplastic material is PVA.

Aspect 31: The electrode of aspect 30, wherein the concentration of the PVA in the electrode is about 1 to 5 wt %.

Aspect 32: The electrode of any one of aspects 30-31, wherein the concentration of the PVA in the electrode is about 3 wt %.

Aspect 33: The electrode of aspect 27, wherein the polyol compound is glycerol.

Aspect 34: The electrode of aspect 33, wherein the concentration of the glycerol is about 3 to 15 wt %.

Aspect 35: The electrode of any one of aspects 33-34, wherein the concentration of the glycerol is about 5 to 10 wt %.

Aspect 36: The electrode of any one of aspects 33-35, wherein the concentration of the glycerol is about 5 wt %.

Aspect 37: The electrode of any one aspects 27-36, wherein at least one region of the electrode is a transparent, non-electrically-conductive region and at least one other region of the electrode is a non-transparent, electrically-conductive region; wherein the at least one other region is surrounded, and electrically insulated by, the at least one transparent, non-electrically-conductive region.

Aspect 38: The electrode of aspect 37, wherein the at least one portion of the electrode that is transparent remains transparent after absorbing moisture.

Aspect 39: The electrode of any one of aspects 27-38, wherein the electrode absorbs moisture, without swelling, for a period of about 1 hour to about 14 days.

Aspect 40: The electrode of any one of aspects 27-39, wherein the electrode has a resistivity of less than about 1 Ω-m.

Aspect 41: The electrode of aspect 40, wherein the electrode has a resistivity of about 0.2 Ω-m.

Aspect 42: The electrode of any one of aspects 27-39, wherein the electrode is physically free from contact with a conductive gel.

Aspect 43: The electrode of aspect 42, wherein the conductive gel is a hydrogel.

Aspect 43: The electrode of any one of aspects 27-43, being fabricated by additive manufacturing, e.g., 3D printing.

Aspect 44: The electrode of any one of aspects 27-43, being configured as a surface electrode for transcutaneous electrical stimulation of a nerve (e.g., a peripheral nerve) and/or muscle.

Aspect 45: The electrode of any one of aspects 27-44, having an electrical conductance of about 10 S/m to about 20 S/m, or about 12 S/m to about 18 S/m, or about 14 S/m to about 16 S/m, or about 13 S/m.

Aspect 46: The electrode of any one of aspects 27-45, being operative as a temperature sensor.

Aspect 47: The electrode of any one of aspects 27-45, being operative as an impedance sensor.

Aspect 48: A device comprising: a moisture absorbing layer having a wound side and an opposed outer side; a plurality of electrodes disposed over the wound side of the moisture absorbing layer, wherein each electrode of the plurality of electrodes is an electrode of any one of aspects 27-47; an occlusive layer positioned against the outer side of the moisture absorbing layer, wherein the occlusive layer comprises flexible circuitry that defines a plurality of electrical contacts; and a plurality of temperature sensors coupled to the flexible circuitry; wherein each electrode of the plurality of electrodes is in electrical communication with a respective contact of the plurality of contacts of the flexible circuitry.

Aspect 49: The device of aspect 48, wherein the moisture absorbing layer is a moisture absorbing layer of any one of aspects 1-14.

Aspect 50: The device of any one of aspects 48-49, further comprising: a control module in electrical communication with the plurality of electrodes and the plurality of temperature sensors, wherein the control module is operative to: control an electrical current between at least two electrodes of the plurality of electrodes to provide a series of electrical stimulations to a wound site; and receive a signal from each temperature sensor of the plurality of temperature sensors.

Aspect 51: The device of any one of aspects 48-50, wherein the plurality of electrodes comprises at least a first electrode and a second electrode, wherein the control module is further operative to: receive an impedance measurement from between the first electrode and the second electrode; and transmit, to a remote device, a signal corresponding to the impedance measurement.

Aspect 52: The device of any one of aspects 48-51, further comprising an adhesive patch connected to the moisture absorbing layer and/or the occlusive layer.

Aspect 53: The device of any one of aspects 48-52, being fabricated by additive manufacturing, e.g., 3D printing.

Aspect 54: A method for healing a wound site of a subject, the method comprising: applying the electrode of any one of aspects 27-47, or the device of any one of aspects 48-52 over the wound site; and applying, by the electrodes, a series of electrical stimulations to the wound site until the wound site is healed, the series of electrical stimulations being based on a received temperature measurement and/or a received impedance measurement from between the first and second electrodes and from between each temperature sensor of the plurality of temperature sensors, respectively.

Aspect 55: The method of aspect 54, wherein the wound site is a chronic wound.

Aspect 56: The method of aspect 54, wherein the wound site is an acute wound.

Aspect 57: A composite electrode comprising: a moisture absorbing layer as in any one of aspects 1-14 that is connected to, or integrally formed with, an electrode as in any one of aspects 27-47.

Aspect 58: The composite electrode of aspect 57, wherein the moisture absorbing layer is co-planar with the electrode.

Aspect 59: The composite electrode of any one of aspects 57-58, wherein the moisture absorbing layer is entirely or partially surrounded by the electrode.

Aspect 60: The composite electrode of any one of aspects 57-59, wherein the moisture absorbing layer is a central moisture absorbing layer that is seated between a plurality of electrodes, the plurality of electrodes comprising a first electrode and a second electrode.

Aspect 61: The composite electrode of aspect 60, wherein the first and second electrodes are physically spaced apart, and electrically insulated, from one another by the central moisture absorbing layer.

Aspect 62: The composite electrode of any one of aspects 57-61, being fabricated by additive manufacturing, e.g., 3D printing.

Aspect 63: A method (e.g., a closed-loop method) for healing a wound site of a subject, the method comprising: applying the nanocomposite electrode of any one of aspects 57-62 over the wound site; and applying, by the electrodes, a series of electrical stimulations to the wound site until the wound site is healed, the series of electrical stimulations being based on a received impedance measurement from between the first and second electrodes.

Aspect 64: The method of aspect 63, wherein the first and second electrodes are in electrical communication with a control module that is operative to: receive the impedance measurement from between the first electrode and the second electrode; and, optionally, transmit, to a remote device, a signal corresponding to the impedance measurement.

The following Examples are for the purpose of illustration only and are not intended to limit the scope of the claims, which are appended hereto.

### Example 1

This Example describes experiments in which the inventors developed absorbent, flexible, transparent, and inexpensive substrate material using poly(vinyl alcohol) (PVA) as the host material for a variety of applications (referred to as “AFTIDerm” in the Examples). AFTIDerm samples of varying glycerol concentrations (1 wt %, 3 wt %, 5 wt %, 7 wt %, and 10 wt %) were fabricated and tested. The mechanical, electrical, thermal, absorption, and biological properties of AFTIDerm were evaluated both as a standalone substrate and when incorporated with carbon black (CB), the latter serving as a flexible electrode or integrated composite (referred to as “CB-AFTIDerm” in the Examples). It was surprisingly found that, at a 5 wt % glycerol concentration, AFTIDerm was stretchable and remained intact under torsion, demonstrating long-term mechanical stability, exhibited negligible absorption drop, and demonstrated an increase in absorption without swelling. Electrical testing unexpectedly demonstrated that 50% CB-AFTIDerm demonstrated a through thickness impedance of ˜10Ω and was stable over a one-week period when tested on an ischemic wound in a porcine chronic wound model. Benchtop testing and pre-clinical data validated AFTIDerm as a platform for use in epidermal electronics ranging from substrates to wound dressings and to wearable bioelectronics.

Methods

Materials

Poly(vinyl alcohol) (PVA) and glycerol were purchased from Sigma Aldrich (St. Louis, MO, USA) and used as received. CB was purchased from Cabot (CAS #1333-86-4; Boston, MA, USA) and used as received. Polypropylene petri dishes (Fischer Scientific #FB0875712, diameter 100 mm) were purchased and served to as the platform to cure the AFTIDerm. SPI Flash-Dry Silver epoxy was from Structure Probe, Inc. (West Chester, PA, USA). Carbon wire glue was purchased from Anders Products (Andover, MA, USA). The AWG30 size hook-up were from Remington Industry (Chicago, IL, USA). Impedance measurements were obtained using a GW-INSTEK LCR-821 meter (New Taipei City, Taiwan). For benchtop temperature assessments, Texas Instruments developer's kit (TMP117EVM) was used. A commercially available medical-grade silicone acrylate adhesive (2477P, 3M Inc.) adhered the AFTIDerm to the pig skin.

AFTIDerm, CB-PVA, and CB-AFTIDerm Fabrication

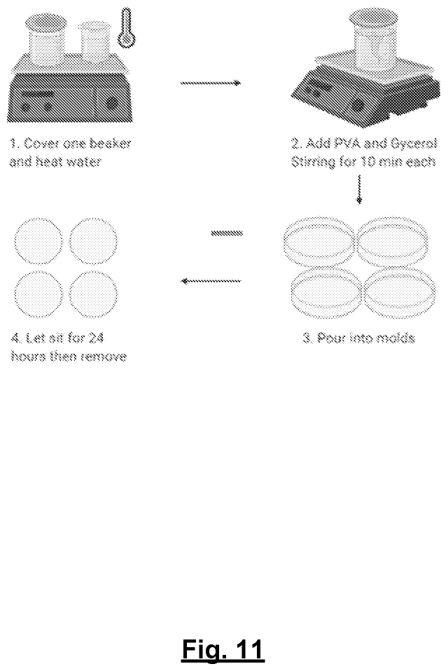

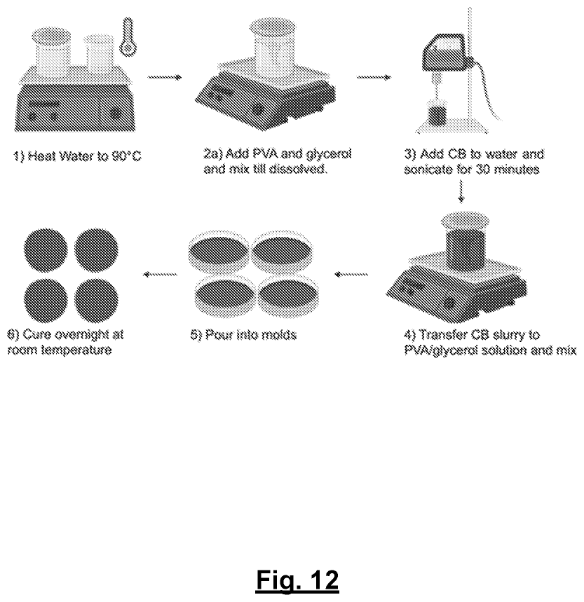

Each AFTIDerm sample regardless of glycerol concentration was fabricated in the same manner (FIG. 11). PVA was dissolved in 90° C. water under vigorous stirring. Glycerol of various weight percentages (1, 3, 5, 7, and 10 wt %) was added (masses relative to that of the water). CB-PVA was prepared in a similar manner except without the presence of glycerol. 50% CB was sonicated in water for 30 minutes (Qsonica Q500 probe, 500 W, 20 kHz, 30% duty cycle) and then added to the PVA solution and drop cast and left to cure for 24 hours. CB-AFTIDerm samples were prepared analogous to AFTIDerm and CB-PVA (FIG. 12). For clarity, AFTIDerm refers to substrates that contained PVA and glycerol. CB-PVA substrates only contained CB and PVA. CB-AFTIDerm substrates contained CB, PVA, and glycerol. All samples resulted in a homogenous thickness of 100 μm and were peeled from the dish and used for testing.

Contact Angle Testing of AFTIDerm Samples

Contact angle measurements were made by placing 10 μL of deionized water on each of the PVA composites of varied glycerol and CB concentrations respectively. For each sample, three samples were studied and 3 measurements for each sample were taken for statistical representation.

Mechanical Testing of AFTIDerm Samples

Standard uniaxial testing was applied to samples fabricated into a uniform rectangular shape and mounted to a custom-built Uniaxial Tensile Tester. One side of each sample was mounted to a fixed stage which was connected to a commercialized force sensor (DPM-3, Transducer Techniques) to monitor applied force. The opposite side of each sample was mounted to a screw-driven movable stage which has a stepper motor that was controlled by a LabVIEW program. The apparatus measured the applied force as the sample was elongated along its principal axis. Force measurements were made in increments of 5 μm at a frequency of 5 Hz, up to 20% applied strain. The instrument generated a force versus displacement curve for each test, and from this information, a stress versus strain curve is generated. Fitted line slopes of stress-strain curves were plotted to derive the Young's Moduli. In order to evaluate the mechanical stability of the AFTIDerm samples under periodic loading, a cyclic tensile test was performed on AFTIDerm samples of varied glycerol percentages. This test structure was subjected to cyclic loading over a strain range of 0 to 2.5% at 5 Hz. The test was performed using the previously described tensile tester for 50 identical cycles.

Thermal Diffusion Testing of AFTIDerm

The thermal diffusivity of AFTIDerm was evaluated to assess its ability to conduct and transfer heat (motivated by applications of this transparent substrate for monitoring skin temperature). Temperature was measured using a commercial medical grade temperature dye (TMP-117 EVM, Texas Instruments). The TMP-117 sensor was placed and secured on top of the AFTIDerm and the hot plate was set to 40° C., to mimic a physiologically relevant temperature. Temperature of the hot plate was measured without the AFTIDerm to serve as a control. Temperature was measured over a two-minute time period with 100 data points recorded per sample. This was repeated five times for each sample. The distribution in temperature among the two samples is presented for comparative purposes.

Scanning Electron Microscopy of CB-AFTIDerm

Scanning electron microscopy (SEM) was performed to evaluate the distribution of the CB particles along the AFTIDerm surface. CB-AFTIDerm was adhered to an aluminum stage (Ted Pella, #16202) via conductive carbon tape (Ted Pella, #16084-7).

Electrical Testing of CB-AFTIDerm Samples

The CB-AFTIDerm samples were wired using flash-dry silver epoxy (SPI FLASH-DRY Silver) to make pads on the electrode surface. After 15 minutes, AWG-20 jumper wires were placed on top of the pads. Carbon wire glue was used to hold wires in their places. After drying at room temperature for 24 hours, a further application of silver epoxy was used to enhance wire connection rigidity. Electrical contacts to the secured wires on each sample were made using standard probes. For resistance testing, rectangular samples 40 mm in length, 10 mm in width and 0.1 mm in thickness were fabricated using 50% CB. Standard, two-point, current-voltage (I-V) measurements were made on each sample using a DC power supply (Keysight E3631A) and a picoammeter (Keithley 6485). Each sample was secured to a glass slide to ensure that there was no stretching or flexing during I-V sweeps. The voltage was swept from 0 to 10 V in 0.1 V steps. The long-term electrical and thermal stability of the electrodes was measured both in the lateral and through thickness directions over a 25-hour period. Infrared thermography (FLIR) was used to record the temperature of the electrodes at the initial timepoint (hour 0) and final time point (hour 25). The lateral and through thickness resistance of the CB-AFTIDerm electrodes was quantified over a wide temperature range (22-50° C.). CB-AFTIDerm impedance was evaluated over a frequency range from 12 Hz to 100 KHz using an LCR meter (Instek LCR-821). A hand-held multimeter was used to measure the resistance at 1 Hz. Kelvin clips were used to make contacts with the CB-AFTIDerm samples and the LCR meter. Axelgaard-735 (AG-735), hydrogel electrode, was used as a control. Experiments were run in triplicate and data reported is mean±st. deviation.

Absorption Testing of AFTIDerm Compared to Standard of Care

AFTIDerm samples (with and without CB), Absorbent Tegaderm, HP Tegaderm, and Telfa (the latter three of which served as commercial controls) were immersed in phosphate buffer saline (PBS) solution, pH 7.4 and weighed at pre-set timepoints. Samples were taken out of the solution and weighed at hourly increments for the first 5 hours and 24 hours following for up to one week. Data reported is mean±st. deviation with six samples run per concentration group.

Adhesion Testing

CB-AFTIDerm adhesion to copper electrodes etched on a polyimide substrate were evaluated to assess the ability of the elastomeric nanocomposite to serve as a contact pad. Copper electrodes, etched from copper-clad polyimide substrates (Pyralux LF8530, 18 μm Copper, 75 μm polyimide), were fabricated using a photomask and conventional lithographical patterning. CB-AFTIDerm slurry was deposited on the etched electrodes (length 4 cm, width 1 cm) and left to cure at room temperature for 24 hours. The adhesion of the copper electrodes to the CB-AFTIDerm were evaluated using a custom-built force gauge. Samples were adhered to the stage via a silicone putty (Dowsil 737 Neutral Cure RTV Sealant). The vertical pull-off force was recorded. Experiments were run in triplicate and data reported is mean±st. deviation.

Biological Stability

The biological stability of the CB-AFTIDerm compared to CB-PVA, PVA, and AFTIDerm were studied over a one-week period. Changes in pH were quantified as a sharp decrease in pH would present a cytotoxic effect. Samples were placed in PBS and the pH of the supernatant was measured at each time interval. Experiments were run in triplicate and data reported is mean±st. deviation.

Pre-Clinical Evaluation

A porcine infected wound model was used to assess the efficacy of CB-AFTIDerm on compromised pig skin and the wound microenvironment. One female Yorkshire pig (30-35 kg) was housed prior to surgery in steel cages with a 12-hour light dark cycle. The animal was fed antibiotic-free food and water ad libitum throughout the study, Institutional Animal Care and Use Committee (IACUC) (VA #16-071-SW-16-009 and CWRU #: 2016-0331). The pig was observed for signs of infection or altered health at least 7 days prior to surgery. On the surgery day, the pig was sedated in the cage by intramuscular injection of Telazol, 3-4 mg/kg (Wyeth Pharmaceuticals, Madison, NJ, USA) and was then transferred to the operating suite and an airway was secured with endotracheal intubation. General anesthesia was then induced and the pig was placed in a prone position so that the entire dorsal region would be accessible for surgery. The back hair was shaved and six wound sites were marked over the paraspinal region using a prefabricated stencil. The pig's paraspinal region was then sterilely prepped with chlorhexidine scrub. The areas of skin to be excised were injected subcutaneously with a mixture of 1% lidocaine with 1:100,000 epinephrine (7cc at each excision site). Bilateral full thickness excisional wounds (6 cm diameter) were created. In order to create an ischemic wound, a sterile double-flanged silicone block (6 cm in diameter and 0.5 cm high) was placed into each wound and left in situ for 14 days. Each wound was covered with a Tegaderm™ dressing. The animal was wrapped in an elastic bandage (VetRap® 3M Health Care, St Paul, MN) to prevent animals interfering with the system. The pig was covered with a protective body jacket (Goat Tube®, Sullivan Supplies, Houston, TX) to prevent environmental contamination. The animal was awakened from general anesthesia, given post-operative. Following creation of each wound, 150 μL of a freshly cultured 0.5 McFarland solution of a green fluorescent protein labeled P. aeruginosa was evenly applied to each wound bed by pipette. This strain of bacteria was selected for initial testing because it is known to cause both acute and chronic infection, due to the formation of stable biofilms within the wound. An AFTIDerm composite comprised of CB-AFTIDerm and AFTIDerm was placed on two wounds and standard of care (Telfa) was placed on two wounds. Dressing changes for these wounds occurred on biopsy timepoint day (BTD) 1, 3, 5, 7, 10, 14, 21, and 28 with the composite and SOC bandage being discarded and replaced anew. Infrared thermography (FLIR, C2 Lepton 1101 9 Hz, serial #720146336), pH (Hanna Instruments #H199181), and camera images (Cannon EOS Rebel XSi) were taken at each timepoint to evaluate efficacy and healing.

Results—Material Characterization of AFTIDerm

The structure-property relationship of AFTIDerm (100 μm thickness) based on various glycerol percentages was first evaluated at different weight percentages of glycerol (Table 2).

TABLE 2
Material properties of AFTIDerm at
varying glycerol concentrations

| Glycerol | Young's Modulus | Cyclic Stress | Water Contact Angle |
|---|---|---|---|
| (wt %) | (MPa) | (kPa) | (°) |
| 0 | 18.4 ± 0.12 | 1168 ± 338 | 53.3 ± 14 |
| 1 | 18.9 ± 0.12 | 761.0 ± 218 | 35.0 ± 6.7 |
| 3 | 9.37 ± 0.036 | 468.0 ± 99.7 | 30.6 ± 5.1 |
| 5 | 7.52 ± 0.021 | 380.7 ± 45.5 | 29.7 ± 2.2 |
| 7 | 6.39 ± 0.015 | 230.5 ± 55.2 | 34.2 ± 4.4 |
| 10 | 2.98 ± 0.0077 | 121.5 ± 30.1 | 34.2 ± 5.7 |

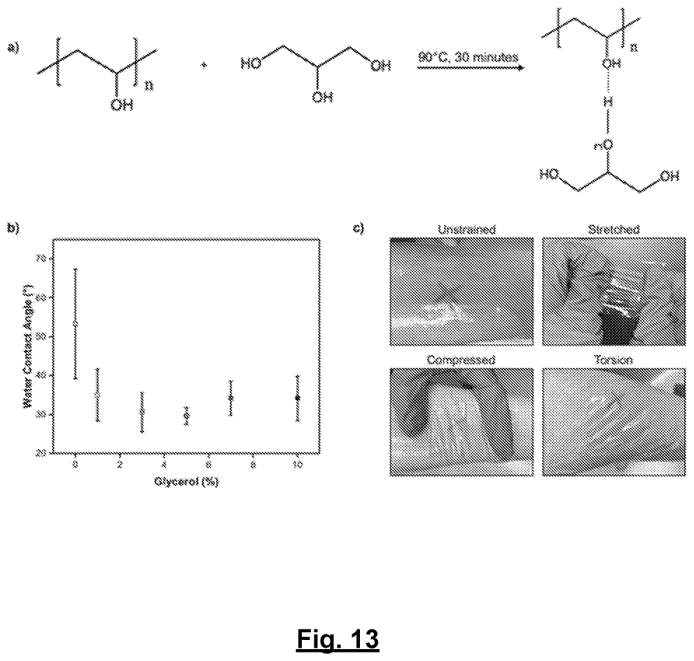

AFTIDerm was fabricated in a one pot synthesis (FIG. 13(a)). It was surprisingly found that: (1) the introduction of glycerol into a PVA hydrogel provides AFTIDerm with thermoplasticity, self-healing, and long-term moisture retention and increase its low-temperature tolerance; (2) 3% (w/v) PVA provides a flexible AFTIDerm substrate (results not shown); (3) the water contact angle demonstrated an increase in AFTIDerm hydrophilicity up to 5 wt % glycerol, with an insignificant increase noted at 7% and 10% (FIGS. 13(b)); and (4) AFTIDerm at 5 wt % glycerol was stretchable and remained intact under torsion (FIG. 13(c)).

Mechanical Testing

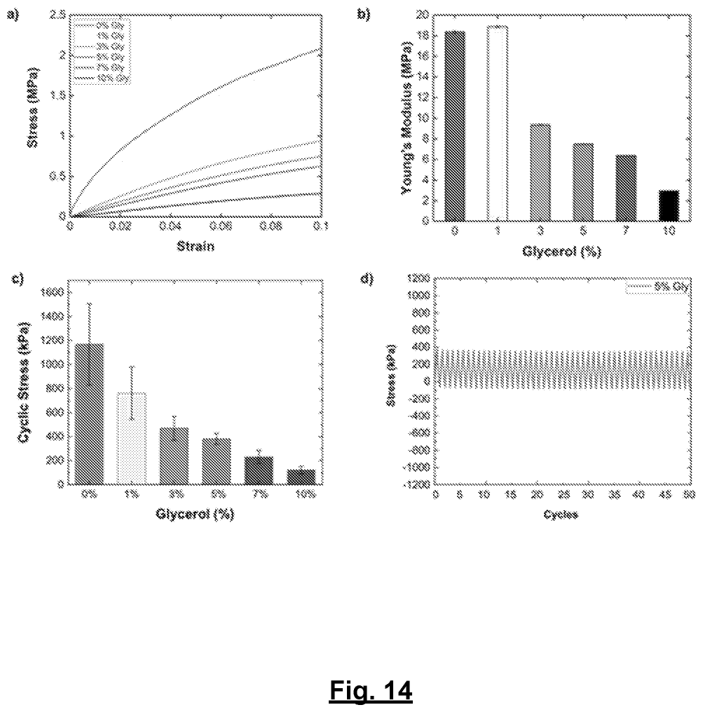

Young's Modulus was found to decrease with increasing glycerol concentration (r²=0.70 modeled by an exponential regression) and cyclic stress (r²=0.95 modeled by an exponential regression) (FIGS. 14(a)-(d)). Thus, an increase in glycerol concentration had an inverse effect on the mechanical properties.

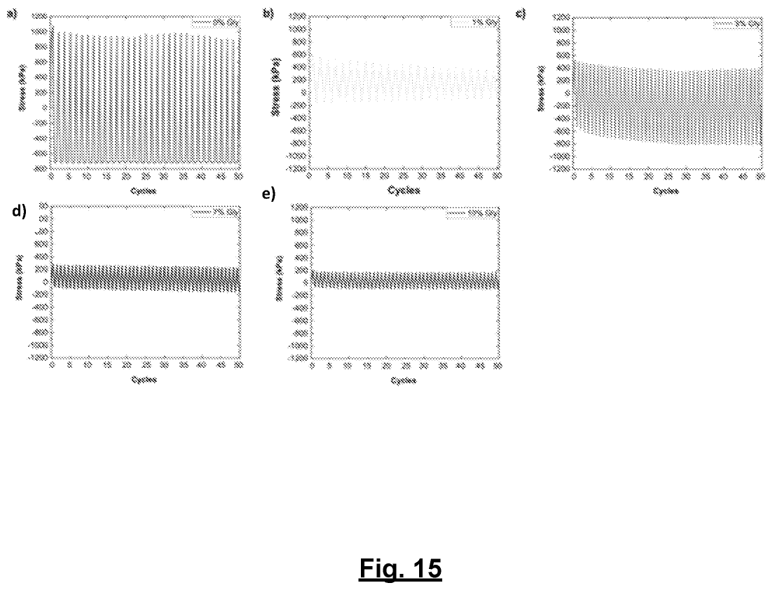

Long-term mechanical actuation of AFTIDerm at a 5 wt % glycerol demonstrated no hysteresis with a cyclic stress of 380.7±45.5 kPa. The cyclic stress of AFTIDerm at varied glycerol concentrations apart from 5 wt % was also investigated (FIGS. 15(a)-(e)). Concentrations below 5 wt % experienced hysteresis on long-term testing. Data suggests that glycerol percentages above 5 wt % provide long-term mechanical stability.

Absorption Testing

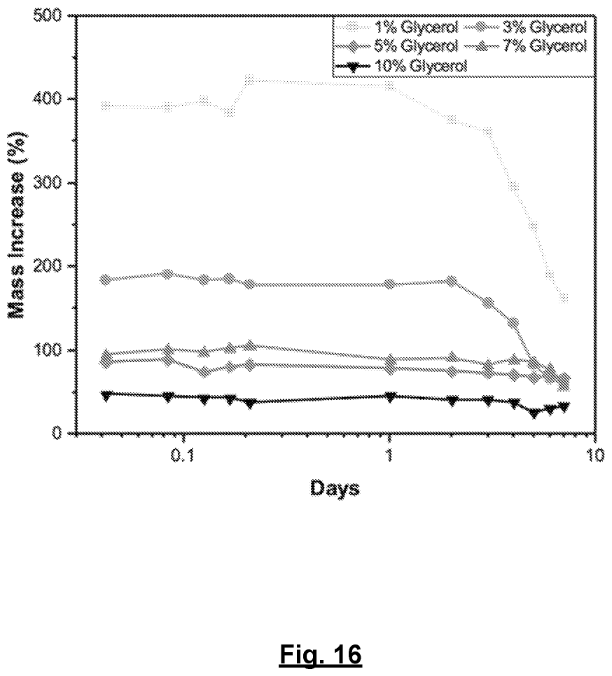

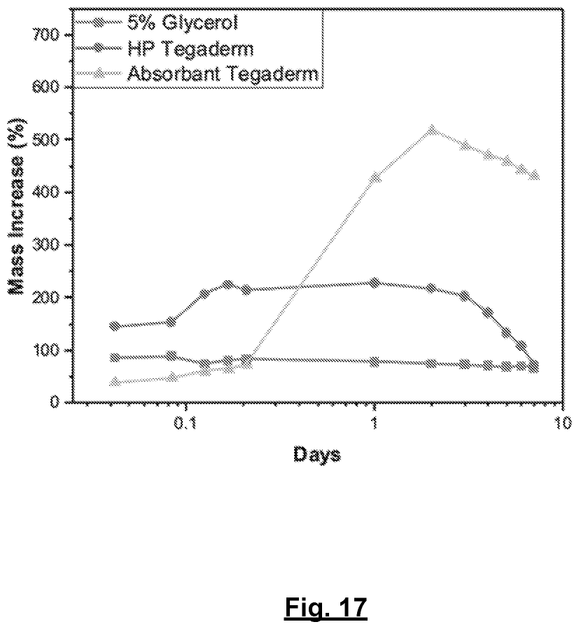

AFTIDerm absorptive properties at various glycerol concentrations were evaluated b (FIG. 16) and compared to industry standards HP Tegaderm and Absorbent Tegaderm (FIG. 17; Table 3).

TABLE 3
Absorption data comparing AFTIDerm at varied glycerol percentages (wt %) against
HP Tegaderm and Absorbent Tegaderm (data reported as mean ± st. dev.; n = 6)

| | | 1% | 3% | 5% | 7% | 10% | HP | Absorbent |
|---|---|---|---|---|---|---|---|---|
| | Time | Glycerol | Glycerol | Glycerol | Glycerol | Glycerol | Tegaderm | Tegaderm |
| Hours | 1 | 392 ± 140 | 184 ± 29 | 86.2 ± 38 | 94.6 ± 57 | 47.5 ± 23 | 145 ± 23 | 39.1 ± 8.91 |
| | 2 | 390 ± 150 | 190 ± 30 | 89.1 ± 47 | 101 ± 49 | 45.6 ± 16 | 153 ± 28 | 48.4 ± 15 |
| | 3 | 398 ± 120 | 184 ± 36 | 74.4 ± 44 | 97.1 ± 61 | 43.3 ± 19 | 207 ± 37 | 60.5 ± 12 |
| | 4 | 384 ± 84 | 185 ± 36 | 80.5 ± 43 | 102 ± 70. | 43.0 ± 13 | 225 ± 51 | 65.7 ± 20. |
| | 5 | 423 ± 150 | 178 ± 62 | 82.9 ± 55 | 106 ± 60 | 38.2 ± 17 | 215 ± 41 | 72.7 ± 15 |
| Days | 1 | 416 ± 130 | 178 ± 46 | 78.5 ± 41 | 88.4 ± 57 | 45.3 ± 23 | 228 ± 48 | 427 ± 180 |
| | 2 | 375 ± 78 | 182 ± 45 | 74.7 ± 41 | 91.2 ± 62 | 40.9 ± 18 | 217 ± 71 | 519 ± 140 |
| | 3 | 361 ± 110 | 157 ± 52 | 72.8 ± 43 | 82.5 ± 56 | 40.6 ± 20. | 203 ± 110 | 490. ± 150 |
| | 4 | 295 ± 110 | 133 ± 40 | 70.9 ± 40 | 88.8 ± 55 | 37.7 ± 20 | 171 ± 110 | 471 ± 160 |
| | 5 | 248 ± 22 | 82.9 ± 88 | 68.1 ± 38 | 85.9 ± 63 | 26.0 ± 21 | 133 ± 110 | 460. ± 180 |
| | 6 | 189 ± 200 | 65.5 ± 76 | 69.9 ± 37 | 77.5 ± 50 | 30.6 ± 22 | 109 ± 100 | 444 ± 200 |
| | 7 | 162 ± 170 | 60.3 ± 63 | 67.4 ± 45 | 57.3 ± 51 | 33.4 ± 20. | 73.3 ± 76 | 432 ± 210 |

Glycerol concentrations below 5 wt % exhibited a significant drop in mass increase from day 2 to day 7. The same was observed for both HP Tegaderm and Absorbent Tegaderm (latter two use as commercial controls commonly utilized for wound care). This suggests that these materials reached their maximum absorption capacity prior to day 2. Surprisingly, absorption drop was negligible for glycerol concentrations greater than 5 wt %; thus, suggesting 7% and 10% as ideal glycerol concentrations as well for AFTIDerm.

Thermal Characterization

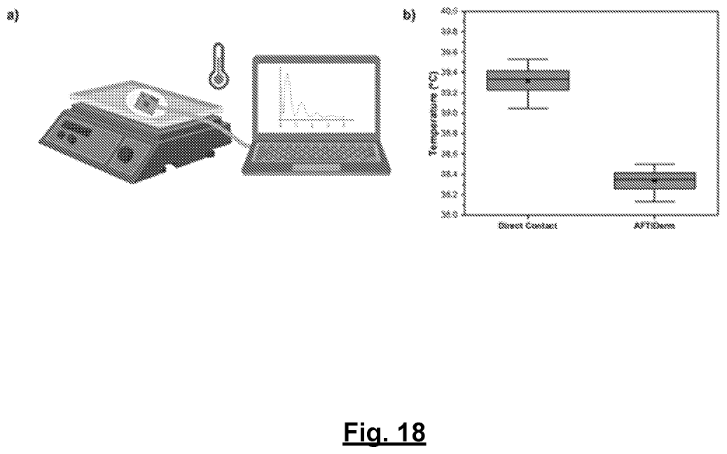

Thermal diffusion was studied to assess the ability of AFTIDerm to conduct heat through the bulk interface using the TMP 117 sensor (FIG. 18(a)). The temperature difference was found to be 0.9° C., well within a clinically-relevant range (FIG. 18(b)). The mean temperature measured by direct contact of the temperature sensor to the hotplate was 39.2° C. compared to 38.3° F. when placed on the AFTIDerm surface. Despite this negligible difference, the data suggests that the AFTIDerm enables the diffusion of heat from the hot plate to the sensor thereby enabling measurement of temperature within a physiologically relevant window.

Results—Pre-Clinical Evaluation of AFTIDerm

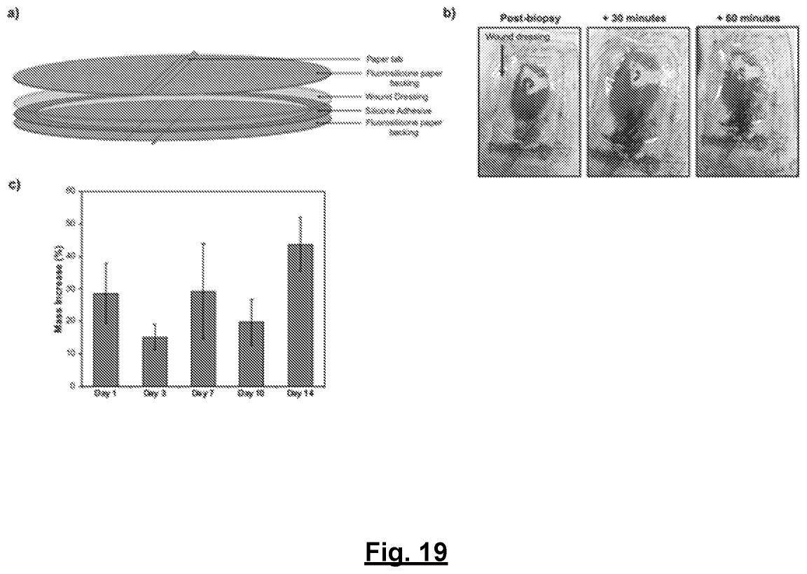

AFTIDerm was evaluated as an absorbent wound dressing (FIG. 19(a)). When placed over the wound in a porcine chronic wound model, 5 wt % glycerol AFTIDerm substrate absorbed exudate from the wound without swelling over a one-hour period (FIG. 19(b)). Over a 14-days testing in the same model, AFTIDerm demonstrated a ˜44% increase in absorption without swelling (FIG. 19(c)).

Results—Material Characterization of CB-AFTIDerm

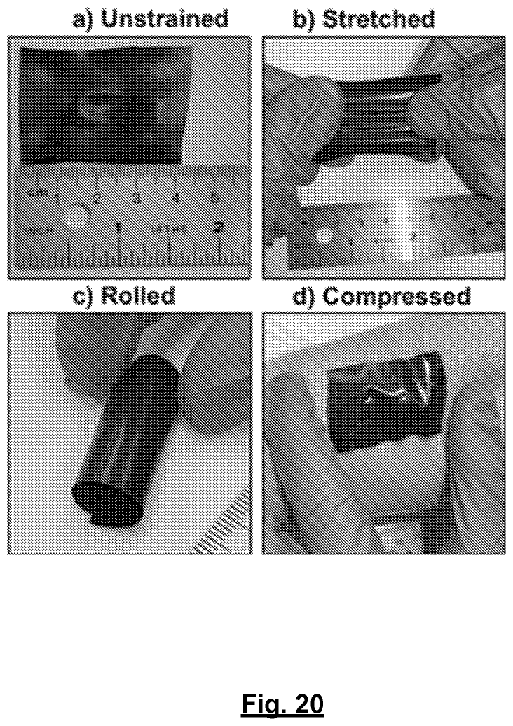

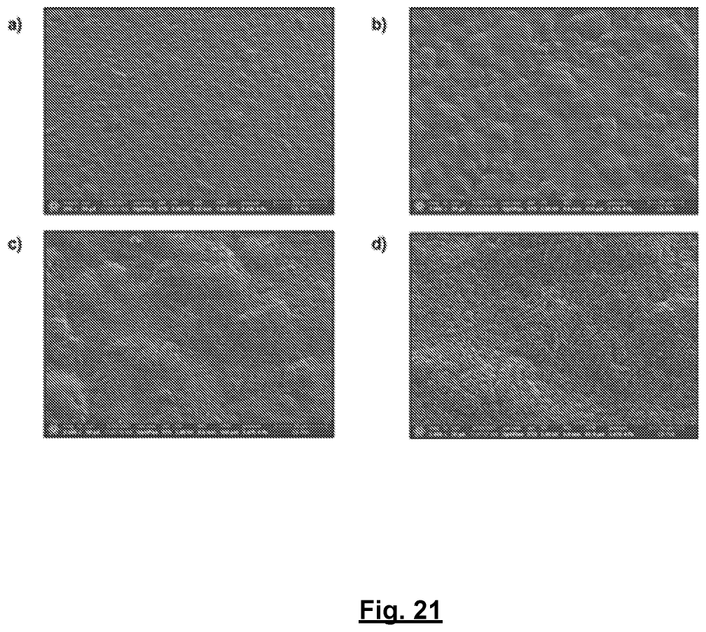

It was surprisingly found that 50% CB resulted in maximum conductivity of CB-AFTIDerm electrodes (FIGS. 20(a)-(d)). Electrodes (4 cm length, 1 cm width, 100 μm thickness) were fabricated leveraging the AFTIDerm fabrication process (described above). Scanning electron microscopy (SEM) revealed the distribution of the CB particles over the AFTIDerm surface (FIG. 21).

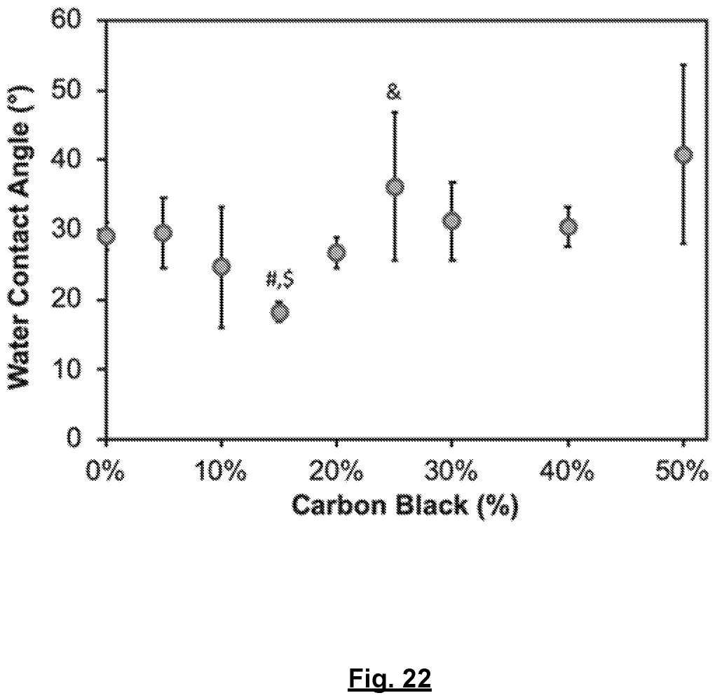

The water contact angle was evaluated to assess changes in the hydrophilicity of the PVA surface based on varied CB concentrations (FIG. 22). As mentioned earlier, we unexpectedly found that that 5 wt % glycerol (water contact angle: 29.1±2.1°) created a stable crosslinked PVA substrate due, in part, to hydrogen bonding between the hydroxyl groups on the PVA and glycerol, respectively. An increase in hydrophilicity (decrease in contact angle from 29.6±5.0° to 18.2±1.4°) was observed from 0 wt % to 15 wt % CB. A statistically significant decrease in hydrophilicity (increase in contact angle from 18.2±1.4° to 31.2±5.6°) was observed with increasing CB concentrations from 15 to 30 wt %. The data overall suggests a negligible change in contact angle from 25 wt % to 50 wt % (36.2±10.6° to 40.8±12.8°) thereby suggesting the surface chemistry of the electrode is unaffected by changes in CB addition.

Electrical Stability Testing

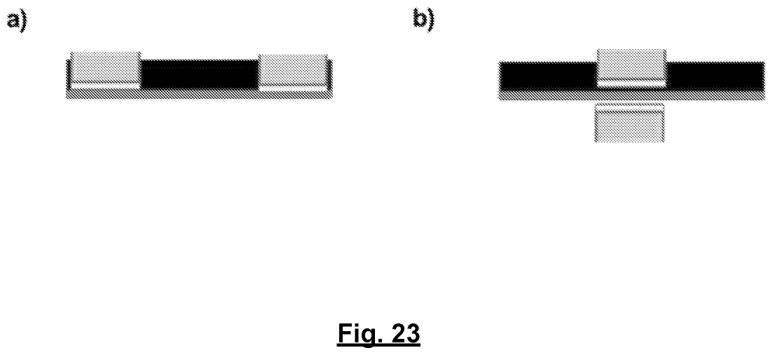

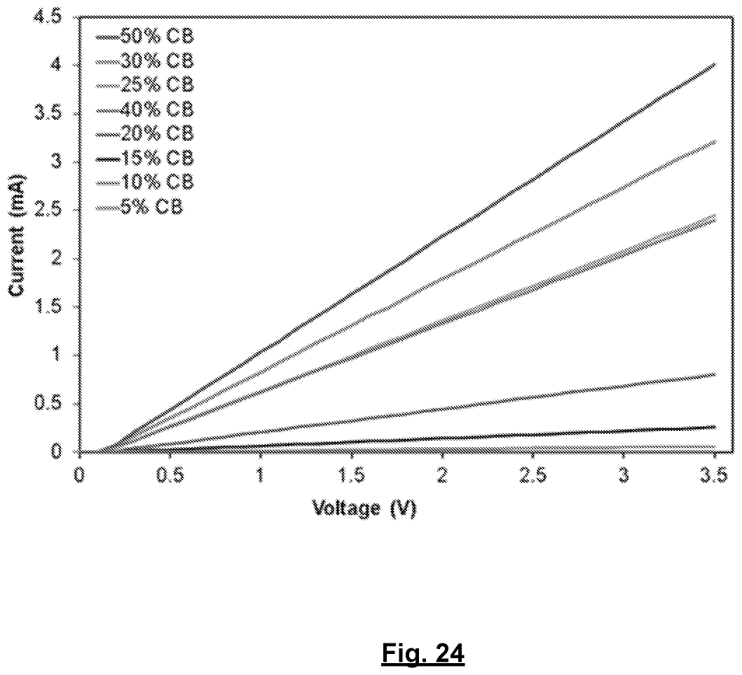

The electrical performance of the CB-AFTIDerm electrode was tested in the lateral and through thickness directions (FIGS. 23(a)-(b)). A representative plot of an I-V sweep for composite samples from each of the CB concentration groups shows that the samples exhibit ohmic behavior (FIG. 24).

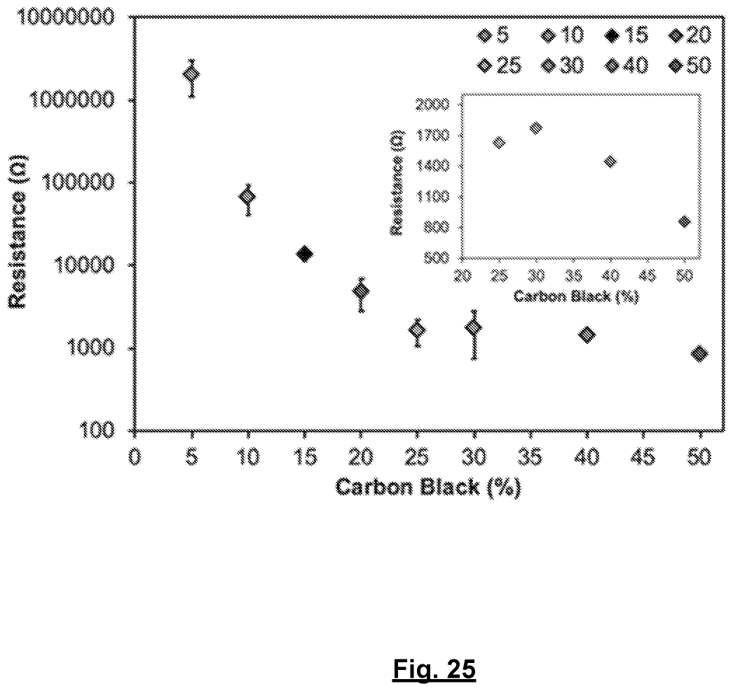

Resistance values (5%: 2.03±0.9 MΩ, 10%: 66.9±2.7 kΩ, 15%: 13.4±2 kΩ, 20%: 4.9±2.0 kΩ, 25%: 1.63±0.57 kΩ, 30%: 1.78±0.1 kΩ, 40%: 1.44±0.17 kΩ, and 50%: 859±105Ω) demonstrated an exponential decay (r²=0.93) as a function of increasing CB concentration (FIG. 25).

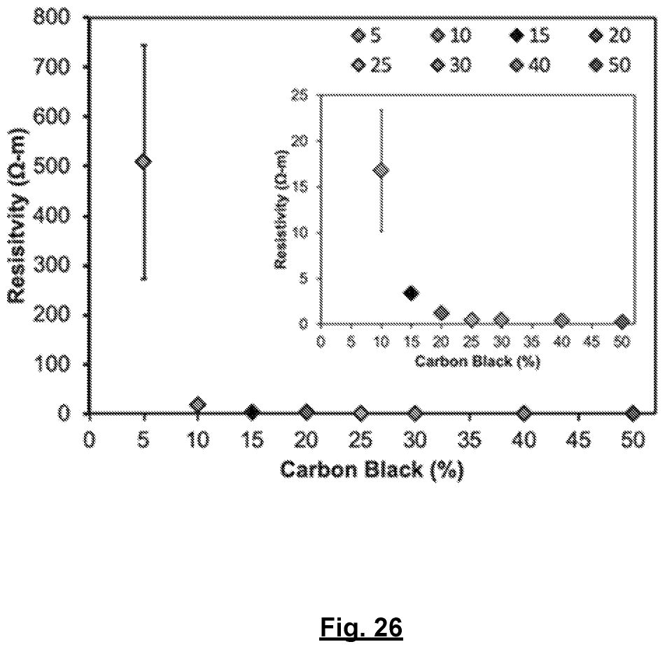

Resistivity (5%: 509±236 Ω-m, 10%: 16.7±6.7 Ω-m, 15%: 3.37±0.51 Ω-m, 20%: 1.22±0.50 Ω-m, 25%: 0.407±0.14 Ω-m, 30%: 0.443±0.25 Ω-m, 40%: 0.36±0.04 Ω-m, and 50%: 0.215±0.026 Ω-m) followed the similar trend (r²=0.93) (FIG. 26). The conductivity of the electrodes is predicated on the interaction and conductive pathways formed between the CB molecules. Our data suggests that at lower CB concentrations, such as at 5 wt % the statistical likelihood of conductive paths formed in the composite is less, thereby causing greater variance when near threshold. Interestingly, our data shows significantly improved electrical characteristics of the nanocomposite when the CB concentration is or exceeds 25 wt %. Other data (not shown) also shows that exceeding CB concentrations beyond 50 wt % (e.g., >60 wt %) lead to deleterious changes in the mechanical properties of the composite (flexible to “brittle” state).

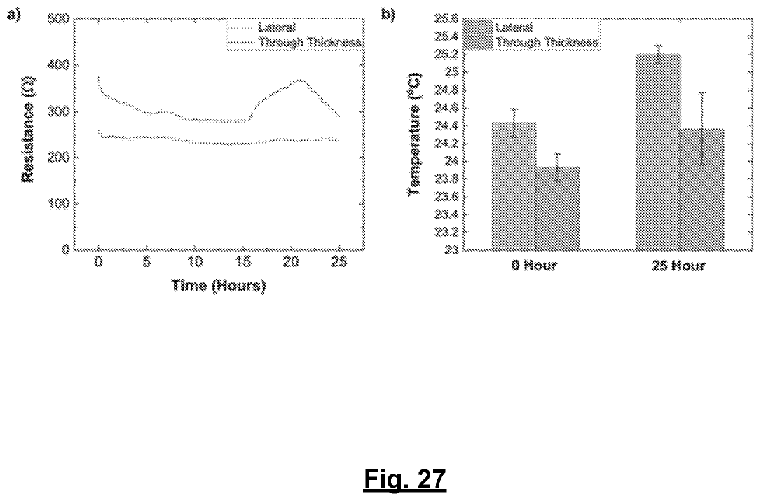

CB-AFTIDerm electrode electrical stability was evaluated over a 25-hours (FIGS. 27(a)-(b)). Resistance was evaluated in the lateral and through thickness directions. Mean lateral resistance over the 25-hour period varied by ˜32Ω (226.4Ω to 258.4Ω). Mean through thickness resistance over the 25-hour period varied by ˜98.2Ω (278.9Ω 377.1Ω). Variations in resistances between the lateral and through thickness directions (˜66.2Ω) can be attributed to CB particles agglomeration within the bulk AFTIDerm. The thermal profiles of CB-AFTIDerm electrodes were assessed before and after electrical actuation to quantify heat loss during testing (FIG. 27(a)). Presence of heat generation is common in direct current actuation. Surface electroceutical therapy can cause deleterious effects, such as erythema or non-specific cathodal vasodilation on the skin surface if excess heat is generated. Advantageously, over a 25-hour period, negligible temperature changes of 0.5° C. and 0.8° C. was found in the lateral and through thickness directions (FIG. 27(b)).

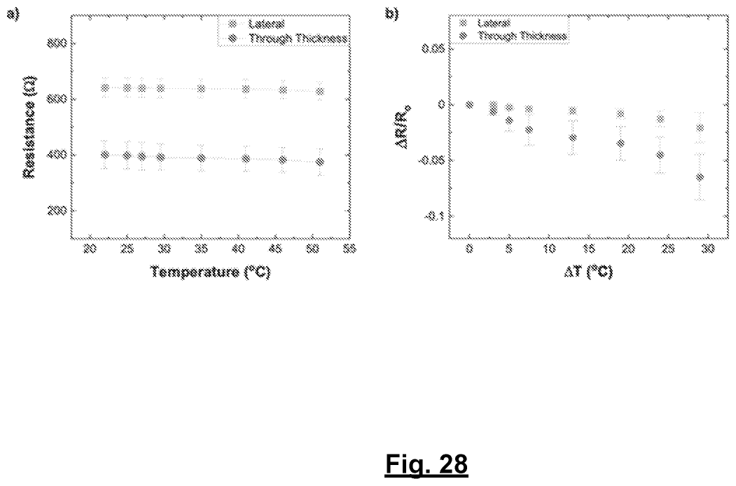

The thermal stability of CB-AFTIDerm electrodes was evaluated over a temperature range from 21° C. to 51° C. (FIGS. 28(a)-(b)). Both the lateral and through thickness directions remained relatively homogenous with a slightly negative temperature coefficient of resistance.

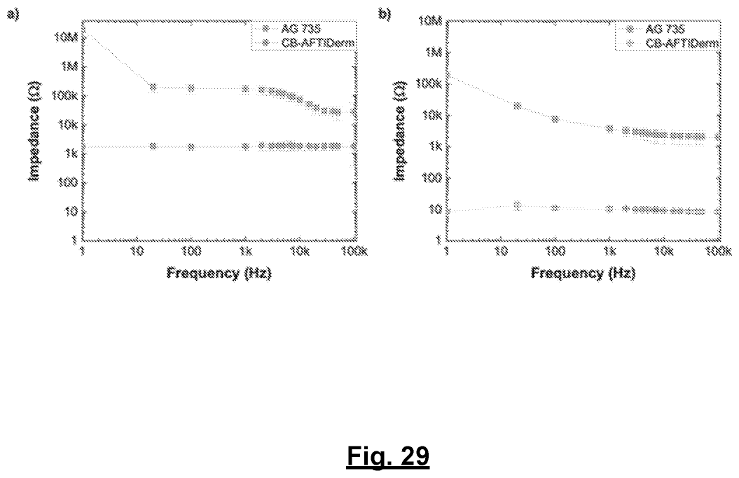

The impedance of the CB-AFTIDerm was assessed over frequency range of 1 Hz to 100 KHz and compared against a commercial hydrogel electrode, Axelgaard-735 (FIGS. 29(a)-(b); Table 4).

TABLE 4
Lateral and through thickness impedance of AG-735 and CB-AFTIDerm electrodes
(experiments performed in triplicate, data presented as mean ± st. dev)

| | | | | | CB-AFTIDerm/ |
|---|---|---|---|---|---|
| | | AG-735 | | CB-AFTIDerm | Conductive Tape |
| Frequency | AG-735 | (Through | CB-AFTIDerm | (Through | (Through |
| (Hz) | (Lateral Ω) | Thickness Ω) | (Lateral Ω) | Thickness Ω) | Thickness Ω) |
| 1 | 1.96E7 ± 13860800 | 193333 ± 55075 | 1744 ± 498 | 8.5 ± 0.7 | 569.0 ± 182 |
| 20 | 193333 ± 73339 | 19946 ± 3939 | 1799 ± 431 | 13.7 ± 4.1 | 496.0 ± 155 |
| 100 | 183000 ± 66685 | 7550 ± 1275 | 1722 ± 435 | 11.2 ± 1.1 | 448.0 ± 181 |
| 1000 | 172667 ± 60692 | 3800 ± 1081 | 1756 ± 483 | 10.4 ± 1.3 | 374.0 ± 103 |
| 2000 | 161333 ± 56083 | 3256 ± 1137 | 1893 ± 664 | 10.8 ± 0.4 | 356.3 ± 106 |
| 3000 | 147000 ± 49487 | 2986 ± 1147 | 1817 ± 580 | 10.0 ± 0.6 | 337.3 ± 103 |
| 4000 | 132000 ± 43485 | 2809 ± 1155 | 1808 ± 542 | 9.9 ± 0.9 | 326.0 ± 100 |
| 5000 | 118667 ± 38553 | 2643 ± 1162 | 1888 ± 646 | 10.0 ± 0.9 | 317.0 ± 95.8 |
| 6670 | 99333 ± 31895 | 2508 ± 1174 | 1948 ± 771 | 9.8 ± 0.5 | 309.0 ± 99.9 |
| 7500 | 91667 ± 28937 | 2396 ± 1158 | 1824 ± 543 | 9.5 ± 0.7 | 305.0 ± 95.5 |
| 10,000 | 72757 ± 23620 | 2324 ± 1138 | 1813 ± 549 | 9.3 ± 0.6 | 298.0 ± 95.5 |
| 15,000 | 50500 ± 16889 | 2250 ± 1082 | 1787 ± 530 | 9.0 ± 0.4 | 298.7 ± 86.2 |
| 20,000 | 38100 ± 14823.29 | 2188 ± 1069 | 1719 ± 473 | 8.9 ± 0.7 | 302.3 ± 80.0 |
| 29,000 | 29967 ± 8863 | 2132 ± 1028 | 1796 ± 522 | 8.7 ± 1.1 | 304.3 ± 76.7 |
| 40,000 | 28907 ± 10951 | 2084 ± 995 | 1819 ± 552 | 8.5 ± 1.4 | 285.7 ± 85.7 |
| 50,000 | 26853 ± 13317 | 2062 ± 985 | 1812 ± 481 | 8.5 ± 1.3 | 276.3 ± 85.1 |
| 100,000 | 28617 ± 28247 | 2005 ± 947 | 1774 ± 699 | 8.5 ± 1.1 | 268.3 ± 85.1 |

The CB-AFTIDerm electrodes exhibited near uniform behavior, whereas the AG-735 electrodes decreased in impedance with increase in frequency range, the drop in through thickness impedance can be attributed to the packing of the CB within the AFTIDerm bulk.

Assessing Relationship Between Adhesion and Impedance

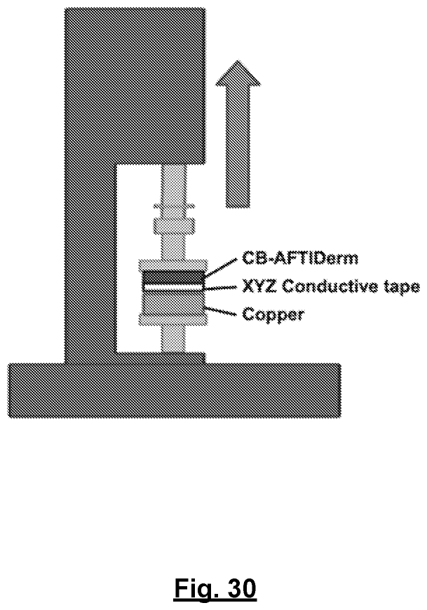

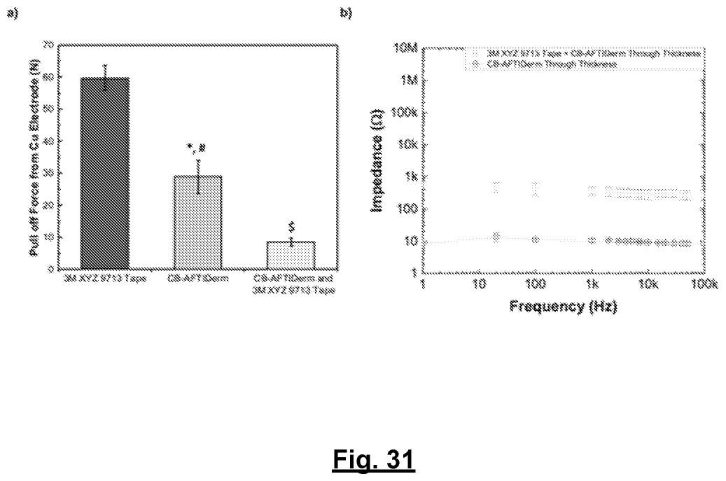

CB-AFTIDerm was assessed as a contact pad when adhered to a copper-clad polyimide substrate. Typical adhesion mechanisms involving elastomeric nanocomposites to copper involve either surface modification of the host polymer (e.g., polydimethyl siloxane), plasma treatment, or the use of a double-sided conductive adhesive. Surface modification involves the use of solvents and chemistries, such as thiol-epoxy reactions, which if not removed completely, could pose deleterious reactions from a biocompatibility standpoint and poses challenges from a scalability standpoint. The latter, involving the use of a conductive tape, while improving device integrity, adds to the overall through thickness impedance of the system. Towards achieving a scalable and fully biocompatible system, the adhesion between the CB-AFTIDerm electrode to that of a copper electrode (etched with the same dimensions as the nanocomposite) was assessed via the use of the double-sided conductive tape and when directly cured on the copper electrode (deposited via drop casting) (FIG. 30). The adhesion of the tape to the electrode served as the control. The pull-off force of the tape from the copper electrode was 59.7±3.9 N, CB-AFTIDerm and copper electrode was 28.8±5.1 N, and CB-AFTIDerm/conductive tape to the copper electrode was 8.5±1.3 N (FIG. 31(a)). Correlating this data to the through thickness impedance over the frequency range studied showed that the presence of the conductive tape constituted 94.5% of the overall through thickness of the combined interfaces (copper electrode, conductive tape and CB-AFTIDerm electrode mean impedance of 345.4Ω, and CB-AFTIDerm electrode mean impedance of 9.72Ω) (FIG. 31(b)). Thus, the direct curing of CB-AFTIDerm slurry onto the copper electrode (and curing at room temperature overnight), which contributed only 5.5% of the total through thickness impedance, was surprisingly found to be an ideal method to fabricate conductive nanocomposite contact pads that interface with flexible substrates.

CB-AFTIDerm Absorption Testing

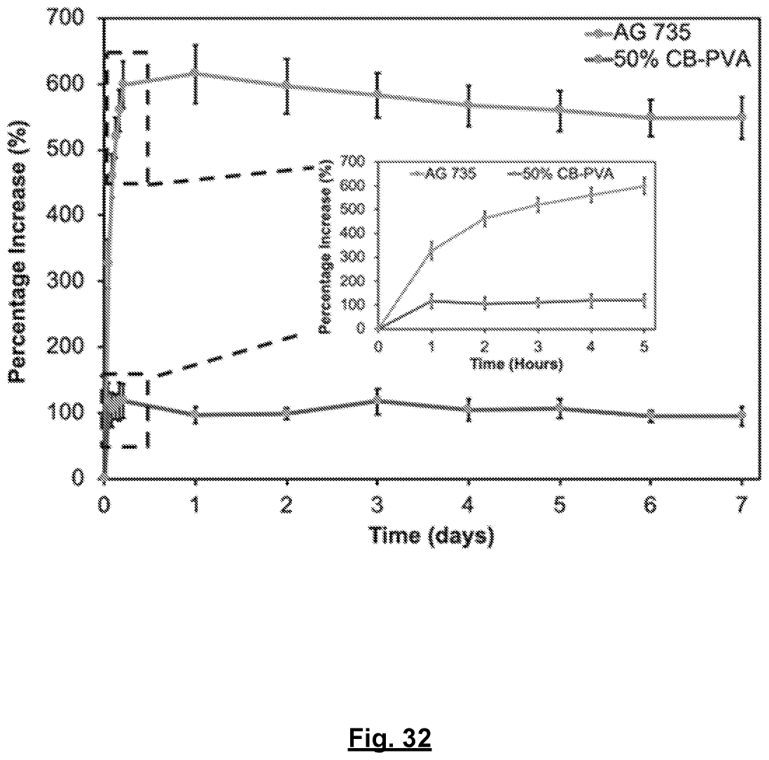

Absorption data demonstrated a significant uptake in mass for the AG-735 hydrogel electrode (615±44.8%) compared to that observed by the 50% CB-PVA (96.8±12.7%) over the initial 24-hour period (FIG. 32). At the one-week mark, the AG-735 electrode exhibited a mass increase of 548.5±30.9% compared to 94.9±14.2% mass increase of the 50% CB-PVA electrode. Compared to the AG-735 which exhibited a net difference of ˜66.5% over the one-week timeframe, the 50% CB-PVA electrode demonstrated a net uptake of only 1.9%. This suggests that the addition of a hydrophobic active agent, such as CB did not alter the hydrophilic absorbent capabilities of the PVA and enabled the electrode to hold its mass over a one-week time frame. The one-week period was selected as devices used for ambulatory monitoring or current hydrogel adhesives are worn on the skin for that time frame.

Leachability Testing

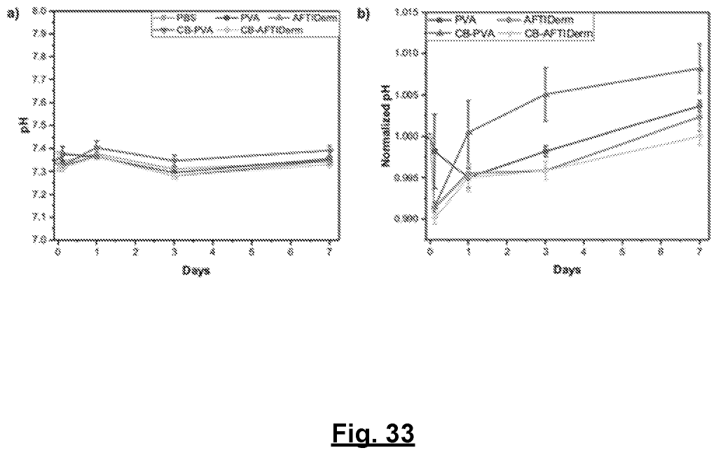

Towards assessing their clinical utility and effect of glycerol leachability from the host material, the biological stability, indicative of the relative change in pH when immersed in PBS, of AFTIDerm and CB-AFTIDerm were evaluated over a one-week period, with intermediate timepoints reflecting pre-clinical benchmarks (Day 0, 1, 3, and 7) followed in our animal studies (FIGS. 33(a)-(b)). No statistical difference was noted among the differences in pH between the various samples (Table 5).

TABLE 5
pH of PBS, PVA, AFTIDerm, CB-PVA, and CB-AFTIDerm over a one-week period
(experiments run in triplicate; data reported as mean ± st. dev.)

| Days | PBS | PVA | AFTIDerm | CB-PVA | CB-AFTIDerm |
|---|---|---|---|---|---|
| 0 | 7.34 ± 0.041 | 7.34 ± 0.041 | 7.34 ± 0.041 | 7.34 ± 0.041 | 7.34 ± 0.041 |
| 0.125 | 7.34 ± 0.041 | 7.38 ± 0.033 | 7.33 ± 0.0047 | 7.33 ± 0.0047 | 7.32 ± 0.0047 |
| 1 | 7.38 ± 0.017 | 7.36 ± 0.0094 | 7.37 ± 0.0047 | 7.40 ± 0.029 | 7.36 ± 0.012 |
| 3 | 7.31 ± 0.0082 | 7.30 ± 0.0047 | 7.28 ± 0.0 | 7.35 ± 0.024 | 7.28 ± 0.0082 |
| 7 | 7.35 ± 0.017 | 7.36 ± 0.0047 | 7.35 ± 0.013 | 7.39 ± 0.022 | 7.33 ± 0.0082 |

The pH found among the samples confirms their stability with and without glycerol and CB. The testing performed was representative of that expected in ISO-10993-14.

Composite Absorption Testing

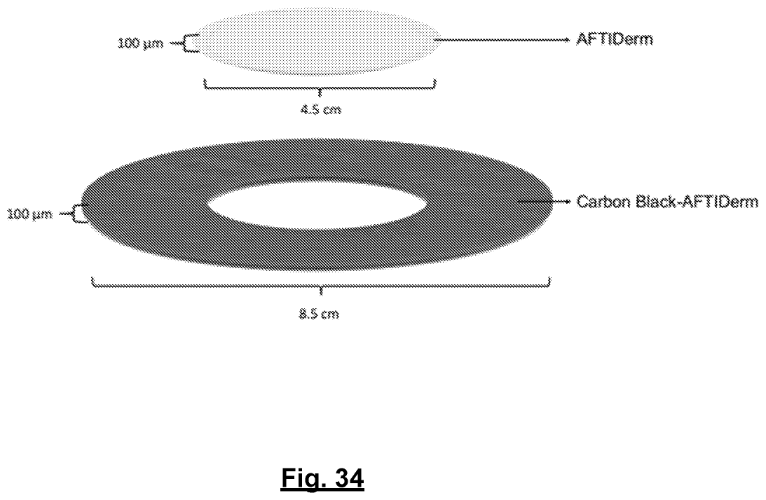

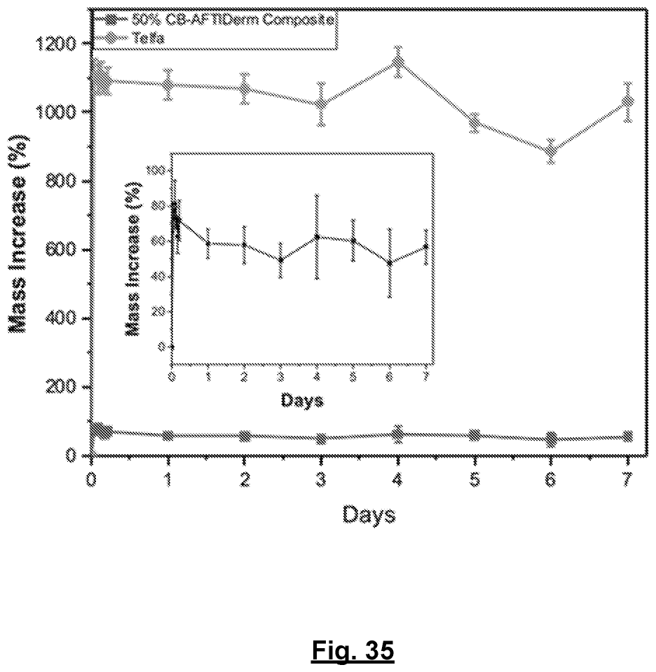

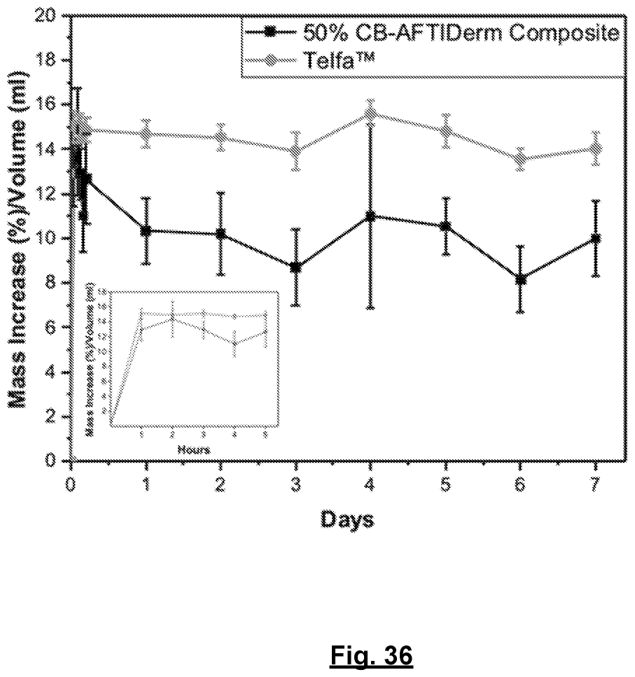

A composite composed of AFTIDerm in the center (diameter 4.5 cm) with CB-AFTIDerm encompassing it was created to assess the multi-material integration between the two (FIG. 34). Such composites have potential application in wound dressings where clinicians desire to view the healing of the wound without removal of the bandage. A 7-day benchtop absorption study of the composite was performed comparing the absorption capabilities against Telfa, a dressing commonly used for wound care applications (FIGS. 35-36). Over a one-week span compared to Telfa, the composite remained relatively stable in mass absorption (data not shown).

Results—Pre-Clinical Evaluation of CB-AFTIDerm Composite

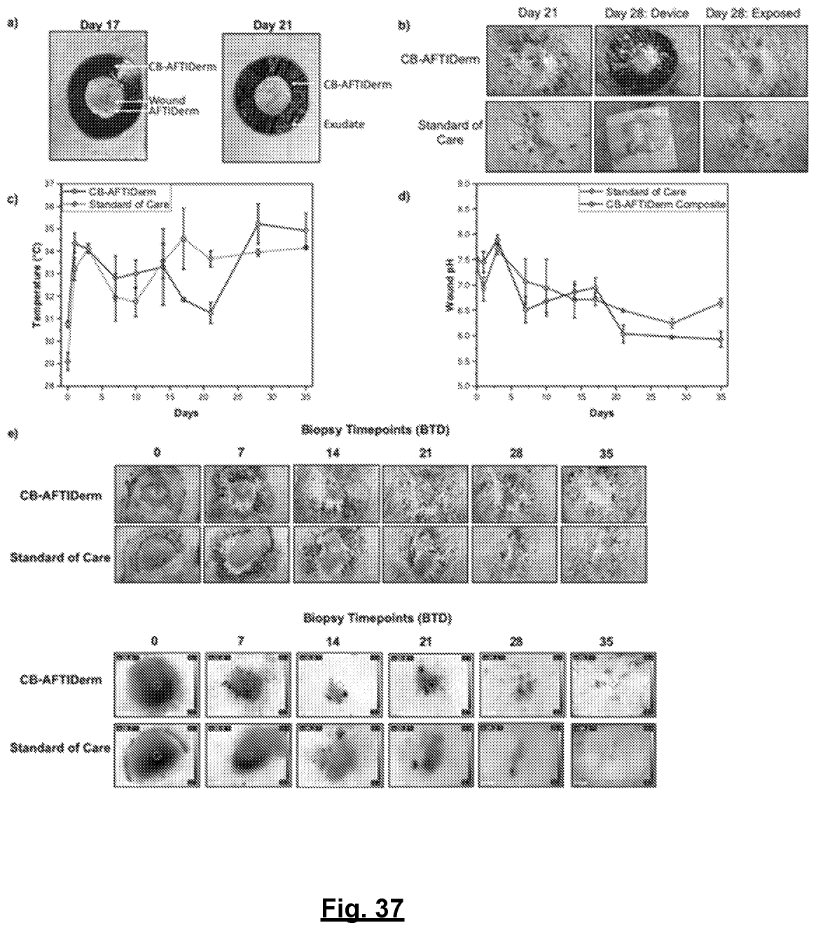

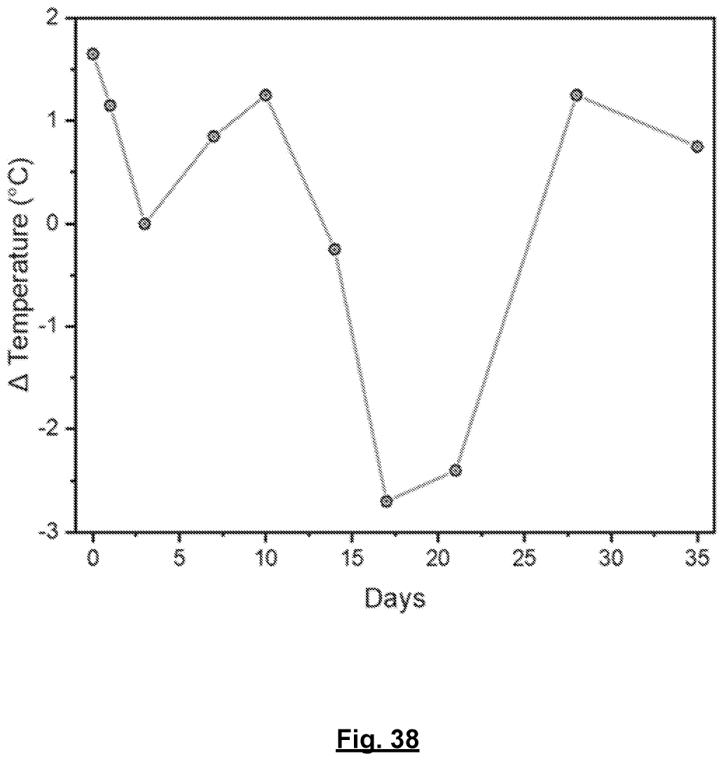

The biocompatibility of the CB-AFTIDerm composite was assessed when placed on a chronic ischemic wound of a Yorkshire pig over a 35-day period (FIGS. 37(a)-(e)). The composite and a commercial wound dressing deemed standard of care (e.g., Telfa) was placed on the wound at pre-determined clinical timepoints, left on the wound till the next change, and discarded. Infrared thermography and pH measurements were used to qualitatively and quantitatively assess materials compatibility on the stratum corneum. Qualitative adjudication from Day 17 to day 21 demonstrated the absorption of wound exudate by the dressing. Over a one-week period (day 21 to 28), the composite did not exhibit any deleterious reactions on the skin, indicative of erythema. The change in temperature between the composite and Telfa demonstrated that the CB-AFTIDerm did not result in an increase in wound temperature, indicative of a foreign body response, infection, or adverse reaction when placed over compromised skin. The difference in temperature between the composite and Telfa wound dressing showed differences within a clinically appropriate window (FIG. 38). The change in pH over the 35-day period demonstrated that the CB-AFTIDerm composite did not hamper re-epithelization based on the drop in pH over the clinical time course. The presence of the composite provided a moist and occlusive environment, which has been well documented to assist in the healing process, to aid in healing compared to the Telfa wound dressing.

### Example 2

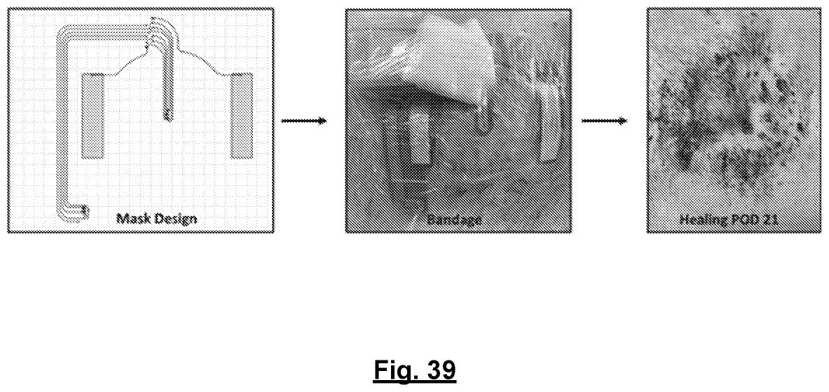

This Example describes development and testing of a multi-material, flexible substrate (FIG. 39) (referred to below as “exciflex”) that integrated AFTIDerm and an elastomeric nanocomposite (which is referred to in this Example as “Flexatrode” and disclosed in PCT App. No. PCT/US2021/26571, filed Apr. 9, 2021, entitled “Flexible nonmetallic electrode”) and other bandage components, such as an electronics module. A flexible substrate, comprised of copper-clad polyimide, was etched using microfabrication protocols to create copper electrodes (4 cm length×1 cm width) and copper traces (75-300 μm pitch), the latter to facilitate the transmission of the electrical stimulation (ES, current amplitude 16 mA, pulse width 100 μs, interpulse interval 50 ms, current density 5.3 μA/cm²) to the skin. A printed circuit board (PCB), integrated with various surface mount components, was connected to the substrate using soldered connections and to a lithium-ion polymer battery (400 mAh, 5 mm×36.9 mm×26.5 mm). To prevent biofouling on the copper electrodes, Flexatrode was integrated via a conductive double-sided biocompatible adhesive to serve as an interface between the copper electrodes and skin. A silicone acrylate tape was secured to the skin-facing side of the substrate to enable adhesion of the device to the skin. AFTIDerm (100 μm thickness) comprised of PVA and glycerol was synthesized and integrated with the flexible Cu—PI substrate to complete the device to serve as an occlusive wound dressing. The diameter of the wound dressings, which ranged from 4-6 cm, were tailored to match the re-epithelization status of the wound. The efficacy of the devices was tested in a porcine chronic wound model (n=10 wounds per treatment group, 6 cm diameter wound, 2.5-3 mm deep). Six bilateral excision wounds (2 standard of care; HP Tegaderm and Telfa, 2 inactive devices, and 2 ES treatment) were created on each pig, with each animal serving as its own control. Ischemia was created by placing and suturing silicone flanges in the wounds for 14 days following surgery with bandage changes occurring at pre-determined time points. The procedure was considered terminal one week after re-epithelization of the active wounds. On average, pH measurements showed a decrease in the active wounds from 7.5 to 6.8 over 35 days, suggestive of improved healing. This data represents one of the first longitudinal data sets involving multiple large animals studying the effect of pH on wound healing. In short, this Example provides initial subjective and objective data to which verified the efficacy of the exciflex device for wound healing when on the skin for a one-week period.

Substrate Fabrication, Integration, and Benchtop Testing

Substrate Fabrication

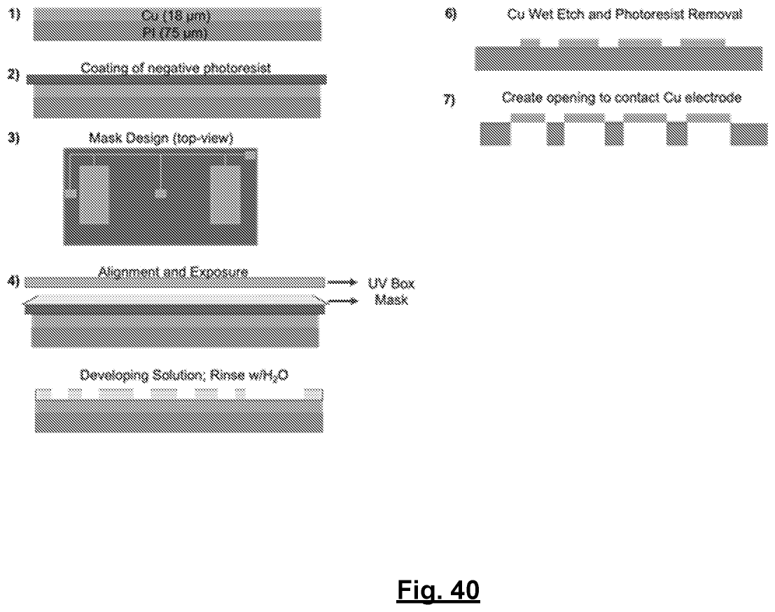

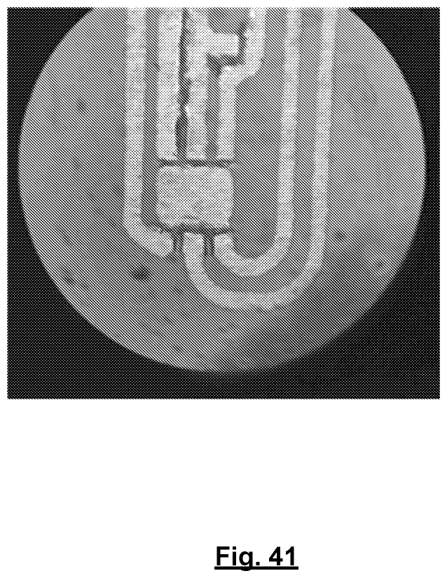

Substrate prototypes were fabricated leveraging conventional integrated circuit fabrication processes (FIG. 40). Briefly, a mask was designed using AutoCad to pattern the electrodes, contact pads, and traces. A negative photoresist was applied and laminated onto the copper-side of the copper-clad polyimide (Cu—PI) substrate. Exposing the mask to the substrate via UV light (365 nm) transferred the layout onto the (Cu—PI) substrate. The substrate was then placed in developer's solution, comprised of sodium bicarbonate and water, to remove the unreacted photoresist. Subsequently, the substrate was etched in a sodium persulfate solution in water for one hour. The remaining photoresist was removed, substrate cleaned with IPA, and used for testing or integration with the bandage. The traces were then visually inspected to assess fidelity post fabrication (FIG. 41).

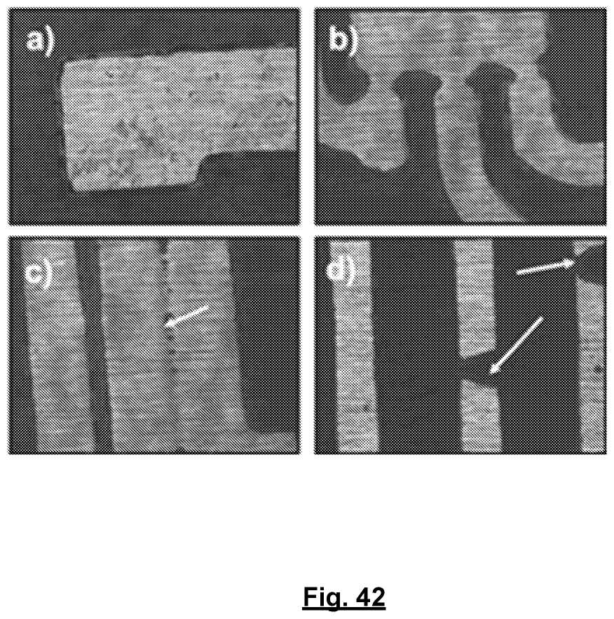

Parameters such as photoresist type (dry versus gel), exposure time, etchant concentration, development time (in the case of a gel photoresist), pre- and post-bake time (in the case of a gel photoresist, FIGS. 42(a)-(d)), and trace dimensions were optimized during the fabrication process (Table 6).

TABLE 6
Summary of process development efforts for substrate fabrication
(WT: Wound temperature sensor; AT: Ambient temperature sensor)

| | | | | Rationale for Change |
|---|---|---|---|---|
| Dimensions | Mask 1.0 | Mask 2.0a | Mask 2.0b | from Mask 1.0 to 2.0 |
| Substrate | 10.5 cm × 10.5 | 10.5 cm × 10.5 | 10.5 cm × 10.5 | N/A |
| dimensions | cm | cm | cm | |
| Trace pitch | ~100 μm | 300 μm | 300 μm | Lower trace impedance |
| Sensors | | | | and prevent trace fracture |
| Trace pitch | ~100 μm | 800 μm | 800 μm | Lower trace impedance |
| Electrodes | | | | and prevent trace fracture |
| Electrode | 4 cm length × 1 | 4 cm length × 1 | 4 cm length × 1 | N/A |
| dimensions | cm width | cm width | cm width | |
| Trace pitch | WT: 350 μm | WT: 350 μm | WT:350 μm | Lower trace impedance |
| | AT: 500 μm | AT: 750 μm | AT: 750 μm | and prevent trace fracture |
| Feature Size | <100 μm | <100 μm | <100 μm | N/A |
| | (contact pad SMT | (contact pad SMT | (contact pad | |
| | capacitor) | capacitor) | SMT capacitor) | |
| Photoresist | Negative dry film | Negative dry film | AZ ® nLOF ™ | Use of a dry film |
| | resist | resist | 2035 | photoresist precluded the |
| | | | | need for a pre- and post- |
| | | | | bake step |
| Developer's | Sodium | Sodium | TMAH | Sodium bicarbonate |
| Solution | Bicarbonate | Bicarbonate | | precluded the need for a |
| | | | | pre- and post-bake step |
| Etchant | Sodium | Sodium | Sodium | N/A |
| | Persulfate | Persulfate | Persulfate | |

The substrate dimensions were kept the same at 10.5 cm×10.5 cm for a 6 cm wound. The trace pitch for the temperature sensors was increased from 100 μm to 300 μm to minimize impedance and prevent trace fracture during fabrication. As shown in the equations below, increasing the trace width increases the cross-sectional area and would decrease the overall impedance.

$$R = ρ \frac{L}{A}$$

$$R = ρ \frac{L}{w * t}$$

Electrode dimensions were set at a length of 4 cm and width of 1 cm to factor in wound re-epithelization. Processing parameters involving the use of a gel and dry photoresist are summarized (Table 7).

TABLE 7
Summary of processing parameters when using the
gel-based photoresist and dry film photoresist

| | Pre-bake | Exposure | Post-bake | Develop | Etch |
|---|---|---|---|---|---|
| Photoresist | Time | Time | time | Time | Time |
| Gel | 110° C., | 80 mJ, | ~110° C., | 25 sec | 2 hours |
| | 60 sec | 300 sec | 120 sec | | |
| Dry Film | N/A | 80 mJ, | N/A | Minutes | 2 hours |
| | | 60 sec | | | |

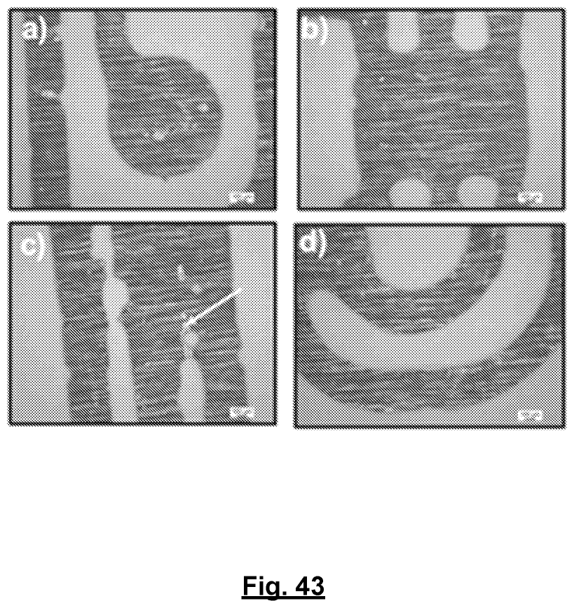

A negative dry film photoresist was used in lieu of the gel-based photoresist with the goal of achieving the minimum viable feature size of less than 100 μm for the bandage (FIGS. 43(a)-(d)).

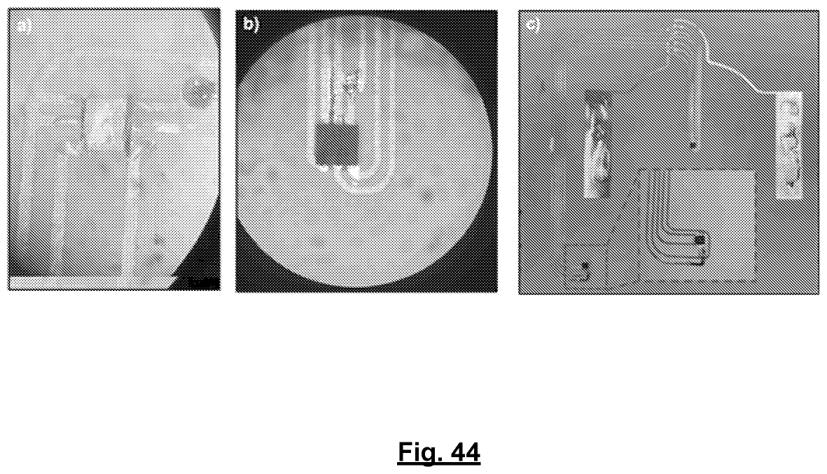

Substrates were visually inspected following fabrication and surface mount components (e.g., temperature sensors and capacitors) were integrated using a silver epoxy (FIGS. 44(a)-(c)). Development of an initial prototype then led to benchtop testing of the bandage components prior to large-scale manufacturing efforts of the substrates required for pre-clinical assessment.

Substrate Integration and Testing

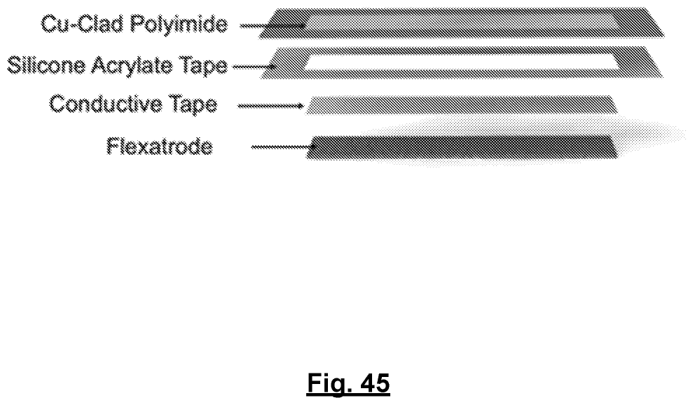

Following fabrication of the substrates and integration of the surface mount components, Flexatrodes were integrated onto the bandage. The through thickness impedance of the substrates and Flexatrode was evaluated. Flexatrode were adhered to the Cu—PI substrate via the use of a double-sided conductive tape (FIG. 45).

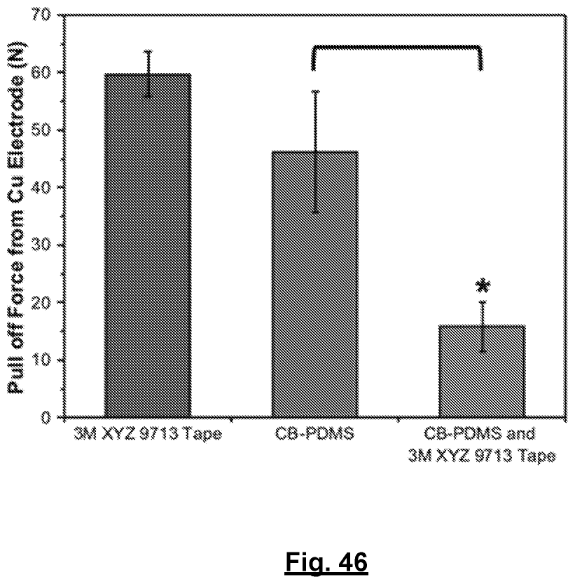

Adhesion (vertical pull-off force) between the interfaces was also evaluated (FIG. 46). 3M XYZ 9713 conductive tape was applied on the copper electrode (blue). Flexatrode slurry (CB-PDMS) was cured directly on the copper electrode (purple). Additionally, Flexatrode was also adhered with the 3M 9713 Conductive tape to the copper electrode (green). The 3M XYZ 9713 conductive tape had the strongest adhesion to the copper electrode (˜60 N), followed by adhesion of Flexatrode when directly cured on the copper electrode (˜45 N), followed by the adhesion of Flexatrode and the 3M XYZ 9713 conductive tape directly on the copper electrode (˜15 N). While adhesion data alone would suggest that the use of the conductive tape would suffice as a conductive interface to the copper (to prevent biofouling), the presence of the tape with water (or exudate as is the case with wounds) would cause it to shrink and delaminate from the copper electrode thereby mitigating its standalone utility. For that reason, the conductive tape served as an interface with Flexatrode to help adhere the two together. Thus, in terms of engineering design, the Flexatrode provided the necessary conductivity to facilitate the delivery of the electrical stimulation while preventing adsorption of proteins and biologics found in the exudate onto the copper electrode.

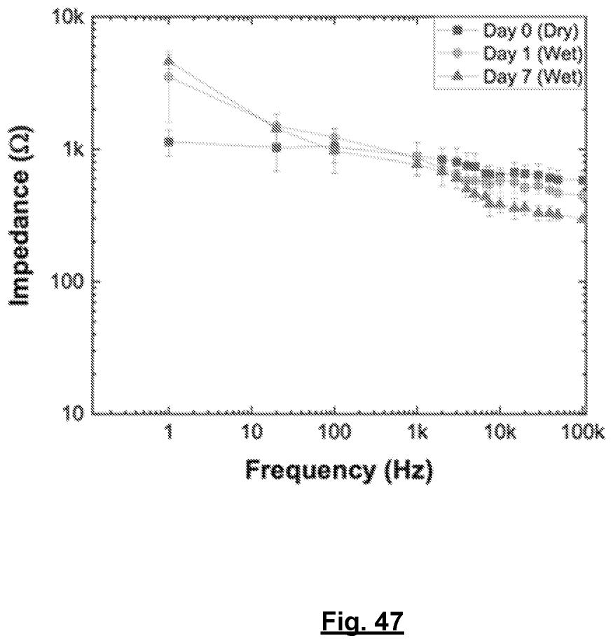

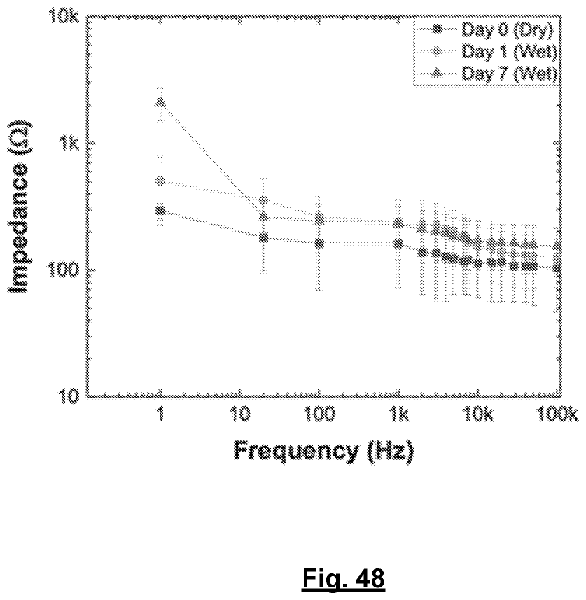

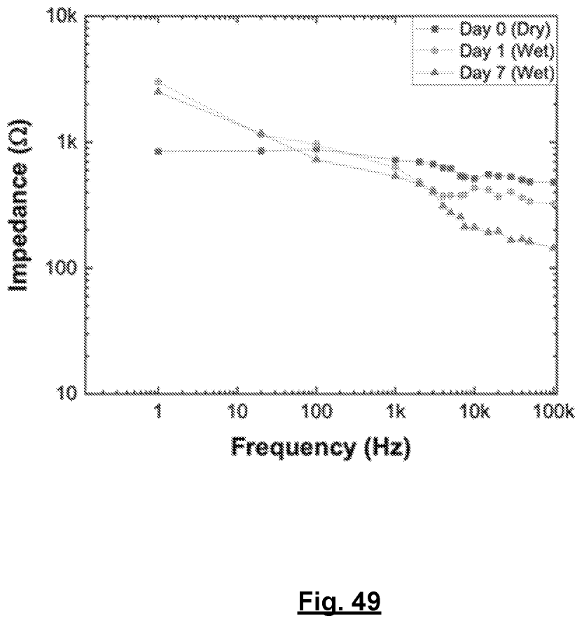

Through thickness impedances when adhered to a copper electrode (Flexatrode and Conductive tape, FIG. 47), Flexatrode (FIG. 48), and conductive tape (FIG. 49) was determined over 7 days. Examples of this comparison are provided at 1 KHz and 10 KHz (Table 8 and Table 9).

TABLE 8
Through-thickness impedance over a one-week
period at 1 KHz (data presented as n =
3; mean ± std. deviation where provided)

| | 1 KHz | | |
|---|---|---|---|
| Through Thickness | Day 0 | Day 1 | Day 7 |
| Flexatrode and Tape (Ω) | 882.6 ± 245 | 867.7 ± 63.7 | 767 ± 134 |
| Flexatrode (Ω) | 161.3 ± 87.6 | 238 ± 117 | 230 ± 88.6 |
| Conductive Tape (Avg Ω) | 721.3 | 629.7 | 537 |

TABLE 9
Through-thickness impedance over a one-week
period at 10 KHz (data presented as n =
3; mean ± std. deviation where provided)

| | 10 KHz | | |
|---|---|---|---|
| Through Thickness | Day 0 | Day 1 | Day 7 |
| Flexatrode and Tape (Ω) | 626 ± 95.6 | 585.6 ± 118 | 382 ± 51 |
| Flexatrode (Ω) | 113 ± 52.3 | 153 ± 68 | 172 ± 70 |
| Conductive Tape (Ω) | 513 | 432 | 210.3 |

At 1 kHz, Flexatrode accounted for 18.3-29.9% of the overall through-thickness impedance. At 10 kHz, Flexatrode accounted for 18.1-45% of the overall through thickness impedance. Results demonstrated the long-term electrical stability of the Flexatrode in dry and hydrated environments.

Exciflex 1.0-3.0 Substrate Iterations

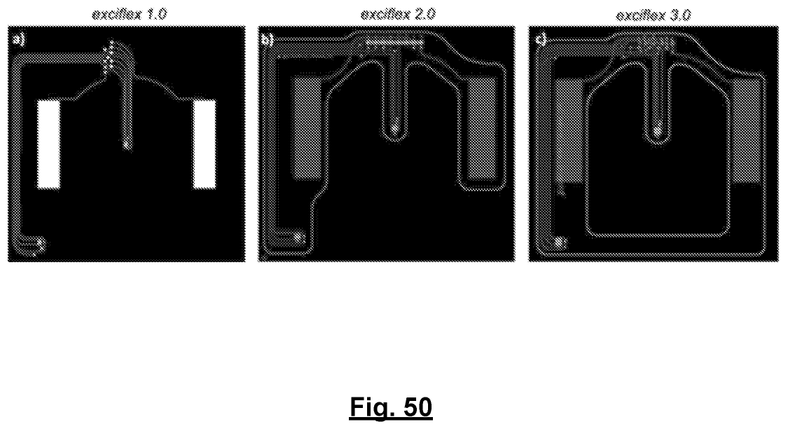

The improvements in design and substrate layout for the exciflex bandage were made based on observations made during the pre-clinical procedures (FIGS. 50(a)-(c)). Major development efforts from exciflex 1.0 to exciflex 3.0 are summarized below:
exciflex 1.0 enabled the integration of a rigid electronics module along with the flexible substrate and enabled viewing of the wound;
exciflex 2.0 was fabricated in 3 sizes to factor in wound re-epithelization (6 cm, 4 cm, and 2 cm). The contact pads to integrate the rigid electronics module was rotated 90° to the top of the bandage such that the electronics module and battery lie on the paraspinal region and not on the soft-tissue of the pig. AFTIDerm was integrated with the bandage such that the bandage and wound dressing were placed as one piece; and
exciflex 3.0 further improved upon the location of the contact pads by staggering them on top of the bandage. This helped ease stress of the soldered contacts to the electronics module. Furthermore, AFTIDerm integration with the bandage was improved by maximizing the surface area of adhesion to the substrate. Lastly, the adhesion of the bandage to the skin was improved by increasing the surface area of adhesive to the skin by having the bandage serve as one connected piece.

Exciflex 1.0

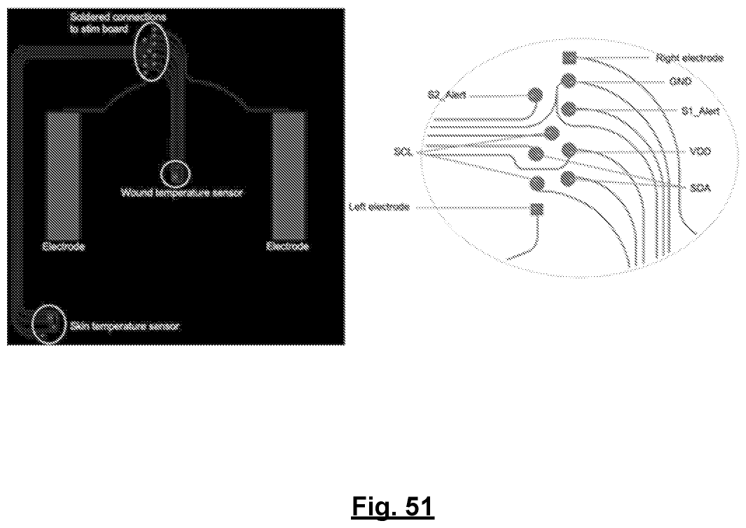

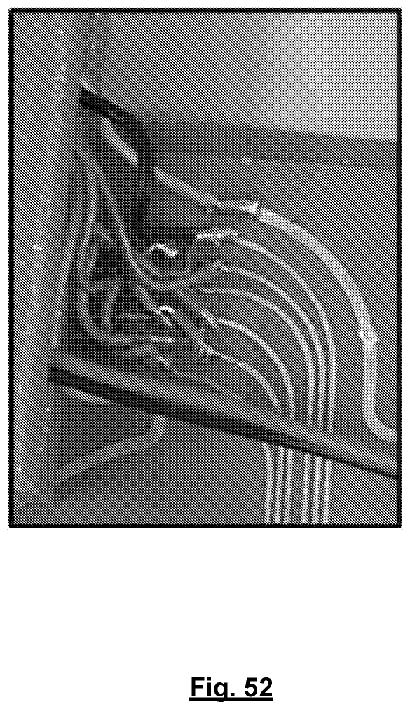

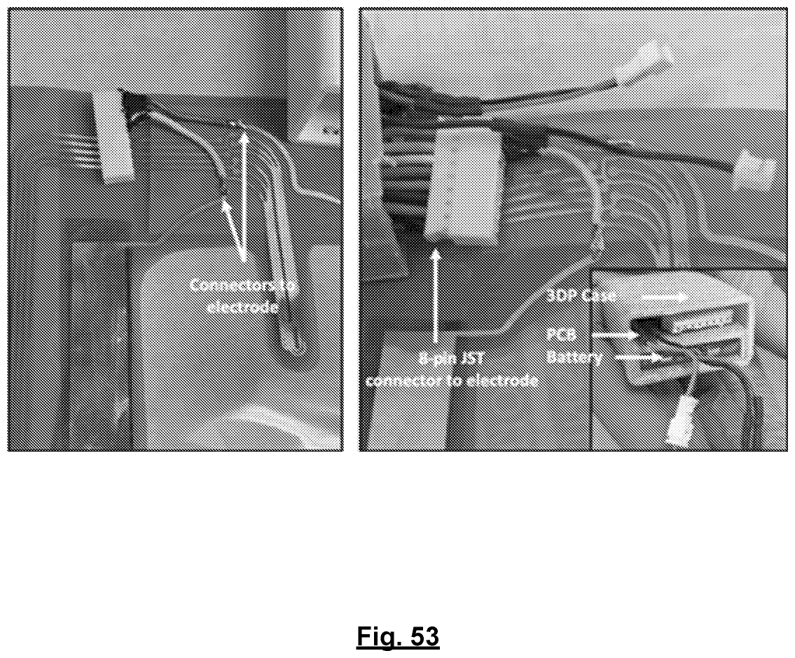

Exciflex 1.0 enabled the integration of a rigid electronics module (e.g., printed circuit board, PCB) along with the flexible microfabricated substrate. The substrate consisted of two copper electrodes (length 4 cm×width 1 cm), two temperature sensors, traces (pitch 300 μm), and contact pads to facilitate the integration (FIG. 51). Initial integration of the rigid electronics module to the substrate involved soldering wires to the contact pads and to the PCB (FIG. 52). During initial bandage changes during the pre-clinical studies, this approach proved to be cumbersome and not clinically viable as the team was unable to solder and desolder wires from the contact pads when with the pig. To circumvent this hurdle, an 8-pin JST connector with the necessary adapter was utilized instead (FIG. 53). Wires (36 gauge) were soldered directly onto the contact pads of the substrate and connected to the 8-pin JST adapter. Wires were then soldered to the rigid PCB and connected to the lithium-ion polymer battery to enable integration of the battery to the JST connector and from the electronics module to the battery. This modification enabled efficient attachment and detachment of the PCB and board during subsequent bandage changes.

Exciflex 1.0 leveraged advancements in materials fabrication by incorporating Flexatrode (elastomeric nanocomposite) and AFTIDerm in the bandage. While exciflex 1.0 demonstrated its efficacy in delivering the electrotherapy to the wound, the scalability of the bandage coupled with the need to achieve feature sizes <100 um for the temperature sensors and capacitors proved to be challenging for long-term pre-clinical use. Thus, for the next iteration in substrates, an external manufacturer (PCBWay) was employed to achieve the necessary feature sizes for the bandage and to scale this validated substrate design. Furthermore, the need to integrate AFTIDerm with the bandage (rather than applying it to the wound separately followed by application of the exciflex substrate) will expedite the bandage change process. Furthermore, integration of AFTIDerm with the bandage will enable the removal of the silicone adhesive which was currently used to adhere AFTIDerm to the skin. The silicone adhesive utilized was Ecoflex-35, a hydrophobic biocompatible gel. Alleviating the application of this gel (due to the silicone acrylate adhesives on the exciflex substrate) would leverage the hydrophilicity of the PVA thus maximizing the absorption capabilities of AFTIDerm. Lastly, the orientation of the contact pads resulted in the battery and PCB being situated over soft-tissue which resulted in pressure-induced injuries at certain time points. The orientation of these connections was rotated 90° clockwise such that the PCB and battery lie over the paraspinal region. Furthermore, exciflex 1.0 was fabricated to one size (8.7 cm×9 cm), not factoring in changes in wound healing status.

Exciflex 2.0

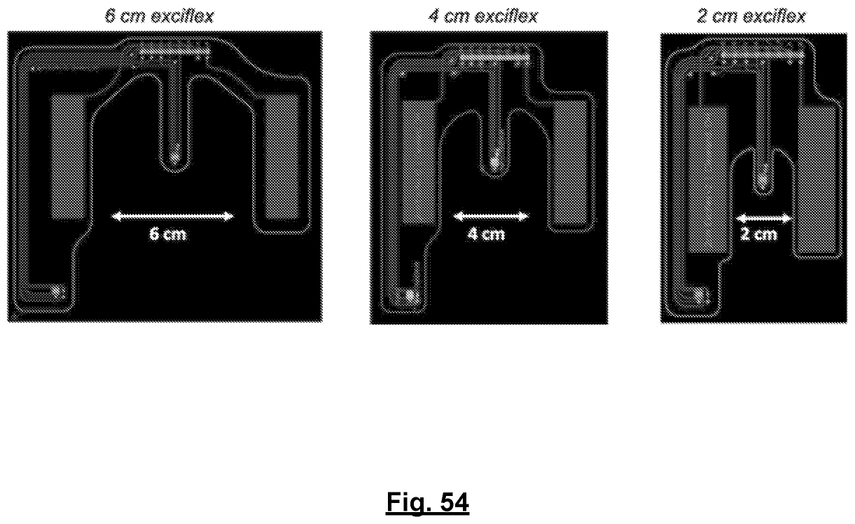

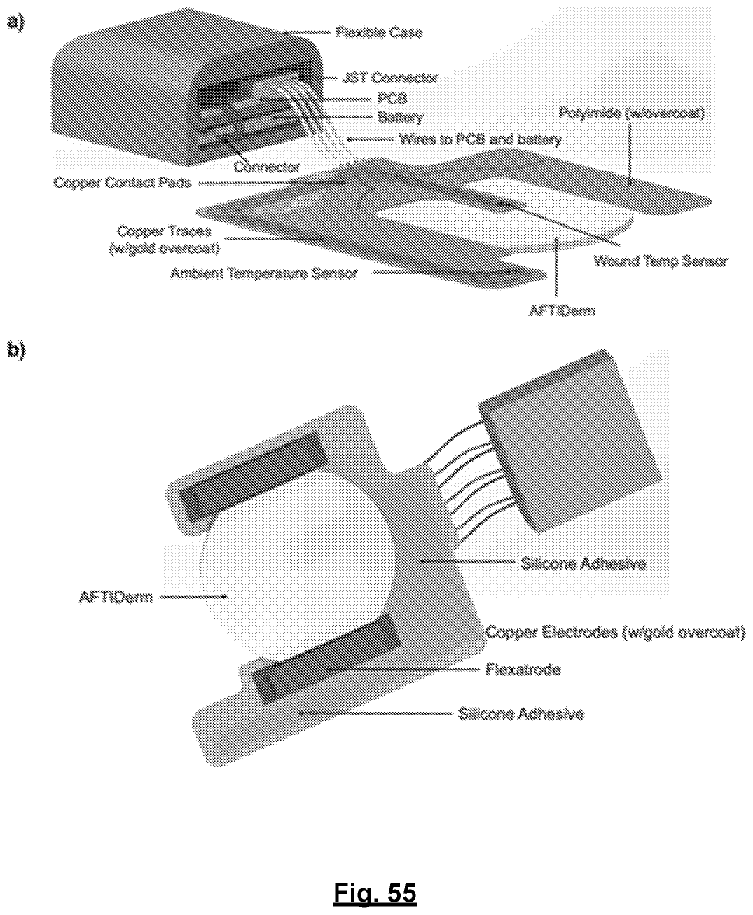

Exciflex 2.0 was designed to meet the wound healing timeframe by tailoring the size of the bandages to that observed during wound reepithelization (FIG. 54). Three bandage sizes were created: 6 cm (8.7 cm×9 cm), 4 cm (8.7 cm×7 cm), and 2 cm (8.7 cm×5 cm) (FIG. 55(a)-(b)).

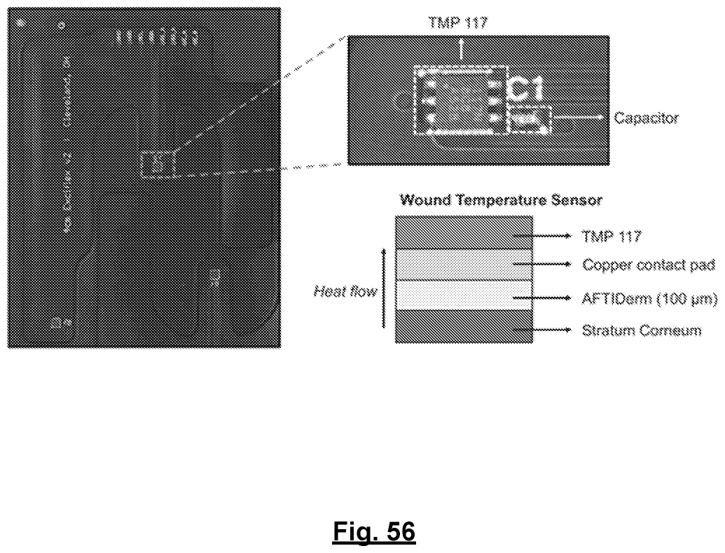

Exciflex 2.0 demonstrated the necessary feature sizes <100 um to integrate the temperature sensors and capacitors (FIG. 56). The TMP-117 (2 mm×2 mm) is a high-precision digital temperature sensor designed to meet ASTM E1112 and ISO 80601 requirements for electronic patient thermometers. The TMP-117 provides a 16-bit temperature result with a resolution of 0.0078° C. and an accuracy of up to ±0.1° C. (clinical standard) across the temperature range of −20° C. to 50° C. with no calibration. The TMP-117 operates from 1.7 V to 5.5 V and consumes ˜3.5 μA. The low power consumption of the TMP-117 minimizes the impact of self-heating on measurement accuracy. TMP-117 operates as a pyroelectric sensor that converts the heat radiated from the skin to a voltage output to the board and a temperature value on a corresponding application platform.

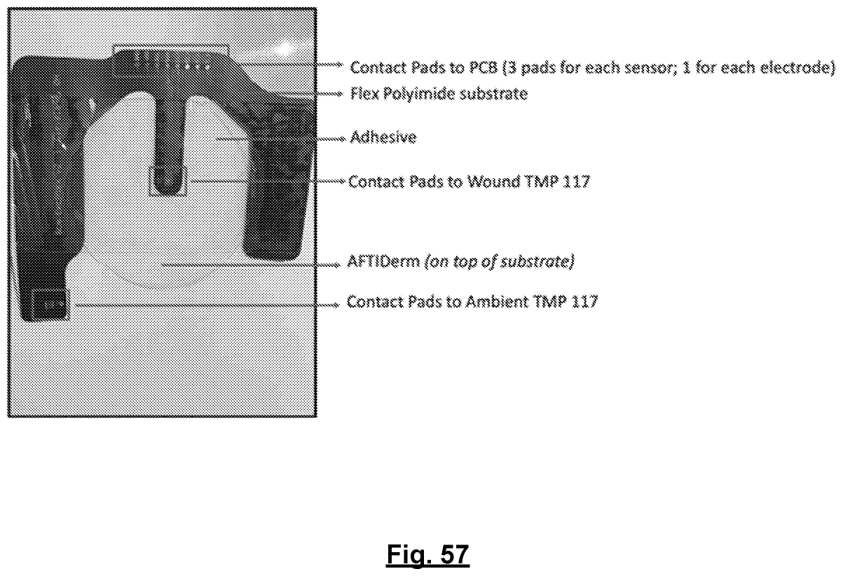

Furthermore, exciflex 2.0 integrated AFTIDerm with the bandage as one piece towards creating a fully integrated bandage with the substrate, Flexatrode, and AFTIDerm components. AFTIDerm was integrated to the substrate via the use of a silicone acrylate adhesive (3M 2477P) (FIG. 57). Lastly, the orientation of the contact pads resulted in the battery and PCB being situated over the paraspinal region which when housed in an elastomeric housing, drastically reduced pressure-induced injuries on the skin.

Exciflex 3.0

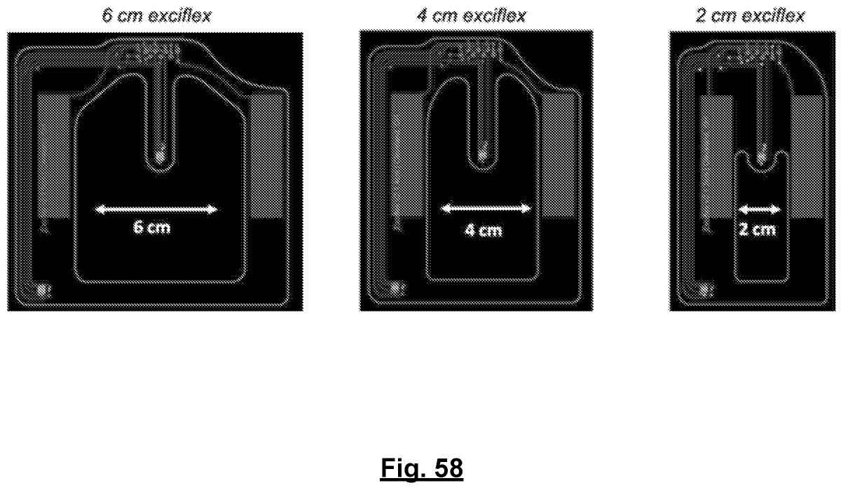

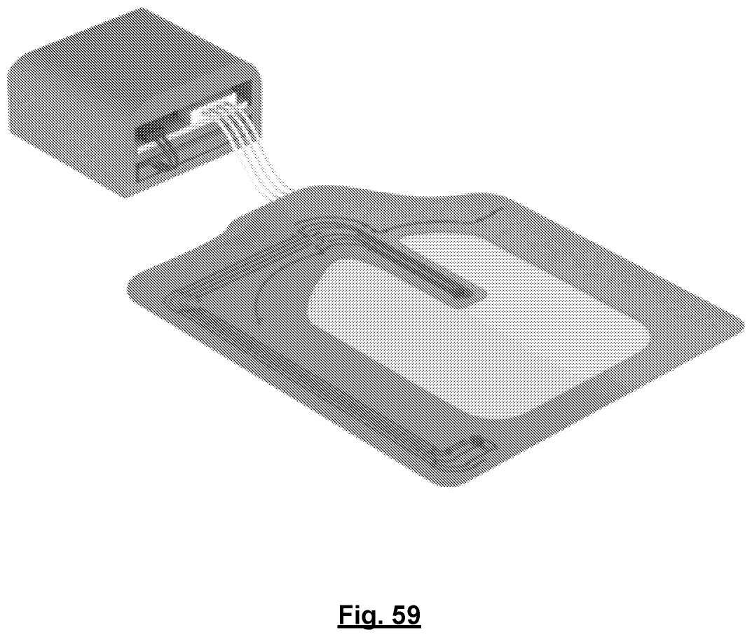

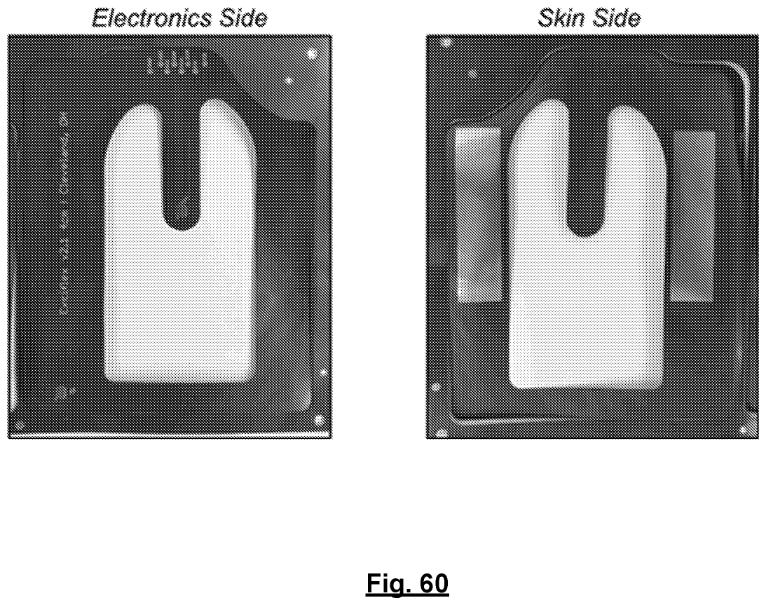

The major modifications for exciflex 3.0 involved staggering the location of the connectors on the bandage to ease with integration of the PCB and substrate (FIG. 58). Furthermore, unlike exciflex 1.0 and 2.0 where the bandage was essentially two halves, exciflex 3.0 substrate was connected to maximize the surface area of the silicone acrylate adhesive to the skin and further assist in the integration of the AFTIDerm to the substrate (FIGS. 59-60).

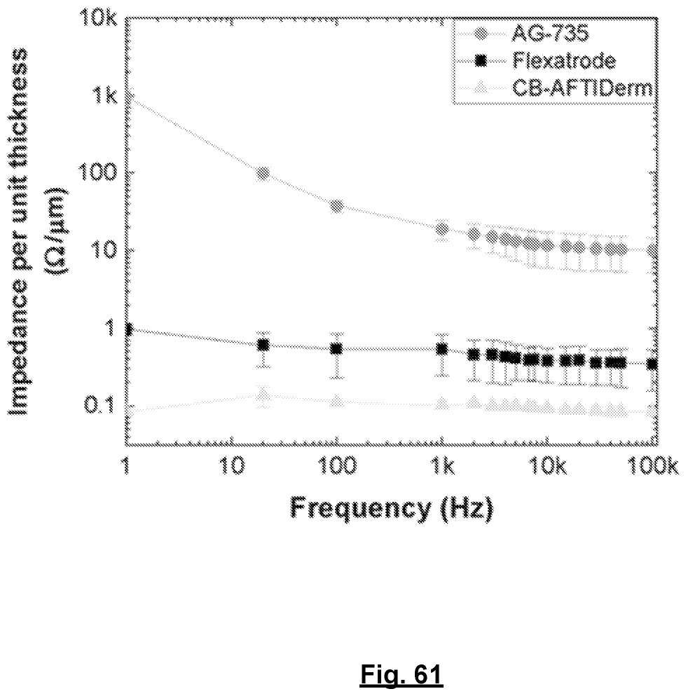

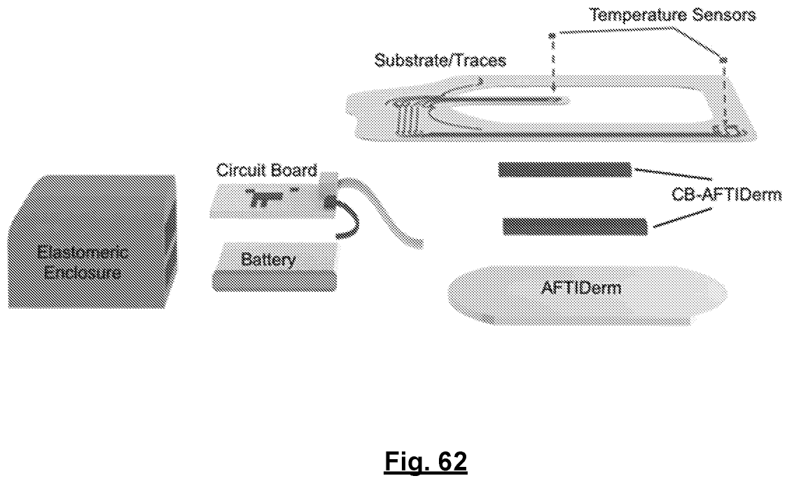

Flexatrode (length: 4 cm, width 1 cm, thickness 300 μm) had through thickness impedance of ˜1000Ω, indicative of a resistance of 3.3 Ω/μm and an average resistivity of 7.5 Ω-cm. Similarly, CB-AFTIDerm (length: 4 cm, width 1 cm, thickness 100 μm) has a through thickness impedance of ˜10Ω, indicating a resistance of 0.1 Ω/μm and an average resistivity of 0.025 Ω-cm (FIG. 61). Thus, material developments and innovations facilitated the improvement in flexible electrode technology to enhance delivery of the electroceutical therapy from the electronics module by 300-fold through the traces and to the wound microenvironment when comparing resistivities. While Flexatrode was used as the electrode for the pre-clinical studies, use of CB-AFTIDerm electrodes is additionally or alternatively contemplated (FIG. 62).

Pre-Clinical Study

Following integration and benchtop assessment of the exciflex bandage, pre-clinical evaluation of the effects of selected clinically relevant treatment paradigms was performed using our Yorkshire pig large wound model modified to create ischemic wounds.

Surgery and Wound Creation

Each animal had six wounds. Two wounds were covered with a Tegaderm dressing (negative control), two were covered with an exciflex bandage that monitored healing but did not deliver electrotherapy (positive control, herein referred to as the inactive device), and two were covered with an exciflex bandage that monitored healing and delivered electrotherapy (intervention, herein referred to as the active device). Each animal was used as its own control in order to maximize efficiency. Serial evaluations of the same wounds were carried out in a time series study to determine temporal variations in the response of wound healing outcomes to ES. These wounds are of a clinically relevant size and were evaluated at multiple timepoints, thus all measurement techniques used were minimally invasive.

Six full-thickness excisional wounds were created bilaterally over the paraspinal region in each animal. Intra-operative anesthesia was maintained by isoflurane. The back of the animal will be clipped and then wiped liberally with 4% chlorohexidine. A template was used to mark the locations of wounds to be created. Full-thickness wounds (6 cm diameter) were excised bilaterally over the paraspinal/flank region at a distance of 4 cm.

In order to create an ischemic wound, a sterile double-flanged silicon block was placed into each wound. The flanges were 9 cm in diameter and 0.5 cm high. The central core of the wound insert block was 6 cm in diameter and 1 cm high. Each wound was covered with a Tegaderm dressing. The animals were wrapped in an elastic bandage (VetRap® 3M Health Care, St Paul, MN) to prevent animals interfering with the system. The pigs were covered with a protective body jacket (Goat Tube®, Sullivan Supplies, Houston, TX) to prevent environmental contamination. The animals were awakened from general anesthesia, given post-operative analgesia, and placed in single-occupancy pens. They were maintained with standard laboratory feed and water ad libitum.

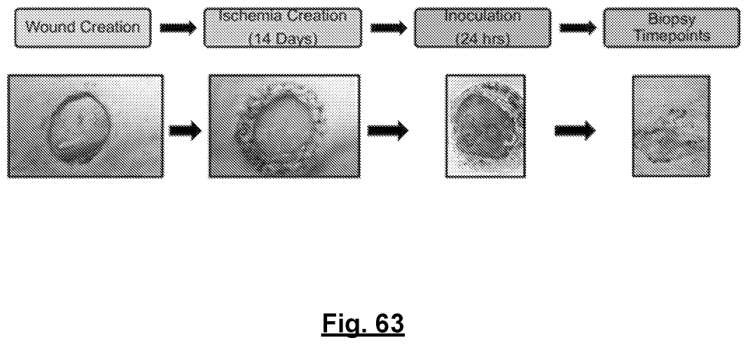

Wound insert blocks were left in situ for 14 days. At this time, the blocks were removed one at a time. Each wound was evaluated following the assessment protocol established in our previous work together with baseline assessment of bacterial contamination status. Following the 14-day period, the wound plugs were removed and all wounds were inoculated with 0.5 McFarland solution of GFP labeled P. aeruginosa as used in our preliminary work. Inoculation with P. aeruginosa facilitated the formation of a biofilm layer on the wound (FIG. 63).

Electroceutical Treatment and Monitoring

During each experiment, electrical stimulation (ES) was applied to two wounds using exciflex applying a 10% duty cycle. The same stimulation paradigm was applied in both actively treated wounds. Two control untreated wounds were covered with a non-active exciflex (inactive device). Two wounds acted as control treated wounds with a standard of care wound dressing (Telfa) together with Tegaderm™. It was not known whether different regions of the pig's back will respond differently to ES. Exciflex was therefore activated in a random pattern within each group such that two devices were delivering active ES and two devices were not. For example, the exciflex devices were activated so that on one flank the rostral wound received ES, the central wound was covered with an inactive exciflex, and the caudal wound was a positive control, covered with a standard of care wound dressing plus Tegaderm™. On the other flank, the central wound was not stimulated receive ES, the rostral wound covered with a standard of care wound dressing plus Tegaderm™ and the caudal received ES. ES was delivered for up to 7 week or until all wounds are healed for 7 days. Wounds were assessed at time-points relevant to the normal course of wound healing, specifically biopsy timepoint days (BTD) 1, 3, 7, 10, 14, 18, 21, 28, 35, and 42. Aseptic technique was used at each assessment. At each time-point, the pig was sedated using 6-10 mg/kg IM Telazol. The outer protective layers were removed to expose the exciflex devices and standard of care dressings covering the wounds. The coverings were removed from each wound in turn and wound status monitored as described next.

At each dressing change, the active exciflex delivering ES was turned off and the bandage was removed from the wound. Macroscopic assessments included digital imaging to quantify wound size, vascularization, and monitor signs for infection, swabbing to determine infection status, and surface pH measurements. Surface pH was measured at the wound center and/or margin (depending on wound re-epithelization status and location of biopsies) using a portable skin pH meter (Hanna Instruments, Ann Arbor, MI). Wound biopsies provided tissue for histology and real-time PCR assessment of wound healing markers. Wound tissue was harvested per the following protocol. Specifically, 4 mm biopsies were harvested from the wound center and margin at each timepoint. Tissue sections were divided and stored for further analysis. Specifically, a double swab packaging was used to obtain bacteria for culture (BBL™ CultureSwab™, Franklin Lakes, NJ). The double culture swab was applied with a gentle pressure to the wound's surface. The entire wound was swabbed from top to bottom using a back-and-forth motion. The swab was then rotated 180 degrees and the entire wound swabbed again using the same technique. In addition, bioburden was evaluated by collecting three further swabs from the wound bed as described by Sprockett et al., Wound Repair Regen. Off. Publ. Wound Heal. Soc. Eur. Tissue Repair Soc. 23, 765-771 (2015). The wound bed was wiped with sterile gauze moistened with normal saline. The wound bed was swabbed using Catch-All Collection Swabs (Epicenter) soaked in sterile 0.1% Tween 20 in PBS collection buffer. The swabs were rolled over a 1 cm² areas at the wound center and wound edge for 10 s, using sufficient pressure to extract wound fluid. All swabs were stored at −80° C. until further analysis. This concluded the data analysis process.

For wounds covered with exciflex, the power/control module was transferred to a new sterile flexible substrate and exciflex reapplied to the wound. Once the wound was covered, exciflex was activated or left inactive as appropriate. The next wound was then uncovered and wound status monitored in the same manner. Wounds with a standard of care hydrogel dressing plus Tegaderm™ received a fresh sterile dressing of the same type.

All pigs were sacrificed at the end of the stimulation period in order to harvest tissue at the wound site. Euthanasia was carried out by administration of 6-10 mg/kg IM Telazol for sedation and 100 mg/kg IV Euthasol. This method followed the recommendations of the AVMA Panel on Euthanasia and has been approved for use by our group in a pig study previously active at CWRU.

Results

Bandages were changed at BTD 1, 3, 7, 10, 14, 17, 21, 28, 35, and 42. Each animal served as its own control with 2 wounds with the exciflex bandage receiving electrical stimulation, 2 wounds with the exciflex bandage not receiving electrical stimulation, and 2 wounds receiving standard of care. Bright field imaging, infrared thermography, and wound pH is presented herein demonstrating the efficacy of the exciflex bandage for chronic wound healing.

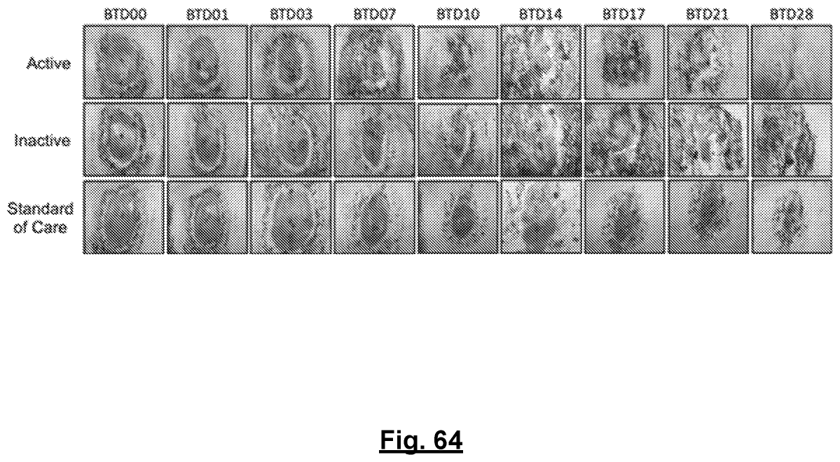

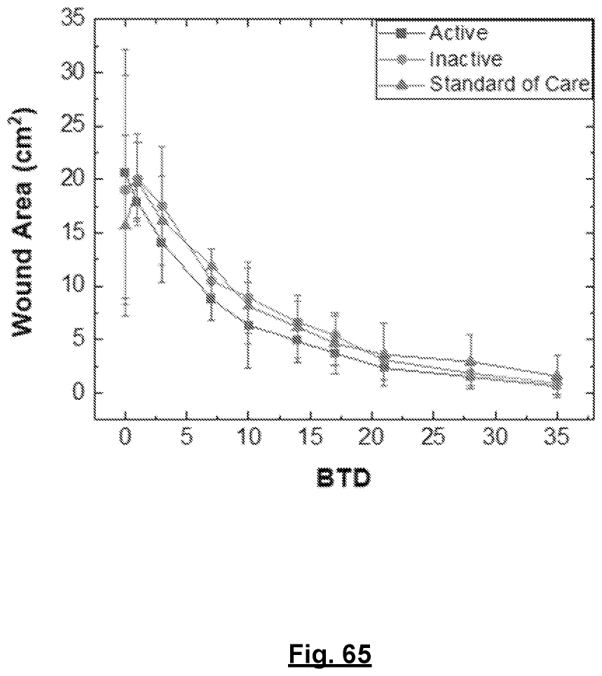

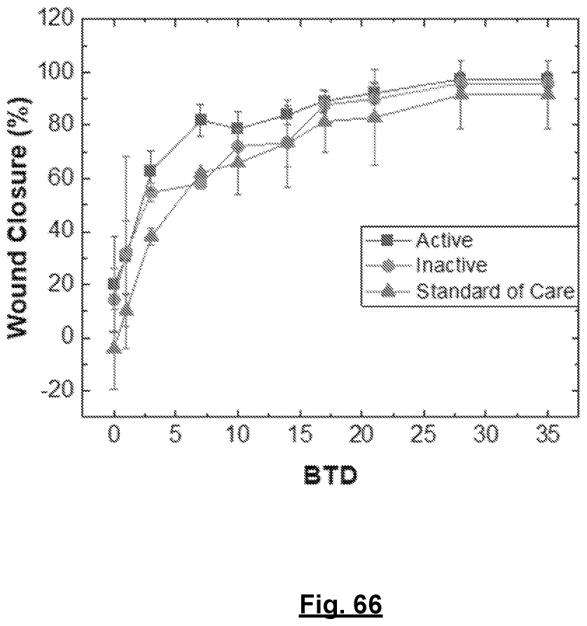

Bright field imaging shows the re-epithelization of the wounds over 35 days (FIG. 64). In this study (focusing on ischemic wounds), the inventors found that electrotherapy treated chronic wounds 81.9% smaller than baseline at day 10. Wounds that received an inactive device (exciflex device without any electrical stimulation), were 58.1% smaller than baseline and wounds that received standard of care treatment were 62.2% smaller than baseline (FIGS. 65-66).

Change in Wound Closure (%) Over Time

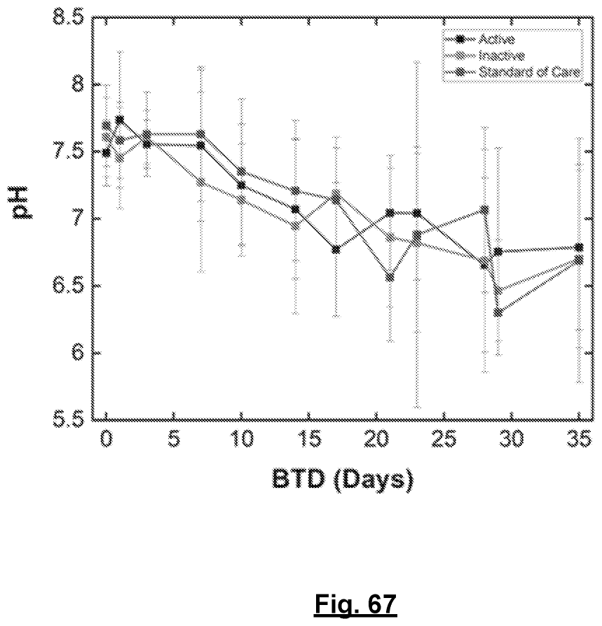

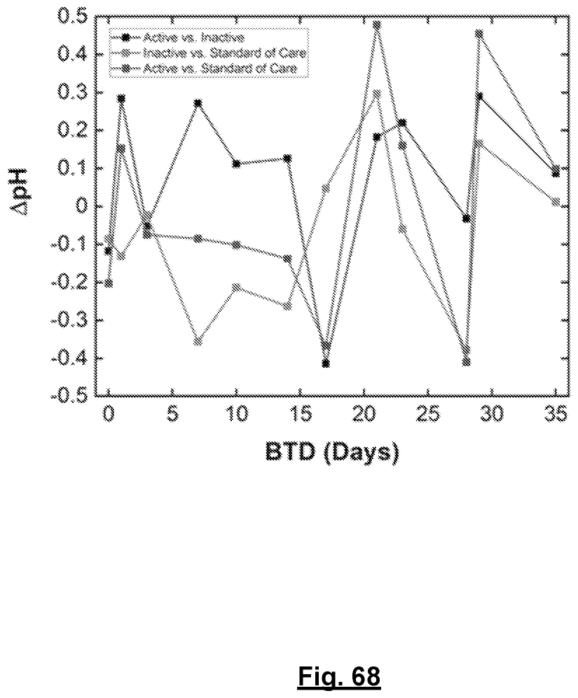

In this study, pH was measured using a glass membrane probe with measurements taken prior to each biopsy. Wound healing pH decreased over a 35-day span in the ES treated wounds from 7.5 to 6.8, from 7.6 to 6.7 in wounds receiving an inactive device, and from 7.7 to 6.7 in wounds that received a standard of care dressing (FIG. 67). While each treatment group observed a decrease in pH, the results confirmed that the delivery of ES to the wound microenvironment from a closed-loop system did not have a deleterious impact on wound healing and in fact aided in healing analogous to wound dressings used in the clinic (e.g., Telfa and HP Tegaderm). The differences among the various treatment groups was within a pH range of 1 thereby confirming that the delivery of ES and the presence of the exciflex bandage did not adversely affect the wound (FIGS. 67-68).

Difference in pH Between Treatment Groups Over Time

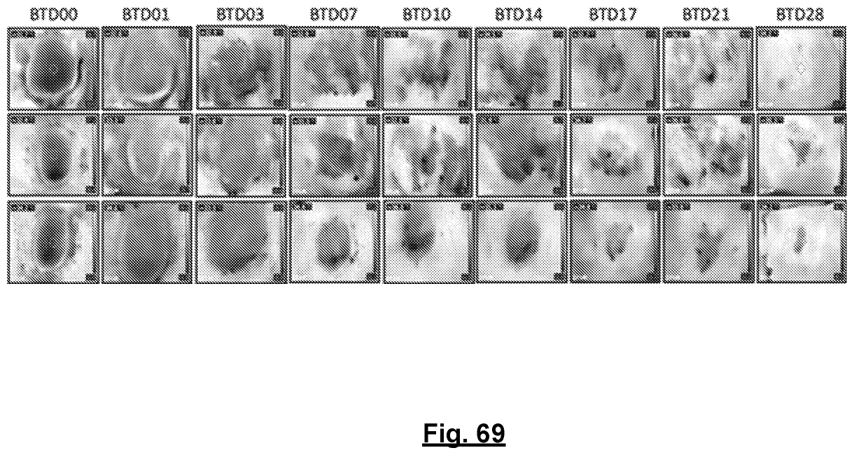

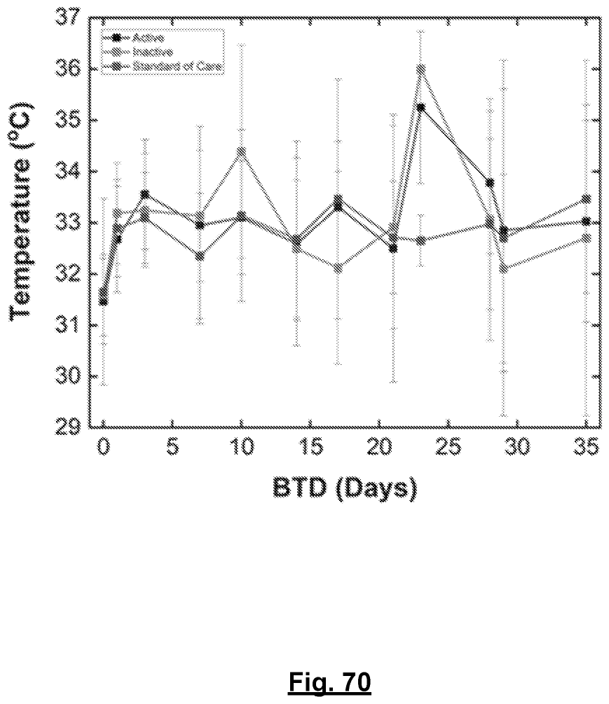

Infrared thermography was performed during the bandage change procedure to provide qualitative (FIG. 69) and quantitative (FIG. 70) insight regarding ischemia, inflammation, and infection of the wound and surrounding microenvironment. In the case of chronic wounds, tissue cooling in and around the wound microenvironment leads to an increased risk of infection because it can cause vasoconstriction and increase hemoglobin's need for oxygen. This results in decreased oxygen available for neutrophils to fight infection. Furthermore, neutrophil, fibroblast, and epithelial cell activity declines as temperature drops. Hypothermia also inhibits platelet activation, oxidative killing by neutrophils, and a reduction in wound strength as collagen deposition declines. Dressing changes, along with wound cleansing, can decrease wound temperature and cellular activity. Thus, decreasing the frequency of dressing changes is beneficial towards improving healing outcomes. Towards addressing this clinical need, the exciflex bandage incorporates the AFTIDerm dressing which can be kept on the skin for up to one week without the need for repetitive changes. Overall, there was no significant difference in temperature records between the three treatment groups. Over a 35-day period, there was a 1.6° C. difference in the wounds that received ES, 1.1° C. difference in the wounds that received an inactive device, and 1.8° C. difference in the wounds that received standard of care treatment (FIG. 70). The data presented suggests the delivery of the electroceutical therapy did not pose any adverse reactions on the skin and that the presence of the exciflex device did not present any adverse effects from a materials standpoint.

Taken together, the pre-clinical results provided herein suggest that the exciflex system delivered reliable electroceutical therapy, minimized unnecessary dressing changes and wound bed disruption, and improved healing. Advantageously, the development of a closed-loop electroceutical system that can conform to wounds of varied geometries over a one-week duration while being able to observe wound healing through an absorbent and flexible wound dressing without removal of the bandage enables broad translation of the present application for clinical applications.

### Example 3

This Example compares an electrode (e.g., CB-AFTIDerm) constructed according to one aspect of the present application with one of the compositions (i.e., containing PVA, glycerol and CB; referred to therein as the “PGB” sample) disclosed by Gu et al. (ACS Appl. Mater. Interfaces 2020, 12, 36, 40815-40827) (“Gu”). As discussed in detail below, the electrode of the present application exhibits unexpectedly superior properties—including hardness, Young's Modulus, conductivity and resistivity—when compared to the composition of Gu.

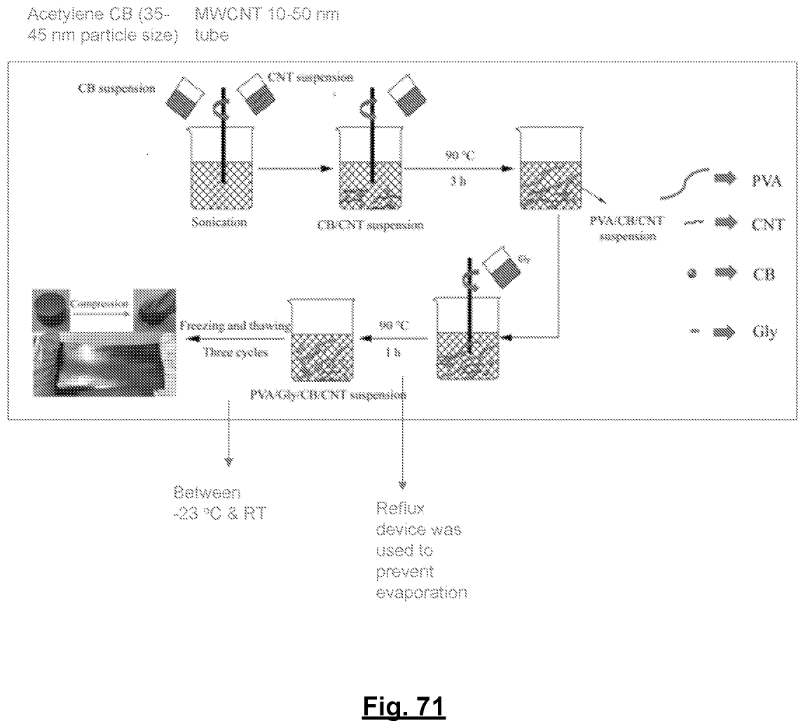

An electrode of the present application (CB-AFTIDerm) was fabricated as described in Example 1 and as shown in FIG. 11. The compositions of Gu were fabricated as described therein and as shown in FIG. 71. Briefly, PVA (1799, MW: ˜75000 g/mol, 99% hydrolysis) and Triton X-100 was purchased from Shanghai Jinrilai Co., Ltd. Glycerol was obtained from Kelong chemical Co. (Chengdu, China). The diameter and length of multi-walled carbon nanotube (MWCNTs) purchased from Conjutek Co. Taiwan was in the range of 10-50 nm and 100-200 μm, respectively. The particle size of acetylene black (CB) was 35-45 nm and it was purchased from Shanghai Jinrilai Co., Ltd. Distilled water was used throughout the experiment. PVA/Gly/CB/CNT organohydrogels were prepared by freezing and thawing cycle between −23° C. and room temperature. The organohydrogels with different content of CNT, CB and Gly were prepared using the following process as shown in FIG. 71. Table 51 of Gu lists the experimental ingredients and nomenclature of as-prepared hydrogel and organohydrogels (note: the weight of CB (0.03 g) in the PGB sample is erroneous; the correct weight of CB should have been reported as 0.3 g). Table S2 of Gu lists the mass percentage of each component for the different samples, including the PGB sample.

Electrodes (CB-AFTIDerm) of various formulations were prepared as shown below:

| | | | Glycerol | PVA |
|---|---|---|---|---|
| | | Water | (liquid) | (powder) |
| | CB weight | volume* | weight | weight |
| CB % | (g) | (mL) | (g) | (g) |
| 50 | 2 | 50 | 2.5 | 1.5 |
| 55 | 2.2 | 50 | 2.5 | 1.5 |
| 60 | 2.4 | 50 | 2.5 | 1.5 |
| 65** | 1.73 | 35 | 1.67 | 1 |
| 70* | 1.87 | 35 | 1.67 | 1 |

*water was used to enhance mixing CB with PVA/Gly. After 48 hours, water completely evaporated.
**at higher CB concentrations, reducing process output (water + PVA/Gly) was better for mixing.
*** Gly % = 2.5 g/50 mL = 5% of water.

Exemplary relevant calculations were performed as follows for 50% CB and 70% CB:

$$50 \% CB : Total PVA / Gly weight = 1.5 + 2.5 = 4 g$$

$$Total CB weight = 2 g$$

$$\frac{CB Weight ( g )}{PVA + Gly weight ( g )} = \frac{2}{4} = 0.5$$

$$\to ( CB : PVA / Gly ) = 1 : 2$$

$$50 \% CB = CB \vert PG 1 : 2$$

$$70 \% CB : Total PVA / Gly weight = 1.67 + 1 = 2.67 g$$

$$Total CB weight = 1.87 g$$

$$\frac{CB Weight ( g )}{PVA / Gly weight ( g )} = \frac{1.87}{2.67} = 0.7$$

$$\to ( CB : PVA / Gly ) = 7 : 10$$

The following tables illustrate the raw data and weight percentages (including water) used (respectively):

| | | | Glycerol | PVA | |
|---|---|---|---|---|---|
| | CB | Water | (liquid) | (powder) | Total |
| | weight | volume | weight | weight | weight |
| | (g) | (mL) | (g) | (g) | (g) |
| Electrode | 2 | 50 | 2.5 | 1.5 | 56 |
| (CB-AFTIDerm) | | | | | |
| Gu | 0.3 | 6 | 3 | 1.8 | 11.1 |

| | CB weight % | Water % | Gly % | PVA % | Total |
|---|---|---|---|---|---|
| Electrode | 3.57 | 89.28 | 4.46 | 2.67 | 100 |
| (50% CB- | | | | | |
| AFTIDerm) | | | | | |
| Gu | 2.7 | 55.5 | 27.03 | 16.21 | 100 |

The following table illustrates weight percentages (excluding water) of the electrode (CB-AFTIDerm) of the present application:

| | | Glycerol | PVA | |
|---|---|---|---|---|
| | | (liquid) | (powder) | Total |
| | CB weight | weight | weight | weight |
| | (g) | (g) | (g) | (g) |
| Electrode (CB- | 2 | 2.5 | 1.5 | 6 |
| AFTIDerm) | | | | |
| Weight % | 33.33 | 41.67 | 25 | 100 |

An exemplary calculation of CB wt % of the electrode (CB-AFTIDerm) is illustrated below:

$$CB wt = \frac{CB weight ( g )}{PVA + Gly weight ( g ) + CB weight} = \frac{2}{6} = 0.3333$$

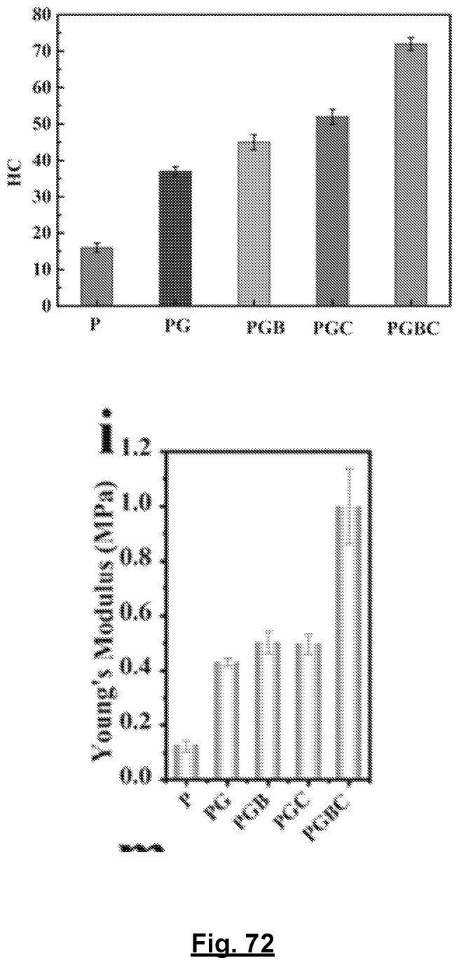

Hardness and Young's Modulus were compared for the electrode (CB-AFTIDerm) of the present application and the PGB sample composition of Gu. The results are illustrated in the table below as well as in FIG. 72.

| | Electrode (CB-AFTIDerm) | Gu |
|---|---|---|
| Hardness | 99.83* | 45 |
| Young's Modulus | 11.98 | 0.5 |

*Calculated from Qi equation (shown below), where E = 11.98 MPa

An estimate of the relation between ASTM D2240 type D hardness and the elastic modulus for a conical indenter with a 15° cone is given by Qi³:

$${S}_{p} = 100 - \frac{20 ( - 78.188 + \sqrt{6113.36 + 781.88 E )}}{E}$$

where S_{D} is the ASTM D2240 type D hardness, and E is in MPa.

Conductivity (S/m) and resistivity (Ω-m) were determined for the electrode (CB-AFTIDerm) of the present application, as shown in the table below:

| CB concentration (%) | Ave resistivity (Ω-m) | Ave conductivity (S/m) |
|---|---|---|
| 50 | 0.0748 | 31.37 |
| 55 | 0.0441 | 22.67 |
| 60 | 0.0304 | 32.90 |
| 65 | 0.00863 | 115.81 |
| 70 | 0.00604 | 165.57 |

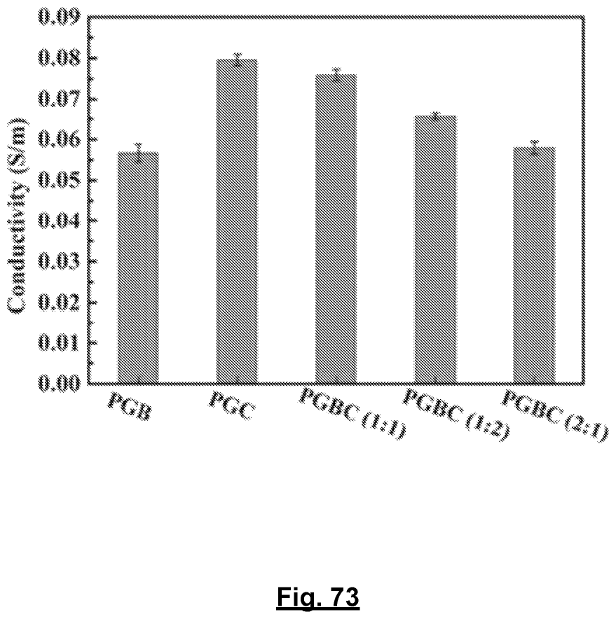

FIG. 73 shows conductivity of the Gu compositions. Surprisingly, when compared to the PGB sample of Gu, the electrode (50% CB-AFTIDerm) of the present application exhibited significantly better resistivity and conductivity (see table below).

| | Electrode (50% CB-AFTIDerm) | Gu |
|---|---|---|
| Conductivity (S/m) | 13.37 | 0.056 |
| Resistivity (Ω-m) | 0.0748 | 17.85 |

In summary, and as shown in the table below, the electrode (e.g., 50% CB-AFTIDerm) of the present application when compared to the PGB sample of Gu exhibited several surprising properties that make the electrodes of the present disclosure superior for bioelectronic applications, such as those disclosed herein.

| | Electrode | Gu |
|---|---|---|
| | (50% CB-AFTIDerm) | (PGB sample) |
| CB wt % | 3.57 | 2.7 |
| Gly wt % | 4.46 | 27.03 |
| PVA wt % | 2.67 | 16.21 |
| Water wt % | 89.28 | 55.05 |
| Hardness | 99.83 | 45 |
| Young's Modulus (MPa) | 11.98 | 0.5 |
| Resistivity (Ω-m) | 0.0748 | 17.85 |
| Conductivity (S/m) | 13.37 | 0.056 |

From the above description of the present disclosure, those skilled in the art will perceive improvements, changes and modifications. Such improvements, changes, and modifications are within the skill of those in the art and are intended to be covered by the appended claims. All patents, patent applications, and publications cited herein are incorporated by reference in their entirety.

1. A wound dressing for application against a wound site of a subject, the wound dressing comprising:
a transparent, moisture absorbing layer having a wound side and an opposed outer side; and
an adhesive layer that is connected to at least a portion of the wound side of the moisture absorbing layer, wherein the adhesive layer facilitates attachment of the wound dressing to a non-wounded perimeter of the wound site;
wherein the moisture absorbing layer absorbs moisture from the wound site, without swelling, to promote healing of the wound site.

2. The wound dressing of claim 1, wherein the moisture absorbing layer comprises a thermoplastic material and a polyol compound.

3. The wound dressing of claim 1, wherein the thermoplastic material is poly(vinyl alcohol) (PVA).

4. The wound dressing of claim 1, wherein the concentration of the PVA in the moisture absorbing layer is about 1 to 5 wt %.

5. (canceled)

6. The wound dressing of claim 1, wherein the polyol compound is glycerol.

7. The wound dressing of claim 1, wherein the concentration of the glycerol in the moisture absorbing layer is about 3 to 15 wt %.

8. (canceled)

9. (canceled)

10. The wound dressing of claim 1, wherein the moisture absorbing layer remains transparent after absorbing liquid from the wound site.

11. The wound dressing of claim 1, wherein the moisture absorbing layer absorbs liquid from the wound site, without swelling, for a period of about 1 hour to about 14 days following contact of the wound dressing with the wound site.

12. The wound dressing of claim 1, wherein the moisture absorbing layer includes one or more bioactive agents for delivery into tissue comprising the wound site or a surrounding non-wound site.

13. The wound dressing of claim 1, wherein the moisture absorbing layer is free of any exogenous conductive elements.

14. A device comprising:
a transparent, moisture absorbing layer having a wound side and an opposed outer side, wherein the moisture absorbing layer absorbs moisture, without swelling, from a wound site;
an occlusive layer positioned against at least a portion of the outer side of the moisture absorbing layer, wherein the occlusive layer comprises flexible circuitry that defines a plurality of electrical contacts; and
a plurality of temperature sensors coupled to the flexible circuitry;
wherein each temperature sensor of the plurality of temperature sensors is in electrical communication with a respective contact of the plurality of contacts of the flexible circuitry.

15. The device of claim 14, further comprising a control module in electrical communication with the plurality of temperature sensors, wherein the control module is operative to receive and/or store a signal from each temperature sensor of the plurality of temperature sensors.

16. The device of claim 14, wherein the plurality of temperature sensors comprises at least a first temperature sensor and a second temperature sensor, wherein the control module is further operative to:
receive a temperature measurement from between the first temperature sensor and the second temperature sensor; and
transmit, to a remote device, a signal corresponding to the temperature measurement.

17. The device of claim 14, further comprising an adhesive patch connected to the moisture absorbing layer and/or the occlusive layer.

18. The device of claim 14, further comprising a plurality of electrodes disposed over the wound side of the moisture absorbing layer, or disposed over a wound side of the occlusive layer, wherein each electrode of the plurality of electrodes is an elastomeric nanocomposite and is in electrical communication with a respective contact of the plurality of contacts of the flexible circuitry.

19. A method for monitoring healing of a wound site, the method comprising the steps of:
positioning the device of claim 14 on a subject having a wound site so that a first temperature sensor of the plurality of temperature sensors is positioned within or over the wound site and a second temperature sensor of the plurality of temperature sensors is positioned at a location spaced apart from the wound site; and
determining, by a processing device, a status of the wound based on a temperature difference between the first temperature sensor and the second temperature sensor.

20. (canceled)

21. (canceled)

22. A method for healing a wound site of a subject, the method comprising:
applying the wound dressing of claim 1, or the device of any one of claims 14-18, over the wound site;
optionally applying, by the electrodes, a series of electrical stimulations to the wound site; and
leaving the wound dressing or the device over the wound site for a period of time until the wound site is healed.

23. (canceled)

24. (canceled)

25. An electrode comprising: carbon black; a thermoplastic material; and a polyol compound; wherein the electrode is formulated to absorb moisture without swelling.

26. The electrode of claim 25, wherein the concentration of the carbon black in the electrode is between about 35 and 60 wt %.

27. (canceled)

28. The electrode of claim 25, wherein the thermoplastic material is PVA.

29. The electrode of claim 25, wherein the concentration of the PVA in the electrode is about 1 to 5 wt %.

30. (canceled)

31. The electrode of claim 25, wherein the polyol compound is glycerol.

32. The electrode of claim 25, wherein the concentration of the glycerol in the electrode is about 3 to 15 wt %.

33. (canceled)

34. (canceled)

35. The electrode of claim 25, wherein at least one portion of the electrode is transparent.

36. The electrode of claim 35, wherein the at least one portion of the electrode that is transparent remains transparent after absorbing moisture.

37. The electrode of claim 25, wherein the electrode absorbs moisture, without swelling, for a period of about 1 hour to about 14 days.

38. The electrode of claim 25, wherein the electrode has a resistance of less than about 1Ω and a resistivity of less than about 1 Ω-cm.

39. (canceled)

40. The electrode of claim 25, wherein the electrode is physically free from contact with a conductive gel.

41. (canceled)

42. A device comprising:
a moisture absorbing layer having a wound side and an opposed outer side;
a plurality of electrodes disposed over the wound side of the moisture absorbing layer, wherein each electrode of the plurality of electrodes is an electrode of claim 25;
an occlusive layer positioned against the outer side of the moisture absorbing layer, wherein the occlusive layer comprises flexible circuitry that defines a plurality of electrical contacts; and
a plurality of temperature sensors coupled to the flexible circuitry;
wherein each electrode of the plurality of electrodes is in electrical communication with a respective contact of the plurality of contacts of the flexible circuitry.

43. (canceled)

44. The device of claim 42, further comprising:
a control module in electrical communication with the plurality of electrodes and the plurality of temperature sensors, wherein the control module is operative to:
control an electrical current between at least two electrodes of the plurality of electrodes to provide a series of electrical stimulations to a wound site; and
receive a signal from each temperature sensor of the plurality of temperature sensors.

45. The device of claim 42, wherein the plurality of electrodes comprises at least a first electrode and a second electrode, wherein the control module is further operative to:
receive an impedance measurement from between the first electrode and the second electrode; and
transmit, to a remote device, a signal corresponding to the impedance measurement.

46. The device of claim 42, further comprising an adhesive patch connected to the moisture absorbing layer and/or the occlusive layer.

47. A method for healing a wound site of a subject, the method comprising:
applying the device of claim 42 over the wound site; and
applying, by the electrodes, a series of electrical stimulations to the wound site until the wound site is healed, the series of electrical stimulations being based on a received temperature measurement and/or a received impedance measurement from between the first and second electrodes and from between each temperature sensor of the plurality of temperature sensors, respectively.

48. (canceled)

49. (canceled)
